(19) United States
(12) Patent Application Publication
(10) Pub. No.: US 2024/0307564 A1
(43) Pub. Date: Sep. 19, 2024

(54) ANTI-CLDN18.2 ANTIBODY CONJUGATES

(71) Applicants: SUZHOU TRANSCENTA THERAPEUTICS CO., LTD., Suzhou, Jiangsu (CN); BEIJING CANCER HOSPITAL, Beijing (CN)

(72) Inventors: Xueming QIAN, Suzhou, Jiangsu (CN); Hua ZHU, Beijing (CN); Fei TENG, Suzhou, Jiangsu (CN); Hongjun LI, Suzhou, Jiangsu (CN); Zhi YANG, Beijing (CN); Yi GU, Suzhou, Jiangsu (CN); Jin DING, Beijing (CN); Feng WANG, Beijing (CN)

(21) Appl. No.: 18/546,980

(22) PCT Filed: Feb. 18, 2022
(86) PCT No.: PCT/CN2022/076810

§ 371 Date: Aug. 18, 2023

(30) Foreign Application Priority Data
Feb. 19, 2021 (WO) PCT/CN2021/076910
Jan. 28, 2022 (WO) PCT/CN2022/074750

(51) Int. Cl. A61K 51/10; C07K 16/28

(52) U.S. Cl. CPC A61K 51/1045; A61K 51/1096; C07K 16/28; A61K 2123/00; C07K 2317/24; C07K 2317/33; C07K 2317/732; C07K 2317/92

(57) ABSTRACT

The present disclosure provides herein conjugates of anti-CLDN18.2 antibodies or antigen-binding fragments thereof with radionuclides, the pharmaceutical composition containing the same and the uses thereof in imaging, patient screening, treatment process monitoring and efficacy evaluation.

### FIELD OF THE INVENTION

The present invention relates to nuclear medicine and, in particular, preparation and application of radio-labeled CLDN18.2 antibody conjugates.

### BACKGROUND

Worldwide, the incidence and mortality of gastric cancer ranked fifth and third respectively. The incidence rate of gastric cancer ranks second in China. At present, trastuzumab, a monoclonal antibody targeting human epidermal growth factor receptor 2 (HER2), is the only drug with survival benefit in the first-line treatment of advanced gastric cancer. In recent years, although good news of new drugs targeting HER2 (antibody-drug conjugates, bispecific antibodies, small molecule inhibitors, etc.) has been reported frequently, it is still unable to get rid of the embarrassing situation that the proportion of HER2 positive gastric cancer patients is low (only 10-12%), most of them develop drug resistance within one year, and lack of accurate and effective targeted drugs after drug resistance. Therefore, exploring new targets for gastric cancer treatment is the key to break through the bottleneck of advanced gastric cancer treatment.

The FAST study in 2016 validated Claudin18.2 (claudin 18 splice variant 2, CLDN18.2), the “new star” target of gastric cancer, through a series of experiments on IMAB362, a monoclonal antibody targeting CLDN18.2. However, to date, no reports of anti-CLDN18.2 nuclide probe have been published.

It is of great significance to develop molecular probes with high specificity for the diagnosis of CLDN18.2 by using the comprehensive, non-invasive, real-time and dynamic characteristics of modern molecular imaging technology. Therefore, there exists significant needs for novel anti-CLDN18.2 nuclide probe and therapeutics.

### SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one aspect, the present disclosure provides an anti-CLDN18.2 antibody conjugate, comprising anti-CLDN18.2 antibody or an antigen-binding fragment thereof conjugated to a radionuclide wherein the radionuclide comprises a therapeutic radionuclide or a diagnostic radionuclide.

In some embodiments, the therapeutic radionuclide is selected from the group consisting of ¹¹¹In, ^{111m}In, ¹⁷⁷Lu, ²¹²Bi, ²¹³Bi, ²¹¹At, ⁶²Cu, ⁶⁴Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, ¹⁹⁹Au, and ²¹¹Pb.

In some embodiments, the diagnostic radionuclide is selected from the group consisting of ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹ln, ¹²³l, ¹²⁴l, ¹²⁵l, ¹³¹l, ¹⁴²Pr, ¹⁴³Pr, ¹⁴⁹Pm, ¹⁵³Sm, ^{154″1581}Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴lr, ¹⁹⁸Au, ¹⁹⁹Au, ²¹¹At, ²¹¹Pb, ²¹²Bi, ²¹²Pb, ²¹³Bi, ²²³Ra and ²²⁵Ac.

In some embodiments, the diagnostic radionuclide is detectable by positron emission tomography (PET) or single-photon emission computerized tomography (SPECT).

In some embodiments, the radionuclide is selected from the group consisting of ⁶⁴Cu, ⁶⁷Cu, ⁸⁹Zr, ¹²⁴I, ⁸⁶Y, ⁹⁰Y, ¹¹¹In, ^{123/131}I, ¹⁷⁷Lu, ¹¹C, ¹⁴C, ⁴¹Ca, ⁶⁷Ga, ⁶⁸Ga, ¹³N, ¹⁵O, ⁴⁴Sc, ¹⁸F, ^{99m}Tc, and ^{90m}Tc.

In some embodiments, the radionuclide is ¹²⁴I or ¹²³I or ¹³¹I.

In some embodiments, the radionuclide is ⁶⁴Cu, ⁶⁷Cu or ⁸⁹Zr.

In some embodiments, the ⁶⁴Cu, ⁶⁷Cu or ⁸⁹Zr is labeled to the antibody or an antigen-binding fragment thereof via a chelator.

In some embodiments, the chelator comprises three or more atoms for chelation, wherein each atom is selected from the group consisting of nitrogen, sulfur, oxygen, and phosphorus.

In some embodiments, the chelator comprises DFO (derferoxamine), DOTA (1,4,7,10-tetraazacyclododecane-1,4,7,10-tetracetic acid), DTPA (NR-diethylenetriaminepentacetic acid), NOTA (1,4,7-triazacyclononane-1,4,7-acetic acid), 1,4,7,10-tetraazacyclotridecane-N,N′,N″,N′″-tetraacetic acid (herein abbreviated as TRITA); 1,4,8,11-tetraazacyclotetradecane-N,N′,N″,N′″-tetraacetic acid (herein abbreviated as TETA); and 1,5,9,13-tetraazacyclohexadecane-N,N′,N″,N′″-tetraacetic acid (abbreviated herein abbreviated as HETA), ethylenediaminetetraacetic acid (herein abbreviated as EDTA), or diethylenetriaminepentaacetic acid (DTPA).

In some embodiments, the radionuclide is ⁶⁴Cu or ⁶⁷Cu and the chelator comprises TETA, NOTA, NODA, or NODGA, or wherein the radionuclide is ⁸⁹Zr and the chelator comprises DFO.

In some embodiments, the anti-CLDN18.2 antibody or an antigen-binding fragment thereof comprises heavy chain HCDR1, HCDR2 and HCDR3 and/or light chain LCDR1, LCDR2 and LCDR3 sequences, wherein:
the HCDR1 sequence comprises GYNMN (SEQ ID NO: 1), or TYFIGVG (SEQ ID NO: 13), or a homologue sequence of at least 80% sequence identity thereof;
the HCDR2 sequence comprises X₁IDPYYX₂X₃TX₄YNQKFX₅G (SEQ ID NO: 32), or HIWWNDNKYYNTALKS (SEQ ID NO: 15), or a homologue sequence of at least 80% sequence identity thereof;
the HCDR3 sequence comprises X₆X₇X₈GNAFDY (SEQ ID NO: 33), or MGSGAWFTY (SEQ ID NO: 17), or a homologue sequence of at least 80% sequence identity thereof;
the LCDR1 sequence comprises KSSQX₉LX₁₀NX₁₁GNX₁₂KNYLT (SEQ ID NO: 34) or a homologue sequence of at least 80% sequence identity thereof; the LCDR2 sequence comprises WASTRX₁₃S (SEQ ID NO: 35) or a homologue sequence of at least 80% sequence identity thereof;
the LCDR3 sequence comprises QNDYX₁₄X₁₅PX₁₆T (SEQ ID NO: 36) or a homologue sequence of at least 80% sequence identity thereof;
wherein X₁ is N or Y or H, X₂ is G or V, X₃ is A or G or T, X₄ is R or T or S, X₅ is K or R, X₆ is S or M, X₇ is Y or F, X₈ is Y or H, X₉ is S or N, X₁₀ is L or F, X₁₁ is S or N, X₁₂ is Q or L, X₁₃ is E or K, X₁₄ is S or Y, X₁₅ is F or Y and X₁₆ is F or L.

In some embodiments, the anti-CLDN18.2 antibody or an antigen-binding fragment thereof comprises:
a heavy chain variable region comprises a HCDR1 comprising the sequence of SEQ ID NO: 1, a HCDR2 comprising the sequence of SEQ ID NO: 3, and a HCDR3 comprising the sequence of SEQ ID NO: 5; and a light chain variable region comprises a LCDR1 comprising the sequence of SEQ ID NO: 2, a LCDR2 comprising the sequence of SEQ ID NO: 4, and a LCDR3 comprising the sequence of SEQ ID NO: 6;
a heavy chain variable region comprises a HCDR1 comprising the sequence of SEQ ID NO: 1, a HCDR2 comprising the sequence of SEQ ID NO: 7, and a HCDR3 comprising the sequence of SEQ ID NO: 5; and a light chain variable region comprises a LCDR1 comprising the sequence of SEQ ID NO: 2, a LCDR2 comprising the sequence of SEQ ID NO: 4, and a LCDR3 comprising the sequence of SEQ ID NO: 8;
a heavy chain variable region comprises a HCDR1 comprising the sequence of SEQ ID NO: 1, a HCDR2 comprising the sequence of SEQ ID NO: 9, and a HCDR3 comprising the sequence of SEQ ID NO: 11; and a light chain variable region comprises a LCDR1 comprising the sequence of SEQ ID NO: 10, a LCDR2 comprising the sequence of SEQ ID NO: 4, and a LCDR3 comprising the sequence of SEQ ID NO: 6;
a heavy chain variable region comprises a HCDR1 comprising the sequence of SEQ ID NO: 13, a HCDR2 comprising the sequence of SEQ ID NO: 15, and a HCDR3 comprising the sequence of SEQ ID NO: 17; and a light chain variable region comprises a LCDR1 comprising the sequence of SEQ ID NO: 2, a LCDR2 comprising the sequence of SEQ ID NO: 4, and a LCDR3 comprising the sequence of SEQ ID NO: 12;
a heavy chain variable region comprises a HCDR1 comprising the sequence of SEQ ID NO: 1, a HCDR2 comprising the sequence of SEQ ID NO: 19, and a HCDR3 comprising the sequence of SEQ ID NO: 21; and a light chain variable region comprises a LCDR1 comprising the sequence of SEQ ID NO: 14, a LCDR2 comprising the sequence of SEQ ID NO: 16, and a LCDR3 comprising the sequence of SEQ ID NO: 18; or a heavy chain variable region comprises a HCDR1 comprising the sequence of SEQ ID NO: 1, a HCDR2 comprising the sequence of SEQ ID NO: 22, and a HCDR3 comprising the sequence of SEQ ID NO: 5; and a light chain variable region comprises a LCDR1 comprising the sequence of SEQ ID NO: 20, a LCDR2 comprising the sequence of SEQ ID NO: 4, and a LCDR3 comprising the sequence of SEQ ID NO: 6.

In some embodiments, the heavy chain variable region further comprises one or more of heavy chain HFR1, HFR2, HFR3 and HFR4, and/or the light chain variable region further comprises one or more of light chain LFR1, LFR2, LFR3 and LFR4, wherein:
the HFR1 comprises QVQLVQSGAEVKKPGASVKVSCKASGYX₁₇FT (SEQ ID NO: 54) or a homologous sequence of at least 80% sequence identity thereof,
the HFR2 comprises WVX₁₈QAPGQGLEWX₁₉G (SEQ ID NO: 55) or a homologous sequence of at least 80% sequence identity thereof, the HFR3 sequence comprises RVTX₂₀TIDKSTSTVYMELSSLRSEDTAVYYCAR (SEQ ID NO: 56) or a homologous sequence of at least 80% sequence identity thereof,
the HFR4 comprises WGQGTTVTVSS (SEQ ID NO: 57) or a homologous sequence of at least 80% sequence identity thereof,
the LFR1 comprises DIVMTQSPDSLAVSLGERATX₂₁NC (SEQ ID NO: 58) or a homologous sequence of at least 80% sequence identity thereof,
the LFR2 comprises WYQQKPGQPPKLLIY (SEQ ID NO: 59) or a homologous sequence of at least 80% sequence identity thereof,
the LFR3 comprises GVPDRFX₂₂GSGSGTDFTLTISSLQAEDVAVYYC (SEQ ID NO: 60) or a homologous sequence of at least 80% sequence identity thereof, and
the LFR4 comprises FGGGTKVEIK (SEQ ID NO: 61) or a homologous sequence of at least 80% sequence identity thereof,
   wherein X₁₇ is T or S, X is R or K, X₁₉ is M or I, X₂₀ is M or L, X₂₁ is I or M, and X₂₂ is S or T.

In some embodiments, the HFR1 comprises a sequence selected from the group consisting of SEQ ID NOs: 62 and 63,
the HFR2 comprises a sequence selected from the group consisting of SEQ ID NOs: 64 and 65,
the HFR3 comprises the sequence selected from the group consisting of SEQ ID NOs: 66 and 67,
the HFR4 comprises a sequence of SEQ ID NO: 57,
the LFR1 comprises the sequence from the group consisting of SEQ ID NOs: 68 and 69,
the LFR2 comprises a sequence of SEQ ID NO: 59,
the LFR3 comprises a sequence selected from the group consisting of SEQ ID NOs: 70 and 71, and
the LFR4 comprises a sequence of SEQ ID NO: 61.

In some embodiments, the heavy chain variable region comprises a sequence selected from the group consisting of SEQ ID NO: 25, SEQ ID NO: 27, SEQ ID NO: 29, SEQ ID NO: 37, SEQ ID NO: 39, SEQ ID NO: 41, SEQ ID NO: 43, SEQ ID NO: 45, and SEQ ID NO: 47, and a homologous sequence thereof having at least 80% sequence identity yet retaining specific binding affinity to CLDN18.2.

In some embodiments, the light chain variable region comprises a sequence selected from the group consisting of SEQ ID NO: 26, SEQ ID NO: 28, SEQ ID NO: 38, SEQ ID NO: 40, SEQ ID NO: 42, SEQ ID NO: 44, SEQ ID NO: 46, SEQ ID NO: 48, and a homologous sequence thereof having at least 80% sequence identity yet retaining specific binding affinity to CLDN18.2.

In some embodiments,
the heavy chain variable region comprising the sequence of SEQ ID NO: 23 and a light chain variable region comprising the sequence of SEQ ID NO: 24;
the heavy chain variable region comprises a sequence of SEQ ID NO: 25 and the light chain variable region comprises a sequence of SEQ ID NO: 26;
the heavy chain variable region comprises a sequence of SEQ ID NO: 27 and the light chain variable region comprises a sequence of SEQ ID NO: 28;
the heavy chain variable region comprises a sequence of SEQ ID NO: 29 and the light chain variable region comprises a sequence of SEQ ID NO: 26, or 28;
the heavy chain variable region comprises a sequence of SEQ ID NO: 37 and the light chain variable region comprises a sequence of SEQ ID NO: 38;
the heavy chain variable region comprises a sequence of SEQ ID NO: 39 and the light chain variable region comprises a sequence of SEQ ID NO: 40;
the heavy chain variable region comprises a sequence of SEQ ID NO: 41 and the light chain variable region comprises a sequence of SEQ ID NO: 42;
the heavy chain variable region comprises a sequence of SEQ ID NO: 43 and the light chain variable region comprises a sequence of SEQ ID NO: 44;
the heavy chain variable region comprises a sequence of SEQ ID NO: 45 and the light chain variable region comprises a sequence of SEQ ID NO: 46; or
the heavy chain variable region comprises a sequence of SEQ ID NO: 47 and the light chain variable region comprises a sequence of SEQ ID NO: 48.

In some embodiments, the anti-CLDN18.2 antibody or an antigen-binding fragment thereof further comprises one or more amino acid residue substitutions or modifications yet retains specific binding affinity to human CLDN18.2.

In some embodiments, at least one of the substitutions or modifications is in one or more of the CDR sequences, and/or in one or more of the non-CDR regions of the VH or VL sequences.

In some embodiments, the an anti-CLDN18.2 antibody or an antigen-binding fragment thereof further comprises an immunoglobulin constant region, optionally a constant region of human Ig, or optionally a constant region of human IgG.

In some embodiments, the constant region comprises a constant region of human IgG1, IgG2, IgG3, or IgG4.

In some embodiments, the constant region of human IgG1 comprises SEQ ID NO: 49, or a homologous sequence having at least 80% sequence identity thereof.

In some embodiments, the anti-CLDN18.2 antibody or an antigen-binding fragment thereof is humanized.

In some embodiments, the anti-CLDN18.2 antibody or an antigen-binding fragment thereof is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

In another aspect, the present disclosure provides a pharmaceutical composition comprising the antibody conjugate provided herein and one or more pharmaceutically acceptable carriers.

In some embodiments, radiochemical purity is at least 95% (e.g. 97%, 98%, 99%).

In another aspect, the present disclosure provides a method of obtaining an image of a site of interest in a subject, the method comprising the steps of.
a) administering to the subject an effective amount of the antibody conjugate provided herein and/or the pharmaceutical composition provided herein; and
b) subjecting the site of interest of the subject to positron emission tomography (PET) or SPECT;
c) identifying a detectable signal from the radionuclide in the subject;
d) generating an image of the detectable signal, thereby obtaining an image of the site of interest in the subject.

In some embodiments, the site of interest is a site expressing or suspected of expressing claudin 18.2.

In some embodiments, the site of interest has or is suspected of having tumor.

In another aspect, the present disclosure provides a method of detecting or visualizing claudin 18.2 expression in a subject in a non-invasive manner, comprising the steps of.
a) administering to the subject an effective amount of the antibody conjugate provided herein and/or the pharmaceutical composition provided herein; and
b) subjecting the subject to positron emission tomography (PET) or SPECT;
c) identifying a detectable signal from the radionuclide in a site of interest of the subject;
d) determining or visualizing claudin18.2 expression in the site of interest of the subject based on the identified detectable signal.

In some embodiments, the method further comprises administering a therapeutically effective amount of anti-claudin 18.2 therapy to the subject identified as having claudin18.2 expression in the site of interest.

In some embodiments, the method further comprises determining distribution of claudin18.2 expression in the site of interest of the subject based on the identified detectable signal.

In some embodiments, the method further comprises determining heterogeneity of claudin18.2 expression in the site of interest of the subject.

In some embodiments, the site of interest is tumor.

In another aspect, the present disclosure provides a method of monitoring therapeutic efficacy, responsiveness to treatment, or development of resistance or recurrence, or metastasis, in a subject in a non-invasive manner, wherein the subject has received treatment for a therapeutic period, comprising the steps of:
a) administering to the subject an effective amount of the antibody conjugate provided herein and/or the pharmaceutical composition provided herein; and
b) subjecting the subject to positron emission tomography (PET) or SPECT;
c) identifying a detectable signal from the radionuclide in a site of interest of the subject;
d) determining post-treatment claudin18.2 expression in a site of interest of the subject based on the identified detectable signal;
e) comparing the post-treatment claudin18.2 expression level or distribution, with a baseline claudin18.2 expression level or distribution, respectively, obtained from the subject before the therapeutic period, to determine post-treatment change in the claudin18.2 expression level or distribution in the subject; and
f) determining the therapeutic efficacy, responsiveness to treatment, or development of resistance or recurrence, or metastasis based on the change determined in step (e).

In some embodiments, the baseline claudin18.2 expression level or distribution is determined using a similar method.

In some embodiments, the baseline claudin18.2 expression level or distribution is determined before the therapeutic period (i.e. pre-treatment), by administering to the subject an effective amount of the antibody conjugate provided herein and/or the pharmaceutical composition provided herein; and subjecting the subject to positron emission tomography (PET) or SPECT, identifying a detectable signal from the radionuclide in a site of interest of the subject, and determining the baseline claudin18.2 expression in a site of interest of the subject based on the identified detectable signal. In some embodiments, the site of interest for determination of baseline claudin 18.2 expression is the same as, or at least comparable to, the site of interest for determination of post-treatment claudin 18.2 expression.

In some embodiments, an increased level or spread of claudin 18.2 expression indicates reduced therapeutic efficacy, reduced responsiveness to treatment, or presence of resistance or presence of recurrence.

In some embodiments, the subject has cancer.

In another aspect, the present disclosure provides a method of treating a CLDN18.2 related disease or condition in a subject in need thereof, comprising administering to the subject a therapeutically effective amount of the anti-CLDN18.2 antibody conjugate provided herein and/or the pharmaceutical composition provided herein.

In some embodiments, the disease or condition is cancer, optionally CLDN18.2-expressing cancer.

In some embodiments, the subject is identified as having CLDN18.2-expressing cancer.

In another aspect, the present disclosure provides a kit comprising the anti-CLDN18.2 antibody conjugate provided herein or the composition provided herein.

In another aspect, the present disclosure provides a kit comprising a first anti-CLDN18.2 antibody conjugate provided herein, and a second anti-CLDN18.2 antibody conjugate provided herein, wherein the first anti-CLDN18.2 antibody conjugate comprises a diagnostic radionuclide, and the second anti-CLDN18.2 antibody conjugate comprises a therapeutic radionuclide.

In some embodiments, the kit is for use in the method provided herein.

In another aspect, the present disclosure provides use of the anti-CLDN18.2 antibody conjugate provided herein or the composition provided herein in the manufacture of a medicament for use in the method of obtaining an image of a site of interest in a subject provided herein, or for use in the method of detecting or visualizing claudin 18.2 expression in a subject in a non-invasive manner provided herein, or for use in the method of monitoring therapeutic efficacy, responsiveness to treatment, or development of resistance or recurrence, or metastasis, in a subject in a non-invasive manner provided herein, wherein the anti-CLDN18.2 antibody conjugate comprises a diagnostic radionuclide.

In another aspect, the present disclosure provides use of the anti-CLDN18.2 antibody conjugate provided herein or the composition provided herein in the manufacture of a medicament for use in the method of treating a CLDN18.2 related disease or condition in a subject in need thereof provided herein, wherein the anti-CLDN18.2 antibody conjugate comprises a therapeutic radionuclide.

In another aspect, the present disclosure provides a method of preparing the anti-CLDN18.2 antibody conjugate provided herein, comprising reacting an anti-CLDN18.2 antibody or an antigen-binding fragment thereof with an iodide compound labeled with ¹²⁴I, ¹²³I or ¹³¹I, in the presence of an enzymatic or chemical oxidant.

In some embodiments, the chemical oxidant is N-bromosuccinimide, Iodogen, or Chloramine-T.

In another aspect, the present disclosure provides a method of preparing the anti-CLDN18.2 antibody conjugate provided herein, comprising conjugating an anti-CLDN18.2 antibody or an antigen-binding fragment thereof with a chelator to obtain a chelator-antibody conjugate, and reacting the chelator-antibody conjugate with ⁶⁴Cu, or ⁸⁹Zr.

In some embodiments, the chelator comprises DFO (derferoxamine), DOTA (1,4,7,10-tetraazacyclododecane-1,4,7,10-tetracetic acid), DTPA (NR-diethylenetriaminepentacetic acid), NOTA (1,4,7-triazacyclononane-1,4,7-acetic acid), TRITA (1,4,7,10-tetraazacyclotridecane-N,N′,N″,N′″-tetraacetic acid); TETA (1,4,8,11-tetraazacyclotetradecane-N,N′,N″,N′″-tetraacetic acid); and HETA (1,5,9,13-tetraazacyclohexadecane-N,N′,N″,N′″-tetraacetic acid), EDTA (ethylenediaminetetraacetic acid), NETA({4-[2-(bis-carboxymethylamino)-ethyl]-7-carboxymethyl-[1,4,7]triazonan-1-yl}-acetic acid), TACN-TM (N,N′,N″, tris(2-mercaptoethyl)1,4,7-triazacyclononane), TRAP (1,4,7-triazacyclononane-1,4,7-tris[methyl(2-carboxyethyl)phosphinic acid]), CP256,PCTA (3,6,9,15-tetraazabicyclo[9.3.1]pentadeca-1(15), 11,13-triene-3,6,9,-triacetic acid), porphyrins, polyamines, crown ethers, bis-thiosemicarbazones, polyoximes, and derivatives thereof.

### BRIEF DESCRIPTION OF FIGURES

FIG. 1 shows radiolabeling rate and radiochemical purity of ¹²⁴I-18B10 probe: A) Radiochemical purity after purification; B) Radiochemical purity after purification; C) Radiochemical purity after 1 h in PBS; D) Radiochemical purity after 48 h in PBS.

FIG. 2 shows the uptake of ¹²⁴I-18B10 and its control group in MKN45-CLND18.2 high/MKN45 cells.

FIG. 3 shows the biodistribution of ¹²⁴I-18B10 in normal Kunming mice.

FIG. 4 shows the Micro-PET imaging comparison of ¹²⁴I-18B10 and its control group and 18F-FDG in CLDN18.2 positive/negative PDX model in Example 5 of the invention.

FIG. 5A-B are bar graphs showing binding signal of chimeric antibodies to mutated hCLDN 18.2 variants using epitope mapping. Binding of 18B10-C was completely lost when E56 was mutated to Q. This change also applied to IMAB362 and other chimeric antibodies, except for 59A9-C. Other amino acids, such as A42, N45, also contributed to binding of IMAB362 and other antibodies at some extent but not so for 18B10-C.

FIG. 6 shows the biodistribution of ¹²⁴I-18B10 in PDX mice.

FIG. 7 shows the biodistribution of ¹²⁴I-18B10 in patients derived from the first five patients' images.

FIG. 8 shows the accumulations of ¹²⁴I-18B10 in patients' ovarian lesion.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (V_{H}) and a first, second, and third constant region (C_{H1}, C_{H2}, C_{H3}, respectively); mammalian light chains are classified as λ or κ, while each light chain consists of a variable region (V_{L}) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding domains disclosed herein may be defined or identified by the conventions of Kabat, IMGT, AbM, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); N. R. Whitelegg et al, Protein Engineering, v13(12), 819-824 (2000); Chothia, C. et al., Nature. December 21-28; 342(6252):877-83 (1989); Kabat E. A. et al., National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant regions of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain). In certain embodiments, the antibody provided herein encompasses any antigen-binding fragments thereof.

As used herein, the term “antigen-binding fragment” refers to an antibody fragment formed from a fragment of an antibody comprising one or more CDRs, or any other antibody portion that binds to an antigen but does not comprise an intact native antibody structure. Examples of antigen-binding fragment include, without limitation, a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody. An antigen-binding fragment is capable of binding to the same antigen to which the parent antibody binds. In certain embodiments, an antigen-binding fragment may comprise one or more CDRs from a particular human antibody.

“Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

“Fab′” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

“F(ab′)₂” refers to a dimer of Fab′ that comprises two light chains and part of two heavy chains.

“Fc” with regard to an antibody refers to that portion of the antibody consisting of the second and third constant regions of a first heavy chain bound to the second and third constant regions of a second heavy chain via disulfide bond. IgG and IgM Fc regions contain three heavy chain constant regions (second, third and fourth heavy chain constant regions in each chain). It can be obtained by papain digestion of an antibody. The Fc portion of the antibody is responsible for various effector functions such as ADCC, ADCP and CDC, but does not function in antigen binding.

“Fv” with regard to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)). A “scFv dimer” refers to a single chain comprising two heavy chain variable regions and two light chain variable regions with a linker. In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or eptipoes) or different antigens (or eptipoes). In other embodiments, a “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

“Single-chain Fv-Fc antibody” or “scFv-Fc” refers to an engineered antibody consisting of a scFv connected to the Fc region of an antibody.

“Camelized single domain antibody,” “heavy chain antibody,” “nanobody” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 June 15 (2007)). “Diabodies” include small antibody fragments with two antigen-binding sites, wherein the fragments comprise a VH domain connected to a V_{L} domain in a single polypeptide chain (V_{H}-V_{L} or V_{L}—VH) (see, e.g., Holliger P. et al., Proc Natl Acad Sci USA. July 15; 90(14):6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same of different antigens (or epitopes).

A “domain antibody” refers to an antibody fragment containing only the variable region of a heavy chain or the variable region of a light chain. In certain embodiments, two or more V_{H} domains are covalently joined with a peptide linker to form a bivalent or multivalent domain antibody. The two V_{H} domains of a bivalent domain antibody may target the same or different antigens.

In certain embodiments, a “(dsFv)₂” comprises three peptide chains: two V_{H} moieties linked by a peptide linker and bound by disulfide bridges to two V_{L} moieties.

In certain embodiments, a “bispecific ds diabody” comprises V_{H1}-V_{L2} (linked by a peptide linker) bound to V_{L1}-V_{H2} (also linked by a peptide linker) via a disulfide bridge between V_{H1} and V_{L1}.

In certain embodiments, a “bispecific dsFv” or “dsFv-dsFv′” comprises three peptide chains: a V_{H1}-V_{H2} moiety wherein the heavy chains are bound by a peptide linker (e.g., a long flexible linker) and paired via disulfide bridges to V_{L1} and V_{L2} moieties, respectively. Each disulfide paired heavy and light chain has a different antigen specificity.

The term “humanized” as used herein means that the antibody or antigen-binding fragment comprises CDRs derived from non-human animals, FR regions derived from human, and when applicable, constant regions derived from human. In certain embodiments, the amino acid residues of the variable region framework of the humanized CLDN18.2 antibody are substituted for sequence optimization. In certain embodiments, the variable region framework sequences of the humanized CLDN18.2 antibody chain are at least 65%, 70%, 75%, 80%, 85%, 90%, 95% or 100% identical to the corresponding human variable region framework sequences.

The term “chimeric” as used herein refers to an antibody or antigen-binding fragment that has a portion of heavy and/or light chain derived from one species, and the rest of the heavy and/or light chain derived from a different species. In an illustrative example, a chimeric antibody may comprise a constant region derived from human and a variable region derived from a non-human species, such as from mouse.

The term “germline sequence” refers to the nucleic acid sequence encoding a variable region amino acid sequence or subsequence that shares the highest determined amino acid sequence identity with a reference variable region amino acid sequence or subsequence in comparison to all other known variable region amino acid sequences encoded by germline immunoglobulin variable region sequences. The germline sequence can also refer to the variable region amino acid sequence or subsequence with the highest amino acid sequence identity with a reference variable region amino acid sequence or subsequence in comparison to all other evaluated variable region amino acid sequences. The germline sequence can be framework regions only, complementarity determining regions only, framework and complementarity determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. Germline sequences can be determined, for example, through the publicly available international ImMunoGeneTics database (IMGT) and V-base.

“Anti-CLDN18.2 antibody” or “an antibody against CLDN18.2” as used herein refers to an antibody that is capable of specific binding to CLDN18.2 (e.g. human or non-human CLDN18.2) with a sufficient affinity, for example, to provide for diagnostic and/or therapeutic use.

The term “affinity” as used herein refers to the strength of non-covalent interaction between an immunoglobulin molecule (i.e. antibody) or fragment thereof and an antigen.

The term “specific binding” or “specifically binds” as used herein refers to a non-random binding reaction between two molecules, such as for example between an antibody and an antigen. In certain embodiments, the antibodies or antigen-binding fragments provided herein specifically bind to human and/or non-human CLDN18.2 with a binding affinity (K_{D}) of ≤10⁻⁶ M (e.g., ≤5×10⁻⁷ M, ≤2×10⁻⁷ M, ≤10⁻⁷ M, ≤5×10⁻⁸ M, ≤2×10⁻⁸ M, ≤, 10⁻⁹ M, ≤5×10⁻⁹ M, ≤4×10⁻⁹M, ≤3×10⁻⁹M, ≤2×10⁻⁹ M, or ≤10⁻⁹ M. K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry method. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least 10 to 100 times over the background.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum correspondence. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm. In certain embodiments, the non-identical residue positions may differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference.

As used herein, a “homologue sequence” and “homologous sequence” are used interchangeable and refer to polynucleotide sequences (or its complementary strand) or amino acid sequences that have sequences identity of at least 80% (e.g. at least 85%, 88%, 90%, 91%, 92%, 93%, 94%, 95%, 96%, 97%, 98%, 99%) to another sequences when optionally aligned.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An isolated “nucleic acid” or “polynucleotide” are used interchangeably and refer to the sequence of an isolated nucleic acid molecule. In certain embodiments, an “isolated antibody or antigen-binding fragment thereof” refers to the antibody or antigen-binding fragments having a purity of at least 60%, 70%, 75%, 80%, 81%, 82%, 83%, 84%, 85%, 86%, 87%, 88%, 89%0, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The ability to “block binding” or “compete for the same epitope” as used herein refers to the ability of an antibody or antigen-binding fragment to inhibit the binding interaction between two molecules (e.g. human CLDN18.2 and an anti-CLDN18.2 antibody) to any detectable degree. In certain embodiments, an antibody or antigen-binding fragment that blocks binding between two molecules inhibits the binding interaction between the two molecules by at least 50%. In certain embodiments, this inhibition may be greater than 60%, greater than 70%, greater than 80%, or greater than 90%.

The term “antibody conjugate” as used herein refers to the linkage of an antibody or an antigen binding fragment thereof with another agent, such as a radionuclide.

The term “subject” includes human and non-human animals. Non-human animals include all vertebrates, e.g., mammals and non-mammals, such as non-human primates, mouse, rat, cat, rabbit, sheep, dog, cow, chickens, amphibians, and reptiles. Except when noted, the terms “patient” or “subject” are used herein interchangeably.

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “CLDN18.2” refers to Claudin-18 splice variant 2 derived from mammals, such as primates (e.g. humans, monkeys) and rodents (e.g. mice). In certain embodiments, CLDN18.2 is human CLDN18.2. Exemplary sequence of human CLDN18.2 includes human CLDN18.2 protein (NCBI Ref Seq No. NP_001002026.1, or SEQ ID NO: 30). Exemplary sequence of CLDN18.2 includes Mus musculus (mouse) CLDN18.2 protein (NCBI Ref Seq No. NP_001181852.1), Macaca fascicularis (crab-eating macaque) CLDN18.2 protein (NCBI Ref Seq No. XP_015300615.1). CLDN18.2 is expressed in a cancer cell. In one embodiment said CLDN18.2 is expressed on the surface of a cancer cell.

The term “CLDN18.1” refers to Claudin-18 splice variant 1 derived from mammals, such as primates (e.g. humans, monkeys) and rodents (e.g. mice). In certain embodiments, CLDN18.1 is human CLDN18.1. Exemplary sequence of human CLDN18.1 includes human CLDN18.1 protein (NCBI Ref Seq No. NP_057453.1, or SEQ ID NO: 31), Mus musculus (mouse) CLDN18.2 protein (NCBI Ref Seq No. NP_001181851.1), Macaca fascicularis (crab-eating macaque) CLDN18.2 protein (NCBI Ref Seq No. XP_005545920.1).

A “CLDN18.2-related” disease or condition as used herein refers to any disease or condition caused by, exacerbated by, or otherwise linked to increased or decreased expression or activities of CLDN18.2. In some embodiments, the CLDN18.2 related condition is, for example, cancer.

“Cancer” as used herein refers to any medical condition characterized by malignant cell growth or neoplasm, abnormal proliferation, infiltration or metastasis, and includes both solid tumors and non-solid cancers (e.g. hematologic malignancies) such as leukemia. As used herein “solid tumor” refers to a solid mass of neoplastic and/or malignant cells.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

Anti-CLDN18.2 antibody Conjugates

The present disclosure provides anti-CLDN18.2 antibody radionuclide conjugates.

Claudin-18 (CLDN18) molecule (Genbank accession number: splice variant 1 (CLDN18A1 or CLDN18.1): NP_057453, NM_016369, and splice variant 2 (CLDN18A2 or CLDN18.2): NM_001002026, NP_001002026) is an integral transmembrane protein with a molecular weight of approximately 27.9/27.72 kD. CLDN18 proteins are located within the tight junctions of epithelia and endothelia that organize a network of interconnected strands of intramembranous particles between adjacent cells. CLDN18 and occludin are the most prominent transmembrane protein components in the tight junctions. Due to their strong intercellular adhesion properties, these tight junction proteins create a primary barrier to prevent and control the paracellular transport of solutes, and also restrict the lateral diffusion of membrane lipids and proteins to maintain cellular polarity.

CLDN18 displays several different conformations, which may be selectively addressed by antibodies (see Sahin U, Koslowski M, Dhaene K, et al. Claudin-18 splice variant 2 is a pan-cancer target suitable for therapeutic antibody development[J]. Clinical Cancer Research, 2008, 14(23): 7624-7634). CLDN18-Conformation-1 has all four hydrophobic regions serving as the transmembrane domains (TM), and two extracellular loops (loop1 embraced by hydrophobic region 1 and hydrophobic region 2; loop2 embraced by hydrophobic region 3 and 4) are formed, as described for the vast majority of CLDN family members. A second conformation (CLDN18-Conformation-2) implies that, as described for PMP22, the second and third hydrophobic domains do not fully cross the plasma membrane so that portion (loop D3) between the first and fourth transmembrane domains is extracellular. A third conformation (CLDN18-Conformation-3) shows a large extracellular domain with two internal hydrophobic regions embraced by the first and fourth hydrophobic regions. Because of a classical N-glycosylation site in the loop D3, the CLDN-18 topology variants CLDN18 topology-2 and CLDN18 topology-3 harbor an additional extracellular N-glycosylation site.

CLDN18 has two different splice variants, which are present in both mouse and human. The splice variants CLDN18.1 and CLDN18.2 differ in the first 21 amino acids at the N-terminus that comprises the first TM and the loop1, whereas the protein sequences in the C-terminus are identical (see Niimi T, Nagashima K, Ward J M, et al. Claudin-18, a novel downstream target gene for the T/EBP/NKX2. 1 homeodomain transcription factor, encodes lung- and stomach-specific isoforms through alternative splicing[J]. Molecular and cellular biology, 2001, 21(21): 7380-7390.).

CLDN18.1 is selectively expressed on normal lung and stomach epithelia, whereas CLDN18.2 is only expressed on gastric cells. Most importantly, CLDN18.2 expression is restricted to the differentiated short-lived cells of stomach epithelium, but devoid from the gastric stem cell region. Using sensitive RT-PCR, both variants are not detectable in any other normal human organ. However, they are highly expressed in several cancer types including stomach, esophageal, pancreatic and lung tumors as well as human cancer cell lines (see Matsuda Y, Semba S, Ueda J, et al. Gastric and intestinal claudin expression at the invasive front of gastric carcinoma[J]. Cancer science, 2007, 98(7): 1014-1019.).

Without wishing to be bound to any theories, it is believed that the molecular and functional characteristics of CLDN18.2 make it a highly interesting target for antibody-based cancer diagnosis and therapy. These include (i) absence of CLDN18 from the majority of toxicity relevant normal tissues, (ii) restriction of CLDN18.2 variant expression to a dispensable cell population as differentiated gastric cells that can be replenished by target-negative stem cells of the stomach, (iii) potential differential glycosylation between normal and neoplastic cells, and (iv) the presence of different conformational topologies.

It has been found that the molecular weight of the CLDN18 protein differs between tumors and adjacent normal tissues. The higher molecular weight CLDN18 protein is observed in healthy tissues, which can be decreased to the same molecular weight as observed in tumor by treatment of the normal tissue lysates with deglycosylating compound PNGase F. This suggests that CLDN18 is less N-glycosylated in tumor as compared to its normal tissue counterpart. A classical N-glycosylation motif is in amino acid residue 116 within the loop D3 domain of the CLDN18 molecule. The molecular weight difference and the inferred structural difference may represent an altered epitope for antibody binding.

In addition, CLDN18 as a tight junction protein may also contribute to a good specificity for diagnosis, and a good therapeutic window for treatment. Since tumor cells express CLDNs but often do not form the classical tight junctions by homotypic and heterotypic association of CLDNs as found in normal epithelial tissue, they likely have a considerable pool of free CLDNs that are amenable to extracellular antibody binding and immunotherapy. It is possible that binding epitopes of CLDNs in healthy epithelium are shielded within the tight junctions from being accessed to antibody binding.

The present disclosure provides anti-CLDN18.2 antibody conjugates, comprising anti-CLDN18.2 antibody or an antigen-binding fragment thereof conjugated to a radionuclide.

i. Radionuclides

Radionuclide are agents characterized by an unstable nucleus that is capable of undergoing radioactive decay. Radionuclides useful within the present disclosure include gamma-emitters, positron-emitters, Auger electron-emitters, X-ray emitters and fluorescence-emitters. Radionuclides may be either therapeutic or diagnostic.

In certain embodiments, the radionuclides are therapeutic. Radionuclides decaying by short-range high LET emissions such as betas, alphas and Auger electrons are highly cytotoxic, and therefore useful for labeling substances designed for radiotherapeutic purposes. Therapeutic radionuclide can have a decay energy in the range of 20 to 6,000 keV, for example in the ranges 60 to 200 keV for an Auger emitter, 100-2,500 keV for a beta emitter, and 4,000-6,000 keV for an alpha emitter. A person skilled in the art can select a suitable therapeutic radionuclide to be used in the antibody conjugates provided herein, based on factors including, the radionuclide half-life, the energy of the emitted particles, the maximum range that the emitted particle can travel, sufficient concentration and prolonged retention of the radionuclide by the cancer cell, among others.

Examples of therapeutic radionuclides include, but are not limited to ¹¹¹In, ^{111m}In, ¹⁷⁷Lu, ²¹²Bi, ²¹³Bi, ²¹¹At, ⁶²Cu, ⁶⁴Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, ¹⁹⁹Au, and ²¹¹Pb.

Examples of additional therapeutic radionuclides that substantially decay with Auger-emitting particles include, Co-58, Ga-67, Br-80m, Tc-99m, Rh-103m, Pt-109, In-111, Sb-119, I-125, Ho-161, Os-189m and Ir-192.

Examples of further additional radionuclides that substantially decay with generation of alpha-particles include, but are not limited to: Dy-152, At-211, Bi-212, Ra-223, Rn-219, Po-215, Bi-211, Ac-225, Fr-221, At-217, Bi-213 and Fm-255.

Additional potential therapeutic radioisotopes include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ^{125m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb and the like.

In certain embodiments, the radionuclides are diagnostic.

Diagnostic radionuclide can be used as an imaging agent. An imaging agent can indicate position of radionuclide and adherents thereto, in a cell or tissue of an animal or human subject, or a cell or tissue under in vitro conditions. In certain embodiments, the radionuclides are those which can be detected externally in a non-invasive manner following administration in vivo. Radionuclides for diagnostic use such as imaging agents are preferably with relatively low cytotoxicity but decay with emissions suitable for imaging. Radionuclides having diagnostic uses can include, for example, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹ln, ¹²³l, ¹²⁴l, ¹²⁵l, ¹³¹l, ¹⁴²Pr, ¹⁴³Pr, ¹⁴⁹Pm, ¹⁵³Sm, ^{154″1581}Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as diagnostic agents in accordance with the embodiments of the disclosure include, but are not limited to, ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

In certain embodiments, the radionuclides are radioactive metal ions, gamma-emitting radioactive halogens and positron-emitting radioactive non-metals. In certain embodiments, the radionuclide is detectable by positron emission tomography (PET) or single-photon emission computerized tomography (SPECT). For example, radionuclides that decay with gamma emissions are suitable for planar and Single Photon Computerized Emission Tomography (SPECT) imaging, while radionuclides that decay with positron (beta) emission and annihilation photons are suitable for Positron Emission Tomography (PET). Diagnostic Radionuclides label detectable by such as PET or SPECT imaging technology include, for example, without limitation, ⁶⁴Cu, ⁶⁷Cu, ⁸⁹Zr, ¹²⁴I, ⁸⁶Y, ⁹⁰Y, ¹¹¹In, ^{123/131}I, ¹⁷⁷Lu, ¹¹C, ¹⁴C, ⁴¹Ca ⁶⁷Ga, ⁶⁸Ga, ¹³N, ¹⁵O, ⁴⁴Sc, ¹⁸F, ^{99m}Tc, ^{90m}Tc and the like.

The term “Positron Emission Tomography (PET)” as used herein refers to a nuclear imaging technique used in the medical field to visualize a target tissue or organ or activities. PET measures the two annihilation photons that are produced back-to-back after positron emission from a radionuclide conjugated tracer molecule, which is chosen to mark a specific function in the body on a biochemistry level. PET provides molecular imaging of biological function instead of anatomy. PET allows examination of the patient by producing pictures of many functions of the human body unobtainable by other imaging techniques. After a short-lived positron-emitting radioactive tracer is injected into the subject, it can distribute within the body according to the physiologic pathways associated with the stable counterparts. When the tracer is a targeting molecule specifically directed to a target of interest, the tracer allows visualization of tissues or organs expressing such a target.

The term “SPECT” as used herein refers to “Single-Photon Emission Computed Tomography, which is a nuclear medicine tomographic imaging technique using gamma rays. It is similar to conventional nuclear medicine planar imaging using a gamma camera and able to provide true 3D information. This information is typically presented as cross-sectional slices through the patient, but can be freely reformatted or manipulated as required. Injection of a gamma-emitting radionuclide or its conjugate into the subject is needed for the imaging.

Methods of making radionuclide conjugated antibodies and antigen-binding fragments are known in the art. In general, methods are different for metallic radionuclides, and non-metallic radionuclides. Conjugation of metallic radionuclide typically require a chelator for the conjugation, which may not be necessary for non-metallic radionuclides.

Methods of conjugation of a metallic radionuclide to an antibody is known in the art, for example, via a suitable chelator (see, e.g., WO94/11026; Current Protocols in Immunology, Volumes 1 and 2, Coligen et al, Ed. Wiley-Interscience, New York, N.Y., Pubs. (1991)).

The term “chelator” as used herein refers to a chemical structure capable of binding a metal with two or more bonds. A chelator can have one or more chelating groups that can bind to a metal ion. For example, a chelator can comprises at least one heteroatom suitable for coordination to a metal ion, and sequester a metal ion from aqueous solution.

In some embodiments, the chelator comprises three or more atoms for chelation, wherein each atom is selected from the group consisting of nitrogen, sulfur, oxygen, and phosphorus.

Examples of chelators that may be used according to the disclosure include, but are not limited to, DFO (derferoxamine), DOTA (1,4,7,10-tetraazacyclododecane-1,4,7,10-tetracetic acid), DTPA (NR-diethylenetriaminepentacetic acid), NOTA (1,4,7-triazacyclononane-1,4,7-acetic acid), TRITA (1,4,7,10-tetraazacyclotridecane-N,N′,N″,N′″-tetraacetic acid); TETA (1,4,8,11-tetraazacyclotetradecane-N,N′,N″,N′″-tetraacetic acid); and HETA (1,5,9,13-tetraazacyclohexadecane-N,N′,N″,N′″-tetraacetic acid), EDTA (ethylenediaminetetraacetic acid), NETA({4-[2-(bis-carboxymethylamino)-ethyl]-7-carboxymethyl-[1,4,7]triazonan-1-yl}-acetic acid), TACN-TM (N,N′,N″, tris(2-mercaptoethyl)1,4,7-triazacyclononane), TRAP (1,4,7-triazacyclononane-1,4,7-tris[methyl(2-carboxyethyl)phosphinic acid]), CP256,PCTA (3,6,9,15-tetraazabicyclo[9.3.1]pentadeca-1(15), 11,13-triene-3,6,9,-triacetic acid), porphyrins, polyamines, crown ethers, bis-thiosemicarbazones, polyoximes, and derivatives thereof.

In some embodiments, the antibody conjugates provided herein comprises a metallic radionuclide conjugated to the anti-CLDN18.2 antibody or antigen-binding fragment thereof provided herein via a chelator. A person skilled in the art can selected a suitable chelator for a radionuclide based on knowledge known in the art, for example, as described by Price E. W et al, Chem. Soc. Rev., 2014, 43, 260-290.

In some embodiments, the metallic radionuclide can be ⁶⁴Cu, ⁶⁷Cu, ⁸⁹Zr, ⁸⁶Y, ⁹⁰Y, ¹¹¹In, ¹⁷⁷Lu, ⁶⁷Ga, ⁴⁴Sc, or ⁹⁹ⁿTc. In some embodiments, the radionuclide is ⁶⁴Cu, ⁶⁷Cu, or ⁸⁹Zr. In some embodiments, the ⁶⁴Cu, ⁶⁷Cu or ⁸⁹Zr is labeled to the antibody or an antigen-binding fragment thereof via a chelator.

In some embodiments, the chelator further comprises a reactive functional group for conjugation to the antibody. Chelates may be directly linked to antibodies or peptides, for example as disclosed in U.S. Pat. No. 4,824,659, incorporated herein in its entirety by reference. Such a reactive group allows the chelator to react with a functional group in one or more amino acid residues of the antibody, for example, free cysteine, lysine, and the like. Examples of reactive group include Maleimide, aminobenzyl, N-hydroxysuccinimide ester, and so on.

In certain embodiments, the chelator is conjugated to the antibody or antigen-binding fragment thereof provided herein by a bifunctional linker reagent. Examples of such bifunctional linkers include, without limitation, N-succinimidyl-3-(2-pyridyldithio) propionate (SPDP), succinimidyl-4-(N-maleimidomethyl) cyclohexane-1-carboxylate (SMCC), N-succinimidyl-4-(2-pyridylthio)pentanoate (SPP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), bis-active fluorine compounds (such as 1,5-difluom-2,4-dinitrobenzene), BMPS, EMCS, GMBS, HBVS, LC-SMCC, MBS, MPRH, SBAP, SIA, SIAB, SMPB, SMPH, sulfo-EMCS, sulfo-GMBS, sulfo-KMUS, sulfo-MBS, sulfo-SIAB, sulfo-SMCC, and sulfo-SMPB, and SVSG (succinimidyl-(4-vinylsulfone)benzoate). Those linker reagents are commercially available (e.g., from Pierce Biotechnology, Inc., Rockford, Ill., USA, see pages 467-498, 2003-2004 Applications Handbook and Catalog).

In some embodiments, the radionuclide is ⁶⁴Cu or ⁶⁷Cu, and the chelator comprises TETA, NOTA, NODA, or NODGA. In some embodiments, the radionuclide is ⁸⁹Zr and the chelator comprises DFO.

In some embodiments, the radionuclide is non-metallic. In some embodiments, the radionuclide comprises a radioactive halogen. In some embodiments, the radionuclide is ¹²⁴I or ¹²³I or ¹³¹I. Methods of preparing radioiodinated antibodies are known in the art, see, e.g., Grassi, J. et al. (1987). Radioiodination and Other Labeling Techniques. Handbook of Experimental Pharmacology, 91-141, Eclelman, W. C. et al, Cancer Research, 40, 3036-3042, 1998, which are incorporated herein by reference in its entirety.

In some embodiments, the ¹²⁴I or ¹²³I or ¹³¹I is labeled to at least one phenyl hydroxyl group of the antibody or an antigen-binding fragment thereof.

In certain embodiments, the antibody or an antigen-binding fragment thereof is directly iodinated by electrophilic substitution into tyrosine residues, in the presence of a radioiodine such as Na¹²⁴I. In certain embodiments, the antibody or an antigen-binding fragment thereof is indirectly iodinated by covalent linkage of a pre-labelled compound or a compound capable of post linkage labelling, for example, Bolton and Hunter reagent for peptides.

In some embodiments, the ¹²⁴I or ¹²³I or ¹³¹I is labeled to the antibody or an antigen-binding fragment thereof in the presence of an oxidant. Any suitable oxidants can be used, including chemical oxidant and enzymatic oxidant. Exemplary oxidant includes, without limitation, N-Bromosuccinimide, chloramine T, chlorine gas, or lactoperoxidase.

In some embodiments, the ¹²⁴I or ¹²³I or ¹³¹I is labeled to the antibody or an antigen-binding fragment thereof by N-bromosuccinimide (NBS) reaction.

In some embodiments, the antibody conjugates provided herein can be further conjugated to a nanoparticle. For example, nanoparticles can be used in therapeutic applications as drug carriers that, when conjugated to a CLDN18.2-specific antibody or fragment of the present invention, deliver chemotherapeutic agents, radiotherapeutic agents, toxins, or any other cytotoxic or anti-cancer agent known in the art to cancerous cells that overexpress CLDN18.2 on the cell surface.

In some embodiments, the antibody conjugates provided herein can be further conjugated to a drug (e.g., at the epsilon amino group of a lysine residue), and the carrier may incorporate an additional therapeutic or diagnostic agent.

i. Antibody Sequences

In certain embodiments, the antibody conjugates provided herein comprises an anti-CLDN18.2 antibody or an antigen-binding fragment thereof comprising heavy chain HCDR1, HCDR2 and HCDR3 and/or light chain LCDR1, LCDR2 and LCDR3 sequences, wherein
the HCDR1 sequence comprises GYNMN (SEQ ID NO: 1), or TYFIGVG (SEQ ID NO: 13), or a homologue sequence of at least 80% sequence identity thereof;
the HCDR2 sequence comprises X₁IDPYYX₂X₃TX₄YNQKFX₅G (SEQ ID NO: 32), or HIWWNDNKYYNTALKS (SEQ ID NO: 15), or a homologue sequence of at least 80% (or at least 85%, 90%, 95%) sequence identity thereof;
the HCDR3 sequence comprises X₆X₇X₈GNAFDY (SEQ ID NO: 33), or MGSGAWFTY (SEQ ID NO: 17), or a homologue sequence of at least 80% sequence identity thereof;
the LCDR1 sequence comprises KSSQX₉LX₁₀NX₁₁GNX₁₂KNYLT (SEQ ID NO: 34) or a homologue sequence of at least 80% (or at least 85%, 90%, 95%) sequence identity thereof;
the LCDR2 sequence comprises WASTRX₁₃S (SEQ ID NO: 35) or a homologue sequence of at least 80% sequence identity thereof; and
the LCDR3 sequence comprises QNDYX₁₄X₁₅PX₁₆T (SEQ ID NO: 36) or a homologue sequence of at least 80% sequence identity thereof;
wherein X₁ is N or Y or H, X₂ is G or V, X₃ is A or G or T, X₄ is R or T or S, X₅ is K or R, X₆ is S or M, X₇ is Y or F, X₈ is Y or H, X₉ is S or N, X₁₀ is L or F, X₁₁ is S or N, Xu is Q or L, X₁₃ is E or K, X₁₄ is S or Y, X₁₅ is F or Y and X₁₆ is F or L.

In certain embodiments, the antibody conjugates provided herein comprises an anti-CLDN18.2 antibody or an antigen-binding fragment thereof, wherein the heavy chain variable region comprises:
a) a HCDR1 comprises a sequence selected from SEQ ID NO: 1, and SEQ ID NO: 13,
b) a HCDR2 comprises a sequence selected from SEQ ID NO: 3, SEQ ID NO: 7, SEQ ID NO: 9, SEQ ID NO: 15, SEQ ID NO: 19, and SEQ ID NO: 22, and
c) a HCDR3 comprises a sequence selected from SEQ ID NO: 5, SEQ ID NO: 11, SEQ ID NO: 17, and SEQ ID NO: 21, and/or a light chain variable region comprising:
d) a LCDR1 comprises a sequence of SEQ ID NO: 2, SEQ ID NO: 10, SEQ ID NO: 14, and SEQ ID NO: 20,
e) a LCDR2 comprises a sequence of SEQ ID NO: 4, and SEQ ID NO: 16, and
f) a LCDR3 comprises a sequence selected from SEQ ID NO: 6, SEQ ID NO: 8, SEQ ID NO: 12, and SEQ ID NO: 18.

In certain embodiments, the heavy chain variable region is selected from the group consisting of:
a) a heavy chain variable region comprising a HCDR1 comprising the sequence of SEQ ID NO: 1, a HCDR2 comprising the sequence of SEQ ID NO: 3, and a HCDR3 comprising the sequence of SEQ ID NO: 5;
b) a heavy chain variable region comprising a HCDR1 comprising the sequence of SEQ ID NO: 1, a HCDR2 comprising the sequence of SEQ ID NO: 7, and a HCDR3 comprising the sequence of SEQ ID NO: 5;
c) a heavy chain variable region comprising a HCDR1 comprising the sequence of SEQ ID NO: 1, a HCDR2 comprising the sequence of SEQ ID NO: 9, and a HCDR3 comprising the sequence of SEQ ID NO: 11;
d) a heavy chain variable region comprising a HCDR1 comprising the sequence of SEQ ID NO: 13, a HCDR2 comprising the sequence of SEQ ID NO: 15, and a HCDR3 comprising the sequence of SEQ ID NO: 17;
e) a heavy chain variable region comprising a HCDR1 comprising the sequence of SEQ ID NO: 1, a HCDR2 comprising the sequence of SEQ ID NO: 19, and a HCDR3 comprising the sequence of SEQ ID NO: 21; and
f) a heavy chain variable region comprising a HCDR1 comprising the sequence of SEQ ID NO: 1, a HCDR2 comprising the sequence of SEQ ID NO: 22, and a HCDR3 comprising the sequence of SEQ ID NO: 5.

In certain embodiments, the light chain variable region is selected from the group consisting of:
a) a light chain variable region comprising a LCDR1 comprising the sequence of SEQ ID NO: 2, a LCDR2 comprising the sequence of SEQ ID NO: 4, and a LCDR3 comprising the sequence of SEQ ID NO: 6;
b) a light chain variable region comprising a LCDR1 comprising the sequence of SEQ ID NO: 2, a LCDR2 comprising the sequence of SEQ ID NO: 4, and a LCDR3 comprising the sequence of SEQ ID NO: 8;
c) a light chain variable region comprising a LCDR1 comprising the sequence of SEQ ID NO: 10, a LCDR2 comprising the sequence of SEQ ID NO: 4, and a LCDR3 comprising the sequence of SEQ ID NO: 6;
d) a light chain variable region comprising a LCDR1 comprising the sequence of SEQ ID NO: 2, a LCDR2 comprising the sequence of SEQ ID NO: 4, and a LCDR3 comprising the sequence of SEQ ID NO: 12;
e) a light chain variable region comprising a LCDR1 comprising the sequence of SEQ ID NO: 14, a LCDR2 comprising the sequence of SEQ ID NO: 16, and a LCDR3 comprising the sequence of SEQ ID NO: 18; and
f) a light chain variable region comprising a LCDR1 comprising the sequence of SEQ ID NO: 20, a LCDR2 comprising the sequence of SEQ ID NO: 4, and a LCDR3 comprising the sequence of SEQ ID NO: 6.

In certain embodiments, in the antibody conjugates provided herein:
a) the heavy chain variable region comprises a HCDR1 comprising the sequence of SEQ ID NO: 1, a HCDR2 comprising the sequence of SEQ ID NO: 3, and a HCDR3 comprising the sequence of SEQ ID NO: 5; and the light chain variable region comprises a LCDR1 comprising the sequence of SEQ ID NO: 2, a LCDR2 comprising the sequence of SEQ ID NO: 4, and a LCDR3 comprising the sequence of SEQ ID NO: 6;
b) the heavy chain variable region comprises a HCDR1 comprising the sequence of SEQ ID NO: 1, a HCDR2 comprising the sequence of SEQ ID NO: 7, and a HCDR3 comprising the sequence of SEQ ID NO: 5; and the light chain variable region comprises a LCDR1 comprising the sequence of SEQ ID NO: 2, a LCDR2 comprising the sequence of SEQ ID NO: 4, and a LCDR3 comprising the sequence of SEQ ID NO: 8;
c) the heavy chain variable region comprises a HCDR1 comprising the sequence of SEQ ID NO: 1, a HCDR2 comprising the sequence of SEQ ID NO: 9, and a HCDR3 comprising the sequence of SEQ ID NO: 11; and the light chain variable region comprises a LCDR1 comprising the sequence of SEQ ID NO: 10, a LCDR2 comprising the sequence of SEQ ID NO: 4, and a LCDR3 comprising the sequence of SEQ ID NO: 6;
d) the heavy chain variable region comprises a HCDR1 comprising the sequence of SEQ ID NO: 13, a HCDR2 comprising the sequence of SEQ ID NO: 15, and a HCDR3 comprising the sequence of SEQ ID NO: 17; and the light chain variable region comprises a LCDR1 comprising the sequence of SEQ ID NO: 2, a LCDR2 comprising the sequence of SEQ ID NO: 4, and a LCDR3 comprising the sequence of SEQ ID NO: 12;
e) the heavy chain variable region comprises a HCDR1 comprising the sequence of SEQ ID NO: 1, a HCDR2 comprising the sequence of SEQ ID NO: 19, and a HCDR3 comprising the sequence of SEQ ID NO: 21; and the light chain variable region comprises a LCDR1 comprising the sequence of SEQ ID NO: 14, a LCDR2 comprising the sequence of SEQ ID NO: 16, and a LCDR3 comprising the sequence of SEQ ID NO: 18; or
f) the heavy chain variable region comprises a HCDR1 comprising the sequence of SEQ ID NO: 1, a HCDR2 comprising the sequence of SEQ ID NO: 22, and a HCDR3 comprising the sequence of SEQ ID NO: 5; and the light chain variable region comprises a LCDR1 comprising the sequence of SEQ ID NO: 20, a LCDR2 comprising the sequence of SEQ ID NO: 4, and a LCDR3 comprising the sequence of SEQ ID NO: 6.

In certain embodiments, the antibody conjugates provided herein comprise one or more (e.g. 1, 2, 3, 4, 5, or 6) CDR sequences of a CLDN18.2 antibodies 7C12, 11F12, 26G6, 59A9, 18B10 and 12E9.

“7C12” as used herein refers to a mouse antibody having a heavy chain variable region of SEQ ID NO: 37, and a light chain variable region of SEQ ID NO: 38.

“11F12” as used herein refers to a mouse antibody having a heavy chain variable region of SEQ ID NO: 39, and a light chain variable region of SEQ ID NO: 40.

“26G6” as used herein refers to a mouse antibody having a heavy chain variable region of SEQ ID NO: 41, and a light chain variable region of SEQ ID NO: 42.

“59A9” as used herein refers to a mouse antibody having a heavy chain variable region of SEQ ID NO: 43, and a light chain variable region of SEQ ID NO: 44.

“18B10” as used herein refers to a mouse antibody having a heavy chain variable region of SEQ ID NO: 45, and a light chain variable region of SEQ ID NO: 46.

“12E9” as used herein refers to a mouse antibody having a heavy chain variable region of SEQ ID NO: 47, and a light chain variable region of SEQ ID NO: 48.

Table 1 shows the CDR sequences of these CLDN18.2 antibodies. The heavy chain and light chain variable region sequences are also provided below in Table 2.

TABLE 1
Sequences of CLDN18.2 antibodies' CDR region

| Antibody | Region | CDR1 | CDR2 | CDR3 |
|---|---|---|---|---|
| 7C12 | HCDR | SEQ ID NO: 1 | SEQ ID NO: 3 | SEQ ID NO: 5 |
| | | GYNMN | NIDPYYGATRYNQK | SYYGNAFD |
| | | | FKG | Y |
| | LCDR | SEQ ID NO: 2 | SEQ ID NO: 4 | SEQ ID NO: 6 |
| | | KSSQSLLNSGN | WASTRES | QNDYSFPFT |
| | | QKNYLT | | |
| 11F12 | HCDR | SEQ ID NO: 1 | SEQ ID NO: 7 | SEQ ID NO: 5 |
| | | GYNMN | YIDPYYGGTRYNQ | SYYGNAFD |
| | | | KFKG | Y |
| | LCDR | SEQ ID NO: 2 | SEQ ID NO: 4 | SEQ ID NO: 8 |
| | | KSSQSLLNSGN | WASTRES | QNDYSYPFT |
| | | QKNYLT | | |
| 26G6 | HCDR | SEQ ID NO: 1 | SEQ ID NO: 9 | SEQ ID NO: 11 |
| | | GYNMN | HIDPYYVTTTYNQK | SFYGNAFDY |
| | | | FRG | |
| | LCDR | SEQ ID NO: 10 | SEQ ID NO: 4 | SEQ ID NO: 6 |
| | | KSSQSLFNSGN | WASTRES | QNDYSFPFT |
| | | QKNYLT | | |
| 59A9 | HCDR | SEQ ID NO: 13 | SEQ ID NO: 15 | SEQ ID NO: 17 |
| | | TYFIGVG | HIWWNDNKYYNTA | MGSGAWFT |
| | | | LKS | Y |
| | LCDR | SEQ ID NO: 2 | SEQ ID NO: 4 | SEQ ID NO: 12 |
| | | KSSQSLLNSGN | WASTRES | QNDYYYPLT |
| | | QKNYLT | | |
| 18B10 | HCDR | SEQ ID NO: 1 | SEQ ID NO: 19 | SEQ ID NO: 21 |
| | | GYNMN | NIDPYYGGTSYNQK | MYHGNAFD |
| | | | FKG | Y |
| | LCDR | SEQ ID NO: 14 | SEQ ID NO: 16 | SEQ ID NO: 18 |
| | | KSSQSLLNSGN | WASTRKS | QNDYSYPLT |
| | | LKNYLT | | |
| 12E9 | HCDR | SEQ ID NO: 1 | SEQ ID NO: 22 | SEQ ID NO: 5 |
| | | GYNMN | NIDPYYGGTRYNQ | SYYGNAFD |
| | | | KFKG | Y |
| | LCDR | SEQ ID NO: 20 | SEQ ID NO: 4 | SEQ ID NO: 6 |
| | | KSSQNLLNNGN | WASTRES | QNDYSFPFT |
| | | QKNYLT | | |

TABLE 2
Sequences of mouse/chimeric antibody VH/VL

| | VH | VL |
|---|---|---|
| 7C12 | SEQ ID NO: 37 | SEQ ID NO: 38 |
| | EFQLQQSGPELEKPGASVRISCKT | DIVMTQSPSSLTVTAGEKVTMSC |
| | SGYSFTGYNMNWVKQSNGESLE | KSSQSLLNSGNQKNYLTWYQQKP |
| | WIGNIDPYYGATRYNQKFKGKAT | GQPPKLLIYWASTRESGVPDRFT |
| | LTVDKSSSTAYMQLKSLTSEDSAV | GSGSGTDFTLTISSVQAEDLAVYY |
| | YYCARSYYGNAFDYWGQGTTLT | CQNDYSFPFTFGSGTKLEIK |
| | VSS | |
| 11F12 | SEQ ID NO: 39 | SEQ ID NO: 40 |
| | EFQLQQSGPELEKPGASVRISCKT | DIVMTQSPSSLTVTAGEKVTMSC |
| | SGYSFTGYNMNWVKQSNGESLE | KSSQSLLNSGNQKNYLTWYQQKP |
| | WIGYIDPYYGGTRYNQKFKGKAT | GQPPKLLIYWASTRESGVPDRFT |
| | LTVDKSSSTAYMQLKSLTSEDSAV | GSGSGTDFTLTISSVQAEDLAVYY |
| | YYCARSYYGNAFDYWGQGTTLT | CQNDYSYPFTFGSGTKLEIK |
| | VSS | |
| 26G6 | SEQ ID NO: 41 | SEQ ID NO: 42 |
| | EFQLQQSGPELEKPGASVKISCKT | DIVMTQSPSSLTVTAGEKVTMSC |
| | SGYSFTGYNMNWVKQSNGQSLE | KSSQSLFNSGNQKNYLTWYQQKP |
| | WIGHIDPYYVTTTYNQKFRGKAT | GQPPKLLIYWASTRESGVPDRFT |
| | LTVDKSSSTAYMQLKSLTSEDSAV | GSGSGTDFTLTISSVQAEDLAVY |
| | YYCARSFYGNAFDYWGQGTTLT | YCQNDYSFPFTFGSGTKLEIK |
| | VSS | |
| 59A9 | SEQ ID NO: 43 | SEQ ID NO: 44 |
| | QITQKESGPGILQPSQTLSLTCSLS | DIVMTQSPSSLTVTAGEKVTMSC |
| | GFSLSTYFIGVGWIRQPSGKGLEW | KSSQSLLNSGNQKNYLTWYQQKP |
| | LAHIWWNDNKYYNTALKSRLTIS | GQPPKLLIYWASTRESGVPDRFT |
| | KDTSNNQVFLKIASVDTADTATYY | GSGSGTDFTLTISSVQAEDLAVY |
| | CARMGSGAWFTYWGQGTLVTVS | YCQNDYYYPLTFGSGTKLEIK |
| | A | |
| 18B10 | SEQ ID NO: 45 | SEQ ID NO: 46 |
| | EFQLQQSGPELEKPGASVRISCKT | DIVMTQSPSSLTVTAGEKVTMSC |
| | SGYSFTGYNMNWVKQSNGESLE | KSSQSLLNSGNLKNYLTWYQQK |
| | WIGNIDPYYGGTSYNQKFKGKAT | PGQPPKLLIYWASTRKSGVPDRF |
| | LTVDKSSSTAYMQLKSLTSEDSAV | TGSGSGTDFTLTLSSVQAEDLAV |
| | YYCARMYHGNAFDYWGQGTTLT | YYCQNDYSYPLTFGAGTKLELK |
| | VSS | |
| 12E9 | SEQ ID NO: 47 | SEQ ID NO: 48 |
| | EFQLQQSGPELEKPGASVRISCKT | DIVMTQSPSSLTVTAGEKVTMSC |
| | SGYSFTGYNMNWVKQSNGESLE | KSSQNLLNNGNQKNYLTWYQQ |
| | WIGNIDPYYGGTRYNQKFKGKAT | KPGQPPKLLIYWASTRESGVPDR |
| | LTVDKSSSTAYMQLKSLTSEDSAV | FTGSGSGTDFILTISSVQAEDLAV |
| | YYCARSYYGNAFDYWGQGTTLT | YYCQNDYSFPFTFGAGTKLELK |
| | VSS | |

The anti-CLDN18.2 antibodies or antigen-binding fragments thereof provided herein can be a monoclonal antibody, humanized antibody, chimeric antibody, recombinant antibody, bispecific antibody, labeled antibody, bivalent antibody, or anti-idiotypic antibody. A recombinant antibody is an antibody prepared in vitro using recombinant methods rather than in animals.

CDRs are known to be responsible for antigen binding, however, it has been found that not all of the 6 CDRs are necessarily indispensable or unchangeable. In other words, it is possible to replace or change or modify 1, 2, or 3 CDRs in anti-CLDN18.2 antibodies 7C12, 11F12, 26G6, 59A9, 18B10, or 12E9 (corresponding to any one of SEQ ID NOs: 1-22), yet substantially retain the specific binding affinity to CLDN18.2.

In certain embodiments, the anti-CLDN18.2 antibody conjugates provided herein comprise a heavy chain CDR3 sequence of one of the anti-CLDN18.2 antibodies 7C12, 11F12, 26G6, 59A9, 18B10, or 12E9. In certain embodiments, the antibody conjugates provided herein comprise a heavy chain CDR3 sequence of SEQ ID NOs: 5, 11, 17, and 21. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In some embodiments, the anti-CLDN18.2 antibody conjugates provided herein comprise all or a portion of the heavy chain variable domain and/or all or a portion of the light chain variable domain. In one embodiment, the anti-CLDN18.2 antibodies and the antigen-binding fragments provided herein is a single domain antibody which consists of all or a portion of the heavy chain variable domain provided herein. More information of such a single domain antibody is available in the art (see, e.g., U.S. Pat. No. 6,248,516).

In certain embodiments, the anti-CLDN18.2 antibody conjugates provided herein comprise suitable framework region (FR) sequences, as long as the antibodies and antigen-binding fragments thereof can specifically bind to CLDN18.2. The CDR sequences provided in Table 1 are obtained from mouse antibodies, but they can be grafted to any suitable FR sequences of any suitable species such as mouse, human, rat, rabbit, among others, using suitable methods known in the art such as recombinant techniques.

In certain embodiments, the anti-CLDN18.2 antibody conjugates provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g., rodent) antibody variable domain sequence is screened or BLASTed against a database of known human variable domain germline sequences, and the human sequence closest to the non-human query sequence is identified and used as the human scaffold for grafting the non-human CDR sequences (see, for example, Sims et al, (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623).

In certain embodiments, the humanized antibody conjugates provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived different human immunoglobulin genes, for example, FR sequences derived from one human antibody and constant region from another human antibody. In some embodiments, the humanized antibody or antigen-binding fragment comprise human heavy/light chain FR1-4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure to reduce or avoid immunogenicity and/or improve or retain the binding activity or binding affinity.

In certain embodiments, the humanized antibody conjugates provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FRs of a heavy or a light chain variable domain. In some embodiments, such change in amino acid residue could be present in heavy chain FR regions only, in light chain FR regions only, or in both chains. In certain embodiments, the one or more amino acid residues are mutated, for example, back-mutated to the corresponding residue found in the non-human parent antibody (e.g. in the mouse framework region) from which the CDR sequences are derived. Suitable positions for mutations can be selected by a skilled person following principles known in the art. For example, a position for mutation can be selected where: 1) the residue in the framework of the human germline sequence is rare (e.g. in less than 20% or less than 10% in human variable region sequence); 2) the position is immediately adjacent to one or more of the 3 CDR's in the primary sequence of the human germline chain, as it is likely to interact with residues in the CDRs; or 3) the position is close to CDRs in a 3-dimensional model, and therefore can have a good probability of interacting with amino acids in the CDR. The residue at the selected position can be mutated back to the corresponding residue in the parent antibody, or to a residue which is neither the corresponding residue in human germline sequence nor in parent antibody, but to a residue typical of human sequences, i.e. that occurs more frequently at that position in the known human sequences belonging to the same subgroup as the human germline sequence (see U.S. Pat. No. 5,693,762).

In certain embodiments, the humanized light and heavy chains of the present disclosure are substantially non-immunogenic in humans and retain substantially the same affinity as or even higher affinity than the parent antibody to CLDN18.2.

In certain embodiments, the humanized antibody conjugates thereof provided herein comprise one or more light chain FR sequences of human germline framework sequence VK/4-1, and/or one or more heavy chain FR sequences of human germline framework sequence VH/1-46, without or without back mutations. Back mutations can be introducted in to the human germline framework sequence, if needed. In certain embodiments, the humanized antibody 18B10 may contain one or more back mutations selected from the group consisting of: R71I, T73K, T28S, M69L, R38K, and M48I, all based on Kabat numbering, in heavy chain framework sequence VH/1-46. The humanized antibody 18B10 may contain one or more back mutations selected from the group consisting of: S63T, and I21M, all based on Kabat numbering, in light chain framework sequence VK/4-1.

In certain embodiments, in anti-CLDN18.2 antibody conjugates provided herein, the anti-CLDN18.2 antibody or an antigen-binding fragment thereof comprises a heavy chain variable region comprising the sequence selected from the group consisting of SEQ ID NO: 25, SEQ ID NO: 27, SEQ ID NO: 29, SEQ ID NO: 37, SEQ ID NO: 39, SEQ ID NO: 41, SEQ ID NO: 43, SEQ ID NO: 45, and SEQ ID NO: 47, and a homologous sequence thereof having at least 80% (e.g. at least 85%, 90%, 95%, 96%, 97%, 98%, or 99%) sequence identity yet retaining specific binding affinity to CLDN18.2, in particular human CLDN18.2.

In certain embodiments, in anti-CLDN18.2 antibody conjugates provided herein, the anti-CLDN18.2 antibody or an antigen-binding fragment thereof comprises a light chain variable region comprising the sequence selected from the group consisting of SEQ ID NO: 26, SEQ ID NO: 28, SEQ ID NO: 38, SEQ ID NO: 40, SEQ ID NO: 42, SEQ ID NO: 44, SEQ ID NO: 46, SEQ ID NO: 48, and a homologous sequence thereof having at least 80% (e.g. at least 85%, 90%, 95%, 96%, 97%, 98%, or 99%) sequence identity yet retaining specific binding affinity to CLDN18.2, in particular human CLDN18.2.

In certain embodiments, the anti-CLDN18.2 antibody conjugates provided herein comprises:
a heavy chain variable region comprising the sequence of SEQ ID NO: 25 and a light chain variable region comprising the sequence of SEQ ID NO: 26;
a heavy chain variable region comprising the sequence of SEQ ID NO: 27 and a light chain variable region comprising the sequence of SEQ ID NO: 28;
a heavy chain variable region comprising the sequence of SEQ ID NO: 29 and a light chain variable region comprising the sequence of SEQ ID NO: 26, or 28;
a heavy chain variable region comprising the sequence of SEQ ID NO: 37 and a light chain variable region comprising the sequence of SEQ ID NO: 38;
a heavy chain variable region comprising the sequence of SEQ ID NO: 39 and a light chain variable region comprising the sequence of SEQ ID NO: 40;
a heavy chain variable region comprising the sequence of SEQ ID NO: 41 and a light chain variable region comprising the sequence of SEQ ID NO: 42;
a heavy chain variable region comprising the sequence of SEQ ID NO: 43 and a light chain variable region comprising the sequence of SEQ ID NO: 44;
a heavy chain variable region comprising the sequence of SEQ ID NO: 45 and a light chain variable region comprising the sequence of SEQ ID NO: 46; or a heavy chain variable region comprising the sequence of SEQ ID NO: 47 and a light chain variable region comprising the sequence of SEQ ID NO: 48.

In certain embodiments, the anti-CLDN18.2 antibody conjugates provided herein further comprises one or more of heavy chain HFR1, HFR2, HFR3 and HFR4, and/or one or more of light chain LFR1, LFR2, LFR3 and LFR4, wherein:
the HFR1 comprises QVQLVQSGAEVKKPGASVKVSCKASGYX₁₇FT (SEQ ID NO: 54) or a homologous sequence of at least 80% (or at least 85%, 90%, 95%) sequence identity thereof,
the HFR2 comprises WVX₁₈QAPGQGLEWX₁₉G (SEQ ID NO: 55) or a homologous sequence of at least 80% (or at least 90%) sequence identity thereof,
the HFR3 sequence comprises RVTX₂₀TIDKSTSTVYMELSSLRSEDTAVYYCAR (SEQ ID NO: 56) or a homologous sequence of at least 80% (or at least 85%, 90%, 95%) sequence identity thereof,
the HFR4 comprises WGQGTTVTVSS (SEQ ID NO: 57) or a homologous sequence of at least 80% sequence identity thereof,
the LFR1 comprises DIVMTQSPDSLAVSLGERATX₂₁NC (SEQ ID NO: 58) or a homologous sequence of at least 80% (or at least 85%, 90%, 95%) sequence identity thereof,
the LFR2 comprises WYQQKPGQPPKLLIY (SEQ ID NO: 59) or a homologous sequence of at least 80% (or at least 85%, 90%) sequence identity thereof,
the LFR3 comprises GVPDRFX₂₂GSGSGTDFTLTISSLQAEDVAVYYC (SEQ ID NO: 60) or a homologous sequence of at least 80% (or at least 85%, 90%, 95%) sequence identity thereof, and
the LFR4 comprises FGGGTKVEIK (SEQ ID NO: 61) or a homologous sequence of at least 80% (or at least 90%) sequence identity thereof,
   wherein X₁₇ is T or S, X is R or K, X₁₉ is M or I, X₂₀ is M or L, X₂₁ is I or M, and X₂₂ is S or T.

In certain embodiments, the HFR1 comprises a sequence selected from the group consisting of SEQ ID NOs: 62 and 63, the HFR2 comprises a sequence selected from the group consisting of SEQ ID NOs: 64 and 65, the HFR3 comprises the sequence selected from the group consisting of SEQ ID NOs: 66 and 67, the HFR4 comprises a sequence of SEQ ID NOs: 57, the LFR1 comprises the sequence from the group consisting of SEQ ID NOs: 68 and 69, the LFR2 comprises a sequence of SEQ TD NO: 59, the LFR3 comprises a sequence selected from the group consisting of SEQ TD NOs: 70 and 71, and the LFR4 comprises a sequence of SEQ TD NO: 61.

TABLE 3-1
Framework (FR) sequences of humanized CLDN18.2
antibodies 18B10

| Antibody | | | | |
|---|---|---|---|---|
| chain | FR1 | FR2 | FR3 | FR4 |
| Hu18B10-Ha | SEQ ID NO: | SEQ ID NO: | SEQ ID NO: | SEQ ID NO: |
| | 62 | 64 | 66 | 57 |
| | QVQLVQSG | WVRQAPGQ | RVTMTIDKS | WGQGTTV |
| | AEVKKPGA | GLEWMG | TSTVYMELS | TVSS |
| | SVKVSCKA | | SLRSEDTAV | |
| | SGYTFT | | YYCAR | |
| Hu18B10-Hb | SEQ ID NO: | SEQ ID NO: | SEQ ID NO: | SEQ ID NO: |
| | 63 | 64 | 67 | 57 |
| | QVQLVQSG | WVRQAPGQ | RVTLTIDKST | WGQGTTV |
| | AEVKKPGA | GLEWMG | STVYMELSS | TVSS |
| | SVKVSCKA | | LRSEDTAVY | |
| | SGYSFT | | YCAR | |
| | SEQ ID | SEQ ID NO: | SEQ ID NO: | SEQ ID NO: |
| | NO:63 | 65 | 67 | 57 |
| Hu18B10-Hc | QVQLVQSG | WVKQAPGQ | RVTLTIDKST | WGQGTTV |
| | AEVKKPGA | GLEWIG | STVYMELSS | TVSS |
| | SVKVSCKA | | LRSEDTAVY | |
| | SGYSFT | | YCAR | |
| Hu18B10_La | SEQ ID NO: | SEQ ID NO: | SEQ ID NO: | SEQ ID NO: |
| | 68 | 59 | 70 | 61 |
| | DIVMTQSP | WYQQKPGQ | GVPDRFSGS | FGGGTKVE |
| | DSLAVSLG | PPKLLIY | GSGTDFTLTI | IK |
| | ERATINC | | SSLQAEDVA | |
| | | | VYYC | |
| Hu18B10 Lb | SEQ ID NO: | SEQ ID NO: | SEQ ID NO: | SEQ ID NO: |
| | 69 | 59 | 71 | 61 |
| | DIVMTQSP | WYQQKPGQ | GVPDRFSGS | FGGGTKVE |
| | DSLAVSLG | PPKLLIY | GSGTDFTLTI | IK |
| | ERATMNC | | SSLQAEDVA | |
| | | | VYYC | |

Table 3-2 illustrates sequences of the variable regions of humanized 18B10 antibodies.

TABLE 3-2
Sequences of humanized 18B10

| Antibody chain | Sequences |
|---|---|
| 18B10 HC germline | QVQLVQSGAEVKKPGASVKVSCKASGYTFTSYYMHWVR |
| | QAPGQGLEWMGIINPSGGSTSYAQKFQGRVTMTRDTST |
| | STVYMELSSLRSEDTAVYYCAR (SEQ ID NO: 23) |
| Hu18B10-Ha | QVQLVQSGAEVKKPGASVKVSCKASGYTFTGYNMNWVR |
| | QAPGQGLEWMGNIDPYYGGTSYNQKFKGRVTMTIDKST |
| | STVYMELSSLRSEDTAVYYCARMYHGNAFDYWGQGTTV |
| | TVSS (SEQ ID NO: 25) |
| Hu18B10-Hb | QVQLVQSGAEVKKPGASVKVSCKASGYSFTGYNMNWVR |
| | QAPGQGLEWMGNIDPYYGGTSYNQKFKGRVTLTIDKST |
| | STVYMELSSLRSEDTAVYYCARMYHGNAFDYWGQGTTV |
| | TVSS (SEQ ID NO: 27) |
| Hu18B10-Hc | QVQLVQSGAEVKKPGASVKVSCKASGYSFTGYNMNWVK |
| | QAPGQGLEWIGNIDPYYGGTSYNQKFKGRVTLTIDKST |
| | STVYMELSSLRSEDTAVYYCARMYHGNAFDYWGQGTTV |
| | TVSS (SEQ ID NO: 29) |
| 18B10-LC | DIVMTQSPDSLAVSLGERATINCKSSQNNKNYLAWYQQK |
| germline | PGQPPKLLIYWASTRESGVPDRFSGSGSGTDFTLTISSL |
| | QAEDVAVYYCQQYYSTP (SEQ ID NO: 24) |
| Hu18B10_La | DIVMTQSPDSLAVSLGERATINCKSSQSLLNSGNLKNYLT |
| | WYQQKPGQPPKLLIYWASTRKSGVPDRFSGSGSGTDFTLT |
| | ISSLQAEDVAVYYCQNDYSYPLTFGGGTKVEIK |
| | (SEQ ID NO: 26) |
| Hu18B10_Lb | DIVMTQSPDSLAVSLGERATMNCKSSQSLLNSGNLKNYLT |
| | WYQQKPGQPPKLLIYWASTRKSGVPDRFTGSGSGTDFTLT |
| | ISSLQAEDVAVYYCQNDYSYPLTFGGGTKVEIK |
| | (SEQ ID NO: 28) |

In certain embodiments, the humanized antibodies provided herein may comprise the heavy chain variable region fused to the constant region of human IgG1 isotype and the light chain variable region fused to the constant region of human kappa chain.

The humanized anti-CLDN18.2 antibody conjugates provided herein retained the specific binding affinity to CLDN18.2-expressing cell, and are at least comparable to, or even better than, the parent antibodies in that aspect. The humanized antibodies provided herein can also retain their functional interaction with CLDN18.2-expressing cells, such as NUGC4 cells, SNU-620 cell, SNU-601 cell, or KATOIII cell in that all antibodies can mediate cell killing by ADCC, CDC and induction of apoptosis induced by cross linking of the target at the tumor cell surface and direct inhibition of proliferation. In certain embodiments, the anti-CLDN18.2 antibodies and the fragments thereof provided herein further comprise an immunoglobulin constant region, optionally a constant region of human Ig, or optionally a constant region of human IgG. In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises Cκ or Cλ.

In certain embodiments, the anti-CLDN18.2 antibodies and the fragments thereof provided herein further comprise a constant region of human IgG1, IgG2, IgG3, or IgG4. In certain embodiments, the anti-CLDN18.2 antibodies and antigen-binding fragments thereof provided herein comprises a constant region of IgG1 isotype. In certain embodiments, the constant region of human IgG1 comprises SEQ ID NO: 49, or a homologous sequence having at least 80% (e.g. at least 85%, 90%, 95%, 96%, 97%, 98%, or 99%) sequence identity thereof.

Constant region of IgG1 isotype can induce effector functions such as ADCC or CDC. Effector functions of the anti-CLDN18.2 antibodies and the antigen-binding fragments thereof provided herein can lead to cytotoxicity to cells expressing CLDN18.2. Effector functions can be evaluated using various assays such as Fc receptor binding assay, C1q binding assay, and cell lysis assay, and any of the assays described above for determining ADCC or CDC.

It has been surprisingly found by the inventors that the anti-CLDN18.2 antibodies and the fragments thereof provided herein have high affinity to human CLDN18.2 medium-expressing cell lines (e.g. NUGC4 cell), low-expressing cell lines (e.g. SNU-620, SNU-601 and KATOIII cells). This distinguished from existing antibodies such as IMAB362, which fails to show specific or comparable binding to human CLDN18.2 low-expressing cells. The chimeric IgG1 antibody IMAB362 is an anti-human CLDN18.2 antibody developed by Ganymed Pharmaceuticals AG, having an amino acid sequence disclosed in U.S. patent application US2009169547A1 (IMB362's heavy and light chain variable region sequences are included herein as SEQ ID NO: 72 and SEQ ID NO: 73) and CAS number of 1496553-00-4. IMAB362 recognizes the first extracellular domain (ECD1) of CLDN18.2 and does not bind to any other claudin family member including the closely related splice variant 1 of Claudin 18 (CLDN18.1).

NUGC4 cell is a cell line established from paragastric lymph node from a cancer patient (see, Akiyama S et al, Jpn J Surg. 1988 July; 18(4):438-46). NUGC4 cell line is available from JCRB Cell Bank under the accession number JCRB0834.

SNU-601 cell and SNU-620 cell both are human stomach carcinoma cell lines established from ascites of cancer patients by Seoul National University (SNU) (KU J L et al, Cancer Res Treat. 2005 February; 37(1): 1-19; Park et al., Int J Cancer. 1997 Feb. 7; 70(4):443-449). SNU-601 cell and SNU-620 cell are available from Korean Cell Line Bank under the accession numbers of 00601 and 00620, respectively.

KATO III cell is a cell line derived from metastatic site of a gastric cancer patient (see, Sekiguchi M, et al. Jpn. J. Exp. Med. 48: 61-68, 1978.). KATO III cell line is available from ATCC under the accession number ATCC HTB-103.

In certain embodiments, the anti-CLDN18.2 antibodies, antigen-binding fragment thereof, and the anti-CLDN18.2 antibody conjugates provided herein specifically bind to a human CLDN18.2 expressing cell (e.g. NUGC4 cell line or KATOIII cell line) at a K_{D} value of no more than 2.5 nM (or no more than 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4 nM) as measured by KinExA assay. In certain embodiments, the anti-CLDN18.2 antibodies, antigen-binding fragment thereof, and the anti-CLDN18.2 antibody conjugates provided herein specifically bind to a human CLDN18.2 expressing cell at a Kd value no more than 80%, 70%, 60%, 50%, 40%, 30%, 20%, 15% of that of IMAB362, as measured by KinExA assay. In certain embodiments, the K_{D} value is determined with NUGC4 cell, KATOIII cell, SNU-601 cell, SNU-620 cell or a comparable cell thereof having a human CLDN18.2 protein expression level comparable to or no more than that of NUGC4 cell, KATOIII cell, SNU-601 cell, or SNU-620 cell. In certain embodiments, the K_{D} value is determined with a human CLDN18.2 high-expressing cell line or human CLDN18.2 medium-expressing cell line.

In certain embodiments, the anti-CLDN18.2 antibodies, antigen-binding fragment thereof, and the anti-CLDN18.2 antibody conjugates provided herein has an EC50 value for binding to a human CLDN18.2 (or a mouse CLDN18.2) expressing cell is no more than 70 μg/ml (or no more than 65, 60, 55, 50, 45, 40, 35, 30, 25, 20, 15, 12, or 10, 9, 8, 7, 6, 5, 4, 3, 2, or 1 μg/ml), as measured by flow cytometry assay. In certain embodiments, the anti-CLDN18.2 antibodies, antigen-binding fragment thereof, and the anti-CLDN18.2 antibody conjugates provided herein specifically bind to a human CLDN18.2 expressing cell at an EC50 value no more than 80%, 70%, 60%, 50%, 40%, 30%, 20%, 15%, 10%, 1%, or 0.1% of that of IMAB362, as measured by flow cytometry assay. In certain embodiments, the EC50 is determined with NUGC4 cell line, KATOIII cell line, SNU-601 cell line, SNU-620 cell line, or a comparable cell thereof having a human CLDN18.2 protein expression level comparable to or no more than that of NUGC4 cell line, KATOIII cell line, SNU-601 cell line, or SNU-620 cell line, for example, a human CLDN18.2 low-expressing cell line, or a human CLDN18.2 medium-expressing cell line. In certain embodiments, the EC50 is determined with a human CLDN18.2 high-expressing cell line.

In certain embodiments, the anti-CLDN18.2 antibodies, antigen-binding fragment thereof, and the anti-CLDN18.2 antibody conjugates provided herein has an EC50 value of no more than 5, 4, 3 or 2 μg/ml for binding to a human CLDN18.2 high-expressing cell line or human CLDN18.2 medium-expressing cell line.

In certain embodiments, the anti-CLDN18.2 antibodies, antigen-binding fragment thereof, and anti-CLDN18.2 antibody conjugates provided herein has an EC50 value for binding to NUGC4 cells of no more than 70 μg/ml (or no more than 65, 60, 55, 50, 45, 40, 35, 30, 25, 20, 15, 12, or 10, 9, 8, 7, 6, 5, 4, 3, 2, or 1 μg/ml), as measured by flow cytometry assay.

In certain embodiments, the anti-CLDN18.2 antibody conjugates provided herein do not bind to CLDN18.1 (e.g. human CLDN18.1 or mouse CLDN18.1).

In certain embodiments, the anti-CLDN18.2 antibodies, antigen-binding fragment thereof, and anti-CLDN18.2 antibody conjugates provided herein are capable of specifically binding to mouse CLDN18.2 (e.g. a cell expressing mouse CLDN18.2) at an EC50 value no more than 1.5 μg/ml as measured by Flow Cytometry. In certain embodiments, the anti-CLDN18.2 antibody conjugates provided herein bind to mouse CLDN18.2 at an EC50 of 0.1 μg/ml-1.5 μg/ml (e.g. 0.1 g/ml-1.2 μg/ml, 0.2 μg/ml-1 μg/ml, 0.5 μg/ml-1 μg/ml, 0.6 μg/ml-1 μg/ml, 0.6 μg/ml-0.8 μg/ml, or 0.67 μg/ml) as measured by Flow Cytometry.

In certain embodiments, the anti-CLDN18.2 antibodies provided herein and the anti-CLDN18.2 antibody conjugates provided herein conjugates provided herein are capable of inducing antibody-dependent cell-mediated cytotoxicity (ADCC) activity and/or CDC activity in cells expressing different levels of human CLDN18.2.

As used herein “antibody-dependent cell-mediated cytotoxicity” or “ADCC” refers to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. Lysis of the target cell is extracellular, requires direct cell-to-cell contact, and does not involve complement. ADCC can be viewed as a mechanism to directly induce a variable degree of immediate tumor destruction that leads to antigen presentation and the induction of tumor-directed T-cell responses. In vivo induction of ADCC is believed to lead to tumor-directed T-cell responses and host-derived antibody responses.

Methods for performing ADCC are known in the art. In general, target cells such as CLDN18.2-expressing cells are incubated with a range of concentrations of an anti-CLDN18.2 antibody, and after washing, effector cells such as Fc receptor expressing cells are added to allow ADCC to occur. Cytotoxicity or cell viability is determined at one time point several hours after the mixing of the target cells with effector cells, to quantify the level of ADCC. Cytotoxicity can be detected by the release of a label (e.g., radioactive substrates, fluorescent dyes or natural intracellular proteins such as lactate dehydrogenase (LDH)) from the lysed target cells. In another embodiment, cell viability is determined by the indicator (such as ATP) of metabolically active cells (see, for example, Crouch, S. P. et al. (1993) J. Immunol. Methods 160, 81-8), using a luciferase reporter gene which generates luminescent signal proportional to the number of living cells in culture (i.e. ADCC reporter assay). Examples of effector cells are NK cells, PBMCs, or FcγRIII-expressing cells.

“Complement dependent cytotoxicity” or “CDC” is another cell-killing method that can be directed by antibodies by lysing of a target in the presence of complement. IgM is the most effective isotype for complement activation. IgG1 and IgG3 are also both very effective at directing CDC via the classical complement-activation pathway. In this cascade, the formation of antigen-antibody complexes results in the uncloaking of multiple C1q binding sites in close proximity on the CH2 domains of participating antibody molecules such as IgG molecules (C1q is one of three subcomponents of complement C1) complexed with a cognate antigen. These uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a. The complement cascade ends in the formation of a membrane attack complex (MAC), which creates pores in the cell membrane that facilitate free passage of water and solutes into and out of the cell.

CDC activity can be determined by a method similar to that for ADCC activity, as discussed above, except that no effector cells are used presence of complement derived from human serum is required. Briefly, the antibody samples were serially diluted in assay medium, and incubated with target cells expressing CLDN18.2 in the presence of human serum complement. After the incubation, cytotoxicity or cell viability is determined by the release of a label from the lysed target cells, or by an indicator (such as ATP) of metabolically active cells. CellTiter-Glo reagent which assays for ATP in metabolically active cells can be used, and the extent of cell lysis can be quantified by measuring intensity of luminescence with a proper reader.

In certain embodiments, the ADCC or CDC induced cell death via the anti-CLDN18.2 antibodies, antigen-binding fragment thereof, and the anti-CLDN18.2 antibody conjugates provided herein can be determined by loss of membrane integrity as evaluated by uptake of propidium iodide (PI), trypan blue (see Moore et al. Cytotechnology 17:1-11 (1995)) or 7AAD can be assessed relative to untreated cells.

In certain embodiments, the anti-CLDN18.2 antibodies, antigen-binding fragment thereof, and the anti-CLDN18.2 antibody conjugates provided herein are capable of inducing complement dependent cytotoxicity (CDC) on a cell expressing human CLDN18.2 at an EC50 value of no more than 1 μg/ml (or no more than 0.9, 0.8, 0.7, 0.6, 0.5 μg/ml) as measured by cytotoxicity assay. In certain embodiments, the anti-CLDN18.2 antibodies and the fragments thereof provided herein are capable of inducing CDC on a cell expressing human CLDN18.2 at an EC50 value no more than 80%, 70%, 60%, 50%, 40%, 30%, 20%, 10%, or 5% of that of IMAB362, as measured by cytotoxicity assay. In certain embodiments, CDC is determined with human CLDN18.2 medium-expressing cell line or a human CLDN18.2 high-expressing cell line.

In certain embodiments, the anti-CLDN18.2 antibodies, antigen-binding fragment thereof, and the anti-CLDN18.2 antibody conjugates provided herein are capable of inducing antibody-dependent cell cytotoxicity (ADCC) on a cell expressing human CLDN18.2 at an EC50 value of no more than 2 μg/ml (or no more than 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1 μg/ml) as measured by an ADCC reporter assay. In certain embodiments, the anti-CLDN18.2 antibodies, antigen-binding fragment thereof, and the anti-CLDN18.2 antibody conjugates provided herein induce ADCC on a cell expressing human CLDN18.2 at an EC50 value no more than 80%, 70%, 60%, 50%, 40%, 30%, 20%, 10%, 5%, or 1% of that of IMAB362, or at a total ADCC capacity (e.g. as indicated by the maximum level of ADCC activity observed in a plot of antibody concentration versus ADCC activity level) at least 120%, 150%, 180%, or²⁰⁰% of that of IMAB362, as measured by an ADCC reporter assay. In certain embodiments, the ADCC is determined with NUGC4 cell line, KATOIII cell line, SNU-601 cell line, SNU-620 cell line or a comparable cell thereof having a human CLDN18.2 protein expression level comparable to or no more than that of NUGC4 cell line, KATOIII cell line, SNU-601 cell line, or SNU-620 cell line, for example, a human CLDN18.2 medium-expressing cell line or a human CLDN18.2 low-expressing cell line.

In certain embodiments, the anti-CLDN18.2 antibodies, antigen-binding fragment thereof, and the anti-CLDN18.2 antibody conjugates provided herein are capable of inducing ADCC on NUGC4 cells at an EC50 value of no more than 2 μg/ml (or no more than 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1 μg/ml) as measured by an ADCC reporter assay.

In certain embodiments, the anti-CLDN18.2 antibodies, antigen-binding fragment thereof, and the anti-CLDN18.2 antibody conjugates provided herein binds to an epitope comprising at least one or more (e.g. one, two, three or more) of amino acid residues at positions D28, W30, V43, N45, Y46, L49, W50, R51, R55, E56, F60, E62, Y66, L72, L76, V79 and R80 of human CLDN18.2 having the amino acid sequence of SEQ ID NO: 30.

The term “epitope” as used herein refers to the specific group of atoms or amino acids on an antigen to which an antibody binds. An epitope can include specific amino acids, sugar side chains, phosphoryl or sulfonyl groups that directly contact an antibody. Those skilled in the art will recognize that it is possible to determine, without undue experimentation, if an antibody binds to the same or overlapping or adjacent epitope as the antibody of present disclosure (e.g., hybridoma/chimeric or humanized antibodies 7C12, 11F12, 26G6, 59A9, 18B10 and any of the chimeric and humanized variant thereof provided herein) by ascertaining whether the two competes for binding to a CLDN18.2 antigen polypeptide.

The term “compete for binding” as used herein with respect to two antigen-binding proteins (e.g. antibodies), means that one antigen-binding protein blocks or reduces binding of the other to the antigen (e.g., human/mouse CLDN18.2), as determined by a competitive binding assay. Competitive binding assays are well known in the art, include, for example, direct or indirect radioimmunoassay (RIA), direct or indirect enzyme immunoassay (EIA), and sandwich competition assay (see, e.g., Stahli et al., 1983, Methods in Enzymology 9:242-253). Typically, such an assay involves the use of purified antigen bound to a solid surface or cells bearing the antigen, an unlabelled test antibody and a labeled reference antibody. Competitive inhibition is measured by determining the amount of label bound to the solid surface or cells in the presence of the test antibody. Usually the test antibody is present in excess. If two antibodies competes for binding to the CLDN18.2, then the two antibodies bind to the same or overlapping epitope, or an adjacent epitope sufficiently proximal to the epitope bound by the other antibody for steric hindrance to occur. Usually, when a competing antibody is present in excess, it will inhibit (e.g., reduce) specific binding of a test antibody to a common antigen by at least 50-55%, 55-60%, 60-65%, 65-70%, 70-75% 75-80%, 80-85%, 85-90% or more.

In certain embodiments, the epitope or the amino acid residue in the epitope bound by an antibody can be determined by mutating specific residues in the antigen, i.e., CLDN18.2. If an antibody binds to a mutant CLDN18.2 having an amino acid residue mutated, for example to alanine, at significantly reduced level relative to its binding to wild-type CLDN18.2, then this would indicate that the mutated residue is directly involved in the binding of the antibody to CLDN18.2 antigen, or is in close proximity to the antibody when it is bound to the antigen. Such a mutated residue is considered to be within the epitope, and the antibody is considered to specifically bind to an epitope comprising the residue. A significantly reduced level in binding as used herein, means that the binding affinity (e.g. EC50, Kd, or binding capacity) between the antibody and the mutant CLDN18.2 is reduced by greater than 10%, 20%, 30%, 40%, 50%, 60%, 70%, 80%, 90%, or more, relative to the binding between the antibody and a wild type CLDN18.2. Such a binding measurement can be conducted using any suitable methods known in the art and disclosed herein, for example, without limitation, KinExA assay, and flow cytometry.

In certain embodiments, the anti-CLDN18.2 antibodies, antigen-binding fragment thereof, and the anti-CLDN18.2 antibody conjugates provided herein exhibit significantly lower binding for a mutant CLDN18.2 in which a residue in a wild-type CLDN18.2 is substituted with alanine, and the residue is selected from the group consisting of: D28, W30, V43, N45, Y46, L49, W50, R51, R55, E56, F60, E62, Y66, L72, L76, V79 and R80 of human CLDN18.2. In certain embodiments, the residue is E56. In certain embodiments, the residue is selected from the group consisting of: W30, L49, W50, R55, and E56. In certain embodiments, the residue is selected from the group consisting of: T41, N45, Y46, R51, F60, E62, and R80. In certain embodiments, the residue is selected from the group consisting of: D28, V43, N45, Y46, Y66, L72, L76, and V79.

In certain embodiments, the anti-CLDN18.2 antibodies, antigen-binding fragment thereof, and the anti-CLDN18.2 antibody conjugates provided herein exhibit at least 80%, 90%, 95% or 99% or more reduction in binding for a mutant CLDN18.2 comprising E56A of human CLDN18.2, relative to the binding between the antibody and a wild type CLDN18.2.

In certain embodiments, the anti-CLDN18.2 antibodies, antigen-binding fragment thereof, and the anti-CLDN18.2 antibody conjugates provided herein exhibit at least 50%, 60%, 70%, 80%, or 90% reduction in binding for a mutant CLDN18.2 comprising one or more mutated residue selected from the group consisting of: W30A, L49A, W50A, R55A, and E56A of human CLDN18.2, relative to the binding between the antibody and a wild type CLDN18.2.

In certain embodiments, the anti-CLDN18.2 antibodies, antigen-binding fragment thereof, and the anti-CLDN18.2 antibody conjugates provided herein exhibit at least 30%, 35%, 40%, 45%, or 50% reduction in binding for a mutant CLDN18.2 comprising one or more mutated residue selected from the group consisting of: D28, V43, N45, Y46, Y66, L72, L76, and V79 of human CLDN18.2, relative to the binding between the antibody and a wild type CLDN18.2.

In certain embodiments, the anti-CLDN18.2 antibodies, antigen-binding fragment thereof, and the anti-CLDN18.2 antibody conjugates provided herein exhibit at least 10%, 15%, 20%, 25%, or 30% reduction in binding for a mutant CLDN18.2 comprising one or more mutated residue selected from the group consisting of: T41A, N45A, Y46A, R51A, F60A, E62A, and R80A of human CLDN18.2, relative to the binding between the antibody and a wild type CLDN18.2.

In certain embodiments, the anti-CLDN18.2 antibodies, antigen-binding fragment thereof, and the anti-CLDN18.2 antibody conjugates provided herein do not bind to A42, and/or N45.

In certain embodiments, the anti-CLDN18.2 antibodies, antigen-binding fragment thereof, and the anti-CLDN18.2 antibody conjugates provided herein are capable of binding to the epitope provided herein, and inducing ADCC or CDC activity in a human CLDN18.2 medium-expressing cell line or a human CLDN18.2 low-expressing cell line.

ii. Antibody Variants

The anti-CLDN18.2 antibodies and antigen-binding fragments thereof in the anti-CLDN18.2 antibody conjugates provided herein also encompass various types of variants of the antibody sequences provided herein.

In certain embodiments, the variants comprise one or more modification(s) or substitution(s) in 1, 2, or 3 CDR sequences as provided in Table 1, in one or more FR sequences, in the heavy or light chain variable region sequences provided herein, and/or in the constant region (e.g., Fc region). Such antibody variants retain specific binding affinity to CLDN18.2 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or increased effector function(s), improved FcRn receptor binding, increased pharmacokinetic half-life, pH sensitivity, and/or compatibility to conjugation (e.g., one or more introduced cysteine residues), to name a few.

A parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

### a) Affinity Variant

An affinity variant retain specific binding affinity to CLDN18.2 of the parent antibody, or even have improved CLDN18.2 specific binding affinity over the parent antibody. Various methods known in the art can be used to achieve this purpose. For example, a library of antibody variants (such as Fab or scFv variants) can be generated and expressed with phage display technology, and then screened for the binding affinity to human CLDN18.2. For another example, computer software can be used to virtually simulate the binding of the antibodies to human CLDN18.2, and identify the amino acid residues on the antibodies which form the binding interface. Such residues may be either avoided in the substitution so as to prevent reduction in binding affinity, or targeted for substitution to provide for a stronger binding.

In certain embodiments, at least one (or all) of the substitution(s) in the CDR sequences, FR sequences, or variable region sequences comprises a conservative substitution. A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among residues with neutral hydrophilic side chains (e.g., Cys, Ser, Thr, Asn and Gln), among residues with acidic side chains (e.g., Asp, Glu), among amino acids with basic side chains (e.g., His, Lys, and Arg), or among residues with aromatic side chains (e.g., Trp, Tyr, and Phe). As known in the art, conservative substitution usually does not cause significant change in the protein conformational structure, and therefore could retain the biological activity of a protein.

In certain embodiments, the antibody or antigen-binding fragment provided herein comprises one or more amino acid residue substitutions in one or more CDR sequences, and/or one or more FR sequences. In certain embodiments, an affinity variant comprises no more than 10, 9, 8, 7, 6, 5, 4, 3, 2, or 1 substitutions in one or more of the CDR sequences and/or FR sequences in total.

In certain embodiments, the anti-CLDN18.2 antibodies and antigen-binding fragments thereof comprise 1, 2, or 3 CDR sequences having at least 80% (e.g., at least 85%, 88%, 90%, 91%, 92%, 93%, 94%, 95%, 96%, 97%, 98%, 99%) sequence identity to that (or those) listed in Table 1, and in the meantime retain the binding affinity to CLDN18.2 at a level similar to or even higher than its parental antibody.

In certain embodiments, the anti-CLDN18.2 antibodies and antigen-binding fragments thereof comprise one or more variable region sequences having at least 80% (e.g., at least 85%, 88%, 90%, 91%, 92%, 93%, 94%, 95%, 96%, 97%, 98%, 99%) sequence identity to that (or those) of SEQ ID NOs: 23-29 and 37-48, and in the meantime retain the binding affinity to CLDN18.2 at a level similar to or even higher than its parent antibody. In some embodiments, a total of 1 to 10 amino acids have been substituted, inserted, or deleted in a sequence selected from SEQ ID NOs: 25-29 and 37-48. In some embodiments, the substitutions, insertions, or deletions occur in regions outside the CDRs (i.e., in the FRs).

### b) Glycosylation Variant

The anti-CLDN18.2 antibodies and antigen-binding fragments in the anti-CLDN18.2 antibody conjugates provided herein also encompass a glycosylation variant, which can be obtained to either increase or decrease the extent of glycosylation of the antibody or antigen binding fragment. The term “glycosylation” as used herein, refers to enzymatic process that attaches glycans such as fucose, xylose, mannose, or GlcNAc phosphoserine glycan to proteins, lipids, or other organic molecules. Depending on the carbon linked to the glycan, glycosylation can be divided into five classes including: N-linked glycosylation, O-linked glycosylation, phospho-glycosylation, C-linked glycosylation, and glypiation.

Glycosylation of antibodies is typically N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine.

In certain embodiments, the anti-CLDN18.2 antibodies and antigen-binding fragments provided herein encompass a glycosylation variant having improved effector functions such as ADCC or CDC.

In certain embodiments, the antibody or antigen-binding fragment thereof provided herein is afucosylated. The term “afucosylation,” or “afucosylated,” refers to the reduced or eliminated core-fucose on the N-glycan attached to the antibody. The majority glycans of human IgG antibodies are known as G0, G1 and G2, which are complex biantennary molecules with core fucose residue carrying zero, one or two terminal galactose.

Afucosylated antibody variants can be made using methods known in the art, for example, as described in US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004).

In certain embodiments, the antibody glycosylation variant is afucosylated at Asn297 site of CH2 region in Fc of the antibody. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies.

In certain embodiments, the antibody glycosylation variants can be obtained by, for example, removal of a native glycosylation site (e.g. by N297A substitution), such that tripeptide sequences for N-linked glycosylation sites or serine or threonine residues for O-linked glycosylation sites no longer present in the antibody or Fc sequence. Alternatively, in certain embodiments, antibody glycosylation variants can be obtained by producing the antibody in a host cell line that is defective in adding the selected sugar group(s) to the mature core carbohydrate structure in the antibody.

### c) Cysteine-Engineered Variant

The anti-CLDN18.2 antibodies and antigen-binding fragments in the anti-CLDN18.2 antibody conjugates provided herein also encompass a cysteine-engineered variant, which comprises one or more introduced free cysteine amino acid residues.

A free cysteine residue is one which is not part of a disulfide bridge. A cysteine-engineered variant is useful for conjugation with, for example a cytotoxic and/or imaging compound, a label, or a radioisoptype among others, at the site of the engineered cysteine, through for example a maleimide or haloacetyl. Methods for engineering antibodies or antigen-binding fragments to introduce free cysteine residues are known in the art, see, for example, WO2006/034488.

### d) Fc Variants

The anti-CLDN18.2 antibodies and antigen-binding fragments in the anti-CLDN18.2 antibody conjugates provided herein also encompass an Fc variant, which comprises one or more amino acid residue modifications or substitutions at its Fc region and/or hinge region.

In certain embodiments, the anti-CLDN18.2 antibody conjugates provided herein comprise constant region comprising one or more amino acid residue substitutions or modifications conferring increased CDC or ADCC relative to wild-type constant region. Certain amino acid residues at CH2 domain of the Fc region can be substituted to provide for enhanced ADCC activity, for example, by enhancing the affinity of the Fc domain to FcγRIIIA. Methods of altering ADCC activity by antibody engineering have been described in the art, see for example, Shields R L. et al., J Biol Chem. 2001. 276(9): 6591-604; Idusogie E E. et al., J Immunol. 2000.164(8):4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O., et al., Mol Cancer Ther. 2008, 7(8): 2517-27; Shields R. L. et al, J. Biol. Chem, 2002, 277: 26733-26740; Shinkawa T. et al, J. Biol. Chem, 2003, 278: 3466-3473.

In certain embodiments, the anti-CLDN18.2 antibody conjugates provided herein comprise one or more amino acid substitution(s) that alters Complement Dependent Cytotoxicity (CDC), for example, by improving or diminishing C1q binding and/or Complement Dependent Cytotoxicity (CDC) (see, for example, WO99/51642; Duncan & Winter Nature 322:738-40 (1988); U.S. Pat. Nos. 5,648,260; 5,624,821; and WO94/29351 concerning other examples of Fc region variants.

In certain embodiments, the constant region of the antibodies or antigen-binding fragments thereof provided herein comprises one or more amino acid residue substitutions relative to SEQ ID NO: 49 (i.e. the wild-type sequence), selected from the group consisting of: L235V, F243L, R292P, Y300L, P396L, or any combination thereof. In certain embodiments, the constant region comprises the sequence of SEQ ID NO: 51.

In certain embodiments, the anti-CLDN18.2 antibody conjugates provided herein comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al, Structure, 6(1): 63-73, 1998; Kontermann, R. et al, Antibody Engineering, Volume 1, Chapter 27: Engineering of the Fc region for improved PK, published by Springer, 2010; Yeung, Y. et al, Cancer Research, 70: 3269-3277 (2010); and Hinton, P. et al, J. Immunology, 176:346-356 (2006).

iii. Antigen-Binding Fragments

The anti-CLDN18.2 antibody conjugates provided herein may also comprise anti-CLDN18.2 antigen-binding fragments. Various types of antigen-binding fragments are known in the art and can be developed based on the anti-CLDN18.2 antibodies provided herein, including for example, the exemplary antibodies whose CDR sequences are shown in Tables 1, and their different variants (such as affinity variants, glycosylation variants, Fc variants, cysteine-engineered variants and so on).

In certain embodiments, an anti-CLDN18.2 antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. Coli (e.g., for Fab, Fv and ScFv antibody fragments), screening from a phage display library as discussed above (e.g., for ScFv), and chemical coupling of two Fab′-SH fragments to form F(ab′)₂ fragments (Carter et al., Bio/Technology 10:163-167 (1992)). Other techniques for the production of antibody fragments will be apparent to a skilled practitioner.

In certain embodiments, the antigen-binding fragment is a scFv. Generation of scFv is described in, for example, WO 93/16185; U.S. Pat. Nos. 5,571,894; and 5,587,458. scFv may be fused to an effector protein at either the amino or the carboxyl terminus to provide for a fusion protein (see, for example, Antibody Engineering, ed. Borrebaeck).

In certain embodiments, the anti-CLDN18.2 antibodies and antigen-binding fragments thereof in the anti-CLDN18.2 antibody conjugates provided herein are bivalent, tetravalent, hexavalent, or multivalent. The term “valent” as used herein refers to the presence of a specified number of antigen binding sites in a given molecule. As such, the terms “bivalent”, “tetravalent”, and “hexavalent” denote the presence of two binding site, four binding sites, and six binding sites, respectively, in an antigen-binding molecule. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Bispecific Antibodies

In certain embodiments, the anti-CLDN18.2 antibody conjugates provided herein are bispecific. The term “bispecific” as used herein encompasses molecules having more than two specificity and molecules having more than two specificity, i.e. multispecific. In certain embodiments, the bispecific antibodies and antigen-binding fragments thereof provided herein is capable of specifically binding to a first and a second epitopes of CLDN18.2, or capable of specifically binding to CLDN18.2 and a second antigen. In certain embodiments, the first epitope and the second epitopes of CLDN18.2 are distinct from each other or non-overlapping. In certain embodiments, the bispecific antibodies and antigen-binding fragments thereof can bind to both the first epitope and the second epitope at the same time. In certain embodiments, the second antigen is different from CLDN18.2.

In certain embodiments, the second antigen comprises a tumor antigen. “Tumor antigen” as used herein refers to tumor specific antigens (e.g. those unique to tumor cells and normally not found on non-tumor cells), tumor-associated antigens (e.g. found in both tumor and non-tumor cells but expressed differently in tumor cells), and tumor neo-antigens (e.g. that are expressed in cancer cells because of somatic mutations that change the protein sequence or create fusion proteins between two unrelated sequences).

### Methods of Preparation

In another aspect, the present disclosure provides a method of preparing the anti-CLDN18.2 antibody conjugate provided herein, comprising reacting an anti-CLDN18.2 antibody or an antigen-binding fragment thereof with an iodide compound labeled with ¹²⁴, ¹²³I or ¹³¹I, in the presence of an enzymatic or chemical oxidant.

In some embodiments, the chemical oxidant is N-bromosuccinimide, Iodogen, or Chloramine-T.

In another aspect, the present disclosure provides a method of preparing the anti-CLDN18.2 antibody conjugate provided herein, comprising conjugating an anti-CLDN18.2 antibody or an antigen-binding fragment thereof with a chelator to obtain a chelator-antibody conjugate, and reacting the chelator-antibody conjugate with a metallic radionuclide, for example, ⁶⁴Cu, or ⁸⁹Zr.

In some embodiments, the chelator comprises DFO (derferoxamine), DOTA (1,4,7,10-tetraazacyclododecane-1,4,7,10-tetracetic acid), DTPA (NR-diethylenetriaminepentacetic acid), NOTA (1,4,7-triazacyclononane-1,4,7-acetic acid), TRITA (1,4,7,10-tetraazacyclotridecane-N,N′,N″,N′″-tetraacetic acid); TETA (1,4,8,11-tetraazacyclotetradecane-N,N′,N″,N′″-tetraacetic acid); and HETA (1,5,9,13-tetraazacyclohexadecane-N,N′,N″,N′″-tetraacetic acid), EDTA (ethylenediaminetetraacetic acid), NETA({4-[2-(bis-carboxymethylamino)-ethyl]-7-carboxymethyl-[1,4,7]triazonan-1-yl}-acetic acid), TACN-TM (N,N′,N″, tris(2-mercaptoethyl)1,4,7-triazacyclononane), TRAP (1,4,7-triazacyclononane-1,4,7-tris[methyl(2-carboxyethyl)phosphinic acid]), CP256,PCTA (3,6,9,15-tetraazabicyclo[9.3.1]pentadeca-1(15), 11,13-triene-3,6,9,-triacetic acid), porphyrins, polyamines, crown ethers, bis-thiosemicarbazones, polyoximes, and derivatives thereof.

### Pharmaceutical Composition

The present disclosure further provides pharmaceutical compositions comprising the anti-CLDN18.2 antibody conjugates and one or more pharmaceutically acceptable carriers. In certain embodiments, the pharmaceutical compositions provided herein are suitable for parenteral administration, for example, suitable for bolus, intravenous, or intra-tumor injection. In certain embodiments, the pharmaceutical compositions are in a unit dosage injectable form.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibody conjugates as disclosed herein and one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-CLDN18.2 antibody conjugate or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Methods of Treating and Diagnosing CLDN18.2 Associated Conditions

The anti-CLDN18.2 antibody conjugates provided herein can be useful in methods for diagnosing or treating CLDN18.2 associated conditions. In certain embodiments, the CLDN18.2 associated condition is cancer. In certain embodiments, the CLDN18.2 associated condition is CLDN18.2-expressing cancer.

In certain embodiments, the cancer is selected from gastric cancer, lung cancer, bronchial cancer, bone cancer, liver and bile duct cancer, pancreatic cancer, breast cancer, liver cancer, ovarian cancer, testicle cancer, kidney cancer, bladder cancer, head and neck cancer, spine cancer, brain cancer, cervix cancer, uterine cancer, endometrial cancer, colon cancer, colorectal cancer, rectal cancer, anal cancer, esophageal cancer, gastrointestinal cancer, skin cancer, prostate cancer, pituitary cancer, stomach cancer, vagina cancer, thyroid cancer, glioblastoma, astrocytoma, melanoma, myelodysplastic syndrome, sarcoma, teratoma, and adenocarcinoma.

Examples of cancers include but are not limited to, non-small cell lung cancer (squamous/nonsquamous), small cell lung cancer, renal cell cancer, colorectal cancer, colon cancer, ovarian cancer, breast cancer (including basal breast carcinoma, ductal carcinoma and lobular breast carcinoma), pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, melanoma, myelomas, mycoses fungoids, merkel cell cancer, hepatocellular carcinoma (HCC), fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, lymphoid malignancy, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, hepatoma, bile duct carcinoma, choriocarcinoma, Wilms' tumor, cervical cancer, testicular tumor, seminoma, classical Hodgkin lymphoma (CHL), primary mediastinal large B-cell lymphoma, T-cell/histiocyte-rich B-cell lymphoma, acute lymphocytic leukemia, acute myelocytic leukemia, acute myelogenous leukemia, chronic myelocytic (granulocytic) leukemia, chronic myelogenous leukemia, chronic lymphocytic leukemia, polycythemia vera, mast cell derived tumors, EBV-positive and -negative PTLD, and diffuse large B-cell lymphoma (DLBCL), plasmablastic lymphoma, extranodal NK/T-cell lymphoma, nasopharyngeal carcinoma, HHV8-associated primary effusion lymphoma, non-Hodgkin's lymphoma, multiple myeloma, Waldenstrom's macroglobulinemia, heavy chain disease, myelodysplastic syndrome, hairy cell leukemia and myelodysplasia, primary CNS lymphoma, spinal axis tumor, brain stem glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma.

“CLDN18.2-expressing cancer” as used herein refers to any cancer or tumor involving cancer cells expressing CLDN18.2. Examples of CLDN18.2-expressing cancer include gastric cancer, esophageal cancer, pancreatic cancer, lung cancer such as non-small cell lung cancer (NSCLC) and small cell lung cancer (SCLC), ovarian cancer, colon cancer, colorectal cancer, gastrointestinal stromal tumors (GIST), gastrointestinal carcinoid tumors, rectal cancer, anal cancer, bile duct cancer, small intestine cancer, appendix cancer; prostate cancer, renal cancer (e.g., renal cell carcinoma), hepatic cancer, head-neck cancer, and cancer of the gallbladder and metastases thereof, for example, gastric cancer metastasis such as Krukenberg tumors, peritoneal metastasis and lymph node metastasis.

In certain embodiments, the CLDN18.2-expressing cancer can be an adenocarcinoma, for example, an advanced adenocarcinoma. In certain embodiments, the cancer is selected from adenocarcinomas of the stomach, the esophagus, the pancreatic duct, the bile ducts, the lung and the ovary. In certain embodiments, the CLDN18.2-expressing cancer comprises a cancer of the stomach, a cancer of the esophagus, in particular the lower esophagus, a cancer of the eso-gastric junction and gastroesophageal cancer.

Methods of Detecting, Visualizing, Diagnosing, Staging and Monitoring CLDN18.2 Associated Conditions

The antibody conjugates provided herein are useful in detecting CLDN18.2 expression, in particular, in detecting in vivo in a non-invasive manner.

The antibody conjugates provided herein are also useful in visualizing, diagnosing, staging, and monitoring CLDN18.2 associated conditions. In certain embodiments, the CLDN18.2 associated condition is cancer. In certain embodiments, the CLDN18.2 associated condition is CLDN18.2-expressing cancer.

The antibody radionuclide conjugates provided herein can be used in vivo to provide information about (i) expression of CLDN18.2, (ii) distribution of CLDN18.2, and (iii) change in expression or distribution of CLDN18.2. Compared with currently available diagnostic methods for CLDN18.2 such as immunohistochemistry (IHC), the antibody radionuclide conjugates provided herein are advantageous in non-invasive treatment, quantifiable, whole body assessment, and repetitive dosing and assessment at multiple time points.

The anti-CLDN18.2 antibody conjugates provided herein provided herein may be used with an in vivo nuclear imaging modality to visualize the target cells within the topography of the subject's body. In addition to diagnosing a cancer associated with CLDN18.2 expression, the anti-CLDN18.2 antibody radionuclide conjugates provided herein may also be used to stage, and monitor cancer progression according to methods provided herein.

Suitable methods of in vivo nuclear imaging that may be used in accordance with the methods described herein include, but are not limited to, positron emission tomography (PET), PET/CT combination imager, and single photon emission computed tomography (SPECT).

In one aspect, the present disclosure provides a method of obtaining an image of a site of interest in a subject, the method comprising the steps of:
a) administering to the subject an effective amount of the antibody conjugate provided herein and/or the pharmaceutical composition provided herein; and
b) subjecting the site of interest of the subject to positron emission tomography (PET) or SPECT;
c) identifying a detectable signal from the radionuclide in the subject;
d) generating an image of the detectable signal, thereby obtaining an image of the site of interest in the subject.

In some embodiments, the site of interest is a site expressing or suspected of expressing claudin 18.2.

In some embodiments, the site of interest has or is suspected of having tumor. In some embodiments, the site of interest can be whole body, torso, head, or limbs.

In another aspect, the present disclosure provides a method of detecting or visualizing claudin 18.2 expression in a subject in a non-invasive manner, comprising the steps of:
a) administering to the subject an effective amount of the antibody conjugate provided herein and/or the pharmaceutical composition provided herein; and
b) subjecting the subject to positron emission tomography (PET) or SPECT;
c) identifying a detectable signal from the radionuclide in a site of interest of the subject;
d) determining or visualizing claudin18.2 expression in the site of interest of the subject based on the identified detectable signal.

In some embodiments, the CLDN18.2 antibody conjugates provided herein can be injected intravenously into the subject. The subject may be scanned by PET after a certain period of time post injection.

In certain embodiments, the method further comprising subjecting the subject to X-ray Computed Tomography (CT), to obtain an image of the section of the site of the body under investigation. In certain embodiments, a merged image of CT and PET can be obtained to show the CLDN18.2 expression and distribution on top of a body reference frame.

In some embodiments, the method further comprises administering a therapeutically effective amount of anti-claudin 18.2 therapy to the subject identified as having claudin18.2 expression in the site of interest.

An anti-claudin 18.2 therapy can be any therapeutic agents targeting CLDN18.2, including without limitation, anti-CLDN18.2 antibodies, anti-CLDN18.2 antibody-drug conjugates, small molecule compounds targeting CLDN18.2, nucleic acid targeting CLDN18.2, or cell therapy targeting CLDN18.2.

In some embodiments, the method further comprises determining distribution of claudin18.2 expression in the site of interest of the subject based on the identified detectable signal.

In some embodiments, the method further comprises determining heterogeneity of claudin18.2 expression in the site of interest of the subject.

In some embodiments, the site of interest is tumor.

In another aspect, the present disclosure provides a method of monitoring therapeutic efficacy, responsiveness to treatment, or development of resistance or recurrence, or metastasis, in a subject in a non-invasive manner, wherein the subject has received treatment for a therapeutic period, comprising the steps of:
a) administering to the subject an effective amount of the antibody conjugate provided herein and/or the pharmaceutical composition provided herein; and
b) subjecting the subject to positron emission tomography (PET) or SPECT;
c) identifying a detectable signal from the radionuclide in a site of interest of the subject;
d) determining post-treatment claudin18.2 expression in a site of interest of the subject based on the identified detectable signal;
e) comparing the post-treatment claudin18.2 expression level or distribution, with a baseline claudin18.2 expression level or distribution, respectively, obtained from the subject before the therapeutic period, to determine post-treatment change in the claudin18.2 expression level or distribution in the subject; and
f) determining the therapeutic efficacy, responsiveness to treatment, or development of resistance or recurrence, or metastasis based on the change determined in step (e).

In some embodiments, the baseline claudin18.2 expression level or distribution is determined using a similar method.

In some embodiments, the baseline claudin18.2 expression level or distribution is determined before the therapeutic period (i.e. pre-treatment), by administering to the subject an effective amount of the antibody conjugate provided herein and/or the pharmaceutical composition provided herein; and subjecting the subject to positron emission tomography (PET) or SPECT, identifying a detectable signal from the radionuclide in a site of interest of the subject, and determining the baseline claudin18.2 expression in a site of interest of the subject based on the identified detectable signal.

In some embodiments, an increased level or spread of claudin 18.2 expression indicates reduced therapeutic efficacy, reduced responsiveness to treatment, or presence of resistance or presence of recurrence. In some embodiments, metastasis can be indicated by spread of claudin 18.2 expression to a site where claudin 18.2 expression is previously not detectable.

In some embodiments, the subject has cancer.

In some embodiments, the site of interest for determination of baseline claudin 18.2 expression is the same as, or at least comparable to, the site of interest for determination of post-treatment claudin 18.2 expression. The site of interest is comparable to another site of interest when both are of the same type of tissue or located within the same lesion.

In some embodiments, the site of interest for determination of baseline claudin 18.2 expression may also be different from the site of interest for determination of post-treatment claudin 18.2 expression.

### Methods of Treatment

In another aspect, methods are provided to treat a disease or condition in a subject that would benefit from modulation of CLDN18.2 activity, comprising administering a therapeutically effective amount of the CLDN18.2 antibody conjugates and/or the pharmaceutical composition provided herein to a subject in need thereof.

In certain embodiments, the disease or condition is a CLDN18.2 related disease or condition. In some embodiment, the CLDN18.2-related disease or condition is cancer. In some embodiment, the CLDN18.2-related disease or condition is CLDN18.2-expressing cancer.

In certain embodiments, the CLDN18.2 antibody conjugates comprises a therapeutic radionuclide.

In certain embodiments, therapeutic radionuclide is selected from the group consisting of ¹¹¹In, ^{111m}In, ¹⁷⁷Lu, ²²Bi, ²¹³Bi, ²¹¹At, ⁶²Cu, ⁶⁴Cu, ⁶⁷Cu, ⁹Y ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, 99Mo, ¹⁰⁵Rh, ¹⁰⁹Pd, ¹⁴³Pr, ¹⁴⁹Pm, ¹⁶⁹Er, ¹⁹⁴Ir ¹⁹⁸Au, ¹⁹⁹Au, ¹⁹⁹Au, and ²¹¹Pb.

In certain embodiments, therapeutic radionuclide is selected from the group consisting of ¹³¹I, ¹⁷⁷Lu, ¹⁵³Sm, ²²³Ra, ⁸⁹Sr, ⁹⁰Y, ²¹³Bi, ²¹²Pb, ¹⁶⁶Ho.

The therapeutically effective amount of an CLDN18.2 antibody conjugates as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements. The CLDN18.2 antibody conjugates disclosed herein may be administered by any route known in the art, such as for example parenteral (e.g., subcutaneous, intraperitoneal, intravenous, including intravenous infusion, intramuscular, or intradermal injection).

In some embodiments, the method further comprises administering a therapeutically effective amount of the CLDN18.2 antibody conjugates provided herein to the subject.

In certain embodiments, the subject is identified as having a CLDN18.2-expressing cancer cell. The presence and/or expression level of CLDN18.2 on a cancer cell can be determined by various methods known in the art, such as quantitative fluorescence cytometry, immunohistochemistry (IHC), qPCR, reverse transcriptase PCR, microarray, SAGE, FISH, and the like. The presence and/or expression level of CLDN18.2 in the subject may also be determined based on methods provided herein.

In certain embodiments, the subject has been diagnosed as having CLDN18.2-expressing cancer using the methods provided herein.

In certain embodiments, the subject has been diagnosed as having CLDN18.2-expressing cancer using the antibody conjugates provided herein comprising a diagnostic radionuclide as provided herein.

In some embodiments, the CLDN18.2 antibody conjugates disclosed herein may be administered alone or in combination with one or more additional therapeutic means or agents. For example, the CLDN18.2 antibody conjugates disclosed herein may be administered in combination with a second therapeutic agent, for example, a chemotherapeutic agent, an anti-cancer drug, radiation therapy, an immunotherapy, anti-angiogenesis agent, a targeted therapy, a cellular therapy, a gene therapy, a hormonal therapy, palliative care, surgery for the treatment of cancer (e.g., tumorectomy), or one or more anti-emetics or other treatments for complications arising from chemotherapy.

In certain of these embodiments, the CLDN18.2 antibody conjugates as disclosed herein that is administered in combination with one or more additional therapeutic agents, and in certain of these embodiments the CLDN18.2 antibody conjugates and the additional therapeutic agent(s) may be administered as part of the same pharmaceutical composition. However, a CLDN18.2 antibody conjugates administered “in combination” with another therapeutic agent does not have to be administered simultaneously with or in the same composition as the agent. The the CLDN18.2 antibody conjugate administered prior to or after another agent is considered to be administered “in combination” with that agent as the phrase is used herein, even if the CLDN18.2 antibody conjugate and second agent are administered via different routes. Where possible, additional therapeutic agents administered in combination with the antibodies or antigen-binding fragments disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

### Kits

In another aspect, the present disclosure provides kits comprising the anti-CLDN18.2 antibody conjugate provided herein or the composition provided herein.

In some embodiments, the kits may be useful in detection of presence or amount or distribution of CLDN18.2 in a subject, or may be useful in the methods of diagnosis provided herein.

In some embodiments, the kits may be useful in treatment of a CLDN18.2-expressing cancer in a subject, or may be useful in the methods of treatment provided herein.

In some embodiments, the kits comprise a first composition comprising a first anti-CLDN18.2 antibody conjugate provided herein comprising a therapeutic radionuclide disclosed herein, and a second composition comprising a second anti-CLDN18.2 antibody conjugate provided herein comprising a diagnostic radionuclide disclosed herein. In some embodiments, the therapeutic radionuclide is selected from the group consisting of ¹¹¹In, ^{111m}In, ¹⁷⁷Lu, ²¹²Bi, ²¹³Bi, ²¹¹At, ⁶²Cu, ⁶⁴Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb ²²³Ra, ²²⁵Ac ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir ¹⁹⁸Au, ¹⁹⁹Au, ¹⁹⁹Au, and ²¹¹Pb, and/or the diagnostic radionuclide is selected from the group consisting of ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ^{99m}Tc, 99Mo, ¹⁰⁵Pd, ¹⁰⁵Rh, ¹¹¹Ag, ¹¹¹ln, ¹²³l, ¹²⁴l, ¹²⁵l, ¹³¹l, ¹⁴²Pr, ¹⁴³Pr, ¹⁴⁹Pm, ¹⁵³Sm, ^{154″1581}Gd, ¹⁶¹Tb, ¹⁶⁶Dy ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. In some embodiments, the first anti-CLDN18.2 antibody and the second anti-CLDN18.2 antibody are the same or different. In some embodiments, both the first and the second anti-CLDN18.2 antibodies comprises a heavy chain variable region comprising a HCDR1 comprising the sequence of SEQ ID NO: 1, a HCDR2 comprising the sequence of SEQ ID NO: 19, and a HCDR3 comprising the sequence of SEQ ID NO: 21, and the light chain variable region comprises a LCDR1 comprising the sequence of SEQ ID NO: 14, a LCDR2 comprising the sequence of SEQ ID NO: 16, and a LCDR3 comprising the sequence of SEQ ID NO: 18.

In some embodiments, the kit is for use in the methods provided herein.

In some embodiments, the present disclosure provides kits comprising the antibody or antigen-binding fragment thereof provided herein and a second therapeutic agent. The kits may be useful in treatment, prevention, and/or amelioration of CLDN18.2 related disease.

In another aspect, the present disclosure provides use of the anti-CLDN18.2 antibody conjugate provided herein or the composition provided herein in the manufacture of a medicament or a diagnostic product for use in the method provided herein.

In certain embodiments, the kit may further comprise a device capable of delivering the kit components through some other route, for example, a syringe for applications such as parenteral delivery, is a syringe for injection of the composition into the body of a subject. In certain embodiments, the anti-CLDN18.2 antibody conjugate provided herein may be provided in the form of a prefilled syringe or autoinjection pen containing a sterile, liquid formulation or lyophilized preparation of antibody (e.g., Kivitz et al., Clin. Ther. 2006, 28:1619-29).

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers. Another component that can be included is instructions for use of the kit.

### EXAMPLES

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1 Introduction

A series of studies have shown that CLDN18.2 as a new target for targeted therapy of gastric cancer has broad application prospects. With precise treatment as the goal, how to maximize the potential beneficiaries, and how to quickly and comprehensively evaluate the molecular pathological characteristics of CLDN18.2 positive gastric cancer patients are urgent problems to be solved.

To solve the problem means to put forward higher requirements for the detection technology of CLDN18.2. However, there are still many limitations in the detection of CLDN18.2.

(1) Single detection method. At present, the commercial antibody is only used for tissue paraffin section, frozen tissue section immunohistochemistry (IHC) detection. With poor discrimination and low sensitivity, they could not distinguish CLDN18.2 or CLDN18.1 (another splice variant of Claudin18), as the amino acid sequences of CLDN18.2 and CLDN18.1 are highly homologous (91% of the amino acid sequences are the same).

(2) The detection method is invasive. The single detection method leads to the CLDN18.2 detection samples are only limited to tissue sections, and the sample collection is invasive and cumbersome, which has a higher requirement for patients.

(3) Heterogeneity of detection specimens: Heterogeneity of advanced gastric cancer is significant. Our found that there may be heterogeneity in tissue expression space and intensity, and heterogeneity in tissue and liquid biopsy specimens of CLDN18.2. The routine pathological molecular diagnosis specimens of patients with advanced stage are mainly endoscopic biopsy specimens, with less tissue content and limited sampling sites; therefore, a single endoscopic tissue specimen test may miss some potential beneficiaries due to the heterogeneity of CLDN18.2 expression.

(4) Static test results: Studies have shown that there are significant differences in the intensity and spatial distribution of target expression, immune microenvironment status of tumor tissue before and after drug treatment, which affect the efficacy of follow-up treatment. Due to the limitation of sampling and detection methods, it is difficult for CLDN18.2 to support the exploration of dynamic monitoring. Therefore, for these problems, it is the best solution to explore and integrate CLDN18.2 noninvasive, comprehensive, real-time dynamic detection technology, to achieve a comprehensive evaluation of all lesions, and to summarize the characteristics of potential beneficiaries combined with curative effect, and to guide diagnosis and treatment.

Therefore, it is of great significance to develop molecular probes with high specificity for the diagnosis of CLDN18.2 and the evaluation of subsequent immune checkpoint antibody response by using the comprehensive, non-invasive, real-time and dynamic characteristics of modern molecular imaging technology. Although there are many nuclide molecular probes focusing on HER2 positive gastric cancer, and the technology is relatively mature, the reports of CLDN18.2 nuclide probe at home and abroad are still blank. The invention took CLDN18.2 targeted monoclonal antibody 18B10 as an example, used the next generation nuclide ¹²⁴I/⁶⁴Cu/⁸⁹Zr to label 18B10 antibody, and in vivo study showed that it had significantly high uptake in CLDN18.2 high expression tumor, suggesting that the nuclide labeled 18B10 antibody, as a specific probe targeting CLDN18.2, is a potential imaging agent for tumor immunotherapy screening, treatment process monitoring and efficacy evaluation.

The present invention provides, at least in part, a next generation nuclide ¹²⁴I/⁶⁴Cu/⁸⁹Zr to labeled CLDN18.2 targeted monoclonal antibody (taking 18B10 as an example) and its preparation method and application.

The conception of the invention is as follows: ¹²⁴I (T_{1/2}=4.2 d), ⁶⁴Cu (T_{1/2}=12.7 h), ⁸⁹Zr (T_{1/2}=78.4 h) are new solid target PET nuclides, which have broad clinical application prospects. Compared with ¹⁸F (T_{1/2}=110 min) and ¹¹C (T_{1/2}=20.4 min), ¹²⁴I/⁶⁴Cu/⁸⁹Zr has a relatively long half-life, which is conducive to long-term imaging research and transportation of radionuclides, and the liver uptake is low, which is conducive to the detection of liver lesions. Monoclonal antibodies, which bind to only one epitope (antigenic determinant) on the antigen molecule, can be used as a molecular probe to study the structure and function of antigen and elucidate the mechanism. It is easy to determine the location and the distribution of biomacromolecules (proteins, nucleic acids, enzymes, etc.) in vivo using the nuclide labeled monoclonal antibody.

In order to achieve the purpose of the invention, in the first aspect, the invention provides a next generation nuclide labeled CLDN18.2 targeted monoclonal antibody, which is a radionuclide ¹²⁴I/⁶⁴Cu/⁸⁹Zr labeled CLDN18.2 targeted monoclonal antibody.

In the second aspect, the invention provides a preparation method of ¹²⁴I/⁶⁴Cu/⁸⁹Zr-CLDN18.2 targeted monoclonal antibody (taking 18B10 as an example). By NBS method, ¹²⁴I was labeled to the ortho para position of phenyl hydroxyl group in the molecular structure of monoclonal antibody, the method includes: 0.5-1.5 mL 0.1M pH7.2 PB buffer, 0.5-10 mg 18B10 and 6-120 μg N-bromosuccinimide were added to 0.5-1.0 mL 25-90 KBq/μL Na¹²⁴I solution, and cultured at 37° C. for 60 s, then 0.05-0.2 mL 10% human serum albumin was added to terminate the reaction; the final reaction solution was purified by PD-10 column to obtain the target product.

⁶⁴Cu/⁸⁹Zr labeled CLDN18.2 targeted monoclonal antibody was achieved by using bifunctional chelator (taking 18B10 as an example): DFO equivalent to 6 moles of monoclonal antibody was added to 18B10 solution with concentration of 2-5 mg/mL, and the pH value of the reaction system was adjusted to 8.5 with deionized 0.1 M sodium bicarbonate solution; DFO-18B10 was obtained by reaction at 4-37° C. for 4-24 h. 15 μg-2 mg of labeled precursor was added into 0.1-1.0 mL of 0.1 M sodium acetate solution with pH 5.5, then 25-100 MBq fresh ⁶⁴Cu/⁸⁹Zr solution was added and the pH value was adjust to 7.0; the temperature was controlled at 40° C. for 30 min; the final reaction solution was purified by PD-10 column to obtain the target product.

The radiochemical purity of the target product was 98.76% when the labeling rate was more than 95% and purified by PD-10 column.

Before use, the PD-10 column was first equilibrated with 0.01 M pH 7.4 PBS solution, 5 mL PBS was added each time, the column was dried at the gravity flow rate, repeating 5 times; then the target product was purified with 0.01 M pH 7.4 PBS solution.

In vitro stability analysis of ¹²⁴I-18B10 showed that it could maintain good stability in 0.01 M, pH 7.4 PBS solution at 25° C. for 48 h, and the radiochemical purity was 97.89±0.05%.

In the third aspect, the invention provides the application of ¹²⁴I/⁶⁴Cu/⁸⁹Zr-CLDN18.2 targeted monoclonal antibody (taking 18B10 as an example) in the preparation of tumor imaging agent and therapeutic drug targeting CLDN18.2.

In the fourth aspect, the invention provides the application of ¹²⁴I/⁶⁴Cu/⁸⁹Zr-CLDN18.2 targeted monoclonal antibody (taking 18B10 as an example) in the preparation of PET/CT molecular imaging probe.

In the fifth aspect, the invention provides a tumor imaging agent targeting CLDN18.2, which comprises the nuclide labeled CLDN18.2 targeted monoclonal antibody.

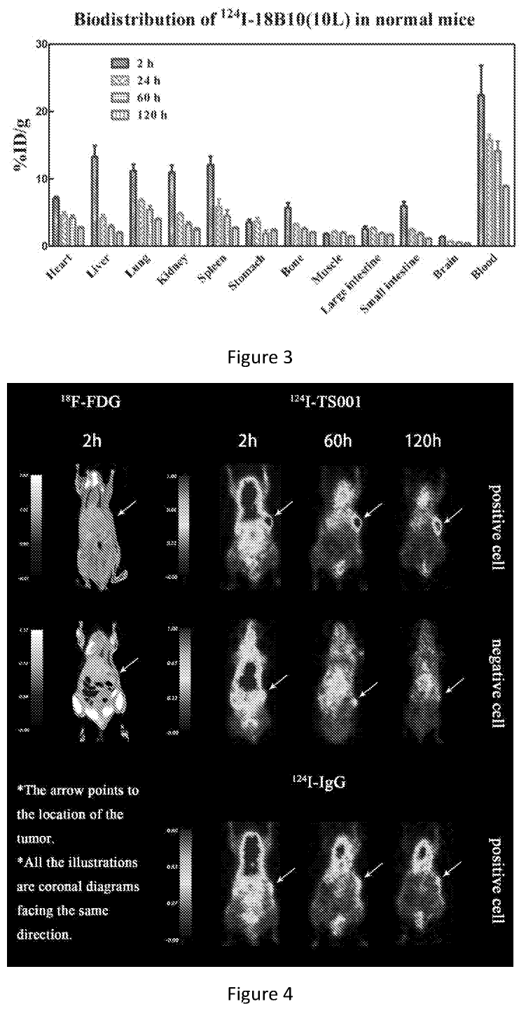

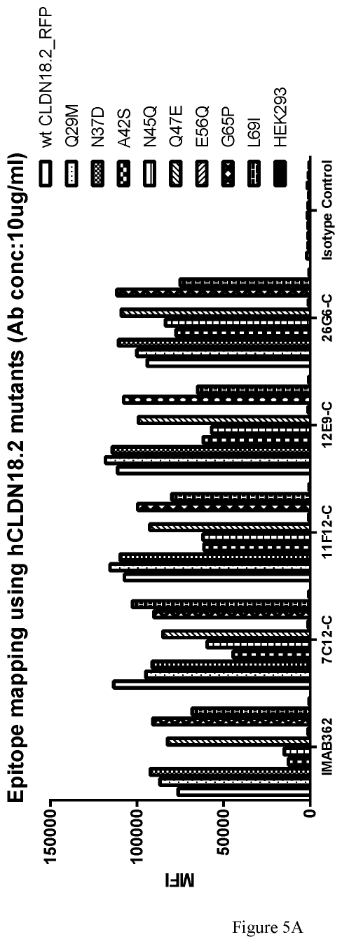

A new generation of nuclide labeled monoclonal antibody (¹²⁴I-18B10) can specifically bind to CLDN18.2 on the surface of tumor cells; there was no specific distribution of organs and tissues in normal mice except blood; in the PET imaging study of PDX bearing mice, ¹⁸F-FDG uptake was very low in both CLDN18.2 positive and CLDN18.2 negative tumor bearing models, as shown in FIG. 4. The distribution of ¹²⁴I-18B10 in CLDN18.2 positive PDX bearing mice changed dynamically with time: the uptake signal in tumor sites significantly increased at 60 h comparing to 2 h; at 120 h, the uptake signal in cardiac blood pool disappeared, and only the signal in the tumor could be observed. There was no specific accumulation of ¹²⁴I-18B10 in tumor sites in CLDN18.2 negative PDX bearing mice and of ¹²⁴I-IgG in CLDN18.2 positive PDX bearing mice. These results indicate that ¹²⁴I-18B10 probe is specifically accumulated in CLDN18.2 positive tumors, and has the characteristics of high sensitivity and specificity. It can be used as an imaging agent to noninvasively detect the expression of CLDN18.2 in patients' tumors in the future.

In summary, the invention has at least the following advantages and beneficial effects: the novel PET molecular probe ¹²⁴I/⁶⁴Cu/⁸⁹Zr-CLDN18.2 targeted monoclonal antibody provided by the invention has stable property, good imaging effect, high affinity and functional activity to CLDN18.2, and is expected to be used to monitor the expression of CLDN18.2 in systemic lesions in real time and noninvasively, monitor the heterogeneity of CLDN18.2 expression in the same lesion and different lesions, and observe the change of CLDN18.2 expression during treatment, so as to screen patients, monitor curative effect, early warn drug resistance and/or recurrence and metastasis, and realize the individualized and precise treatment of targeted tumor drugs.

In the invention, the next generation PET nuclide ¹²⁴I/⁶⁴Cu/⁸⁹Zr is used, which can realize the labeling of monoclonal antibody and has little influence on the activity of monoclonal antibody; in addition, the liver non-specific uptake is low, which is conducive to the detection of liver lesions; moreover, the background of the whole body is low, which has a good tumor to non-tumor ratio and is conducive to the observation of tumor lesions.

### Example 2 Preparation of a Next Generation Nuclide Labeled Monoclonal Antibody Targeting CLDN18.2 (Taking ¹²⁴I-18B10 as an Example)

The CLDN18.2 targeted monoclonal antibody 18B10 used in the study was provided by MabSpace Biosciences (Suzhou) Co., Ltd, which is cross-reactive with both human and mouse CLDN18.2 and can be tested in mice for preclinical research. PD-10 pre-loaded gel column was purchased from GE (US.).

¹²⁴I-18B10 was prepared by NBS reaction: 0.8 mL 0.1M pH7.2 PB buffer, 0.1 mL (29.2 mg/mL) 18B10 monoclonal antibody solution (prepared with H₂O) and 36 μg NBS were added to 1.0 mL 59.2 KBq/μL Na¹²⁴I solution in turn, and cultured at 37° C. for 60 s, then 0.1 mL 10% human serum albumin was added to terminate the reaction; the final reaction solution was purified by PD-10 column to obtain the target product.

Before use, the PD-10 column was first equilibrated with 0.01 M pH 7.4 PBS solution, 5 mL PBS was added each time, the column was dried at the gravity flow rate, repeating 5 times; then the target product was purified with 0.01 M pH 7.4 PBS solution.

Radio-TLC and Radio-HPLC were used to determine the radiolabeling rate and radiochemical purity. Radio-TLC detection: 2 μL free Na¹²⁴I with radioactivity of 37-74 kBq (1-2 μCi) and purified ¹²⁴I-18B10 were added to 20 μL of saturated EDTA. 2 μL above solution was dropped 1 cm from the bottom of Xinhua No. 1 filter paper and placed in the normal saline development system. After complete development, the filter paper was taken out and dried for Radio-TLC detection. Radio-HPLC detection: 2 μL free Na¹²⁴I with radioactivity of 37-74 kBq (1-2 ρCi) and purified ¹²⁴I-18B10 were diluted into 50 μL 0.01M pH 7.4 PBS for Radio-HPLC analysis. Analysis conditions: Agilent Bio SEC-3 gel filtration/volume exclusion chromatography column, flow rate 1 mL/min, mobile phase 0.01 M pH 7.4 PBS.

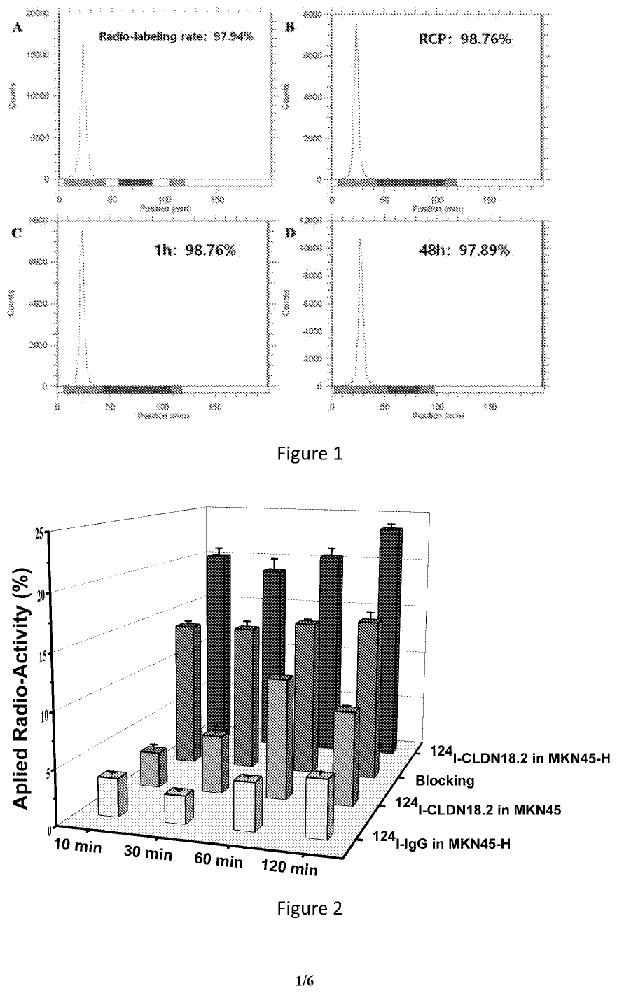

The radiolabeling rate and radiochemical purity of the target product ¹²⁴I-18B10 were 97.94% and 98.76%, respectively. The radiochemical purity of the target product ¹²⁴I-18B10 remained about 98% after 48 h in PBS (FIG. 1).

### Example 3 Uptake of a Next Generation Nuclide Labeled Monoclonal Antibody Targeting CLDN18.2 (Taking ¹²⁴I-18B10 as an Example)

Through transfection of CLDN18.2 into MKN45 cells and screening, MKN45-CLDN18.2 high cell line with high expression of CLDN18.2 was obtained. MKN45 cells were used as negative control cells. The seeded cells were evenly cultured in 24 well plates overnight to make them fully adhere to the wall. On the second day, 1 mCi ¹²⁴I-18B10 or ¹²⁴I-hIgG was added and cultured for 10 min, 30 min, 60 min and 120 min, respectively. In blocking group, 0.5 mg/well cold precursor 18B10 was added to block the uptake signal. After culture, the cells were washed with PBS for three times, and the lysate was collected. The uptake signal was detected and analyzed with γ counter.

The results (FIG. 2) showed that MKN45-CLDN18.2 high cells showed a high uptake rate of ¹²⁴I-18B10 at 10 min, which increased with time. When cold precursor was added for blocking, the uptake rate decreased significantly, indicating that the uptake signal was CLDN18.2-specific. Both the uptake of ¹²⁴I-hIgG by MKN45-CLDN18.2 high cells and ¹²⁴I-18B10 by MKN45 cells were low.

### Example 4 Biodistribution of a Next Generation Nuclide Labeled Monoclonal Antibody Targeting CLDN18.2 (Taking ¹²⁴I-18B10 as an Example) in Normal Mice

¹²⁴I-18B10 (0.74 MBq, 200 μL) was injected into normal Kunming mice via tail vein. The heart, liver, lung, kidney, spleen, stomach, bone, muscle, large intestine, small intestine, brain, blood and other organs and tissues of mice were isolated at 2 h, 24 h, 60 h and 120 h after injection. After grinding, the uptake signal was detected and analyzed by γ counter, and the ID %/g of ¹²⁴I-18B10 probe was obtained.

In normal mice (FIG. 3), blood uptake was the highest at all time points. In addition to blood, the uptake in liver, kidney, lung and spleen at 2 hours was higher than that in other organs and tissues. The uptake of all organs decreased with time. Compared with blood, the uptake of other organs tends to be lower.

### Example 5 PET Imaging of a Next Generation Nuclide Labeled Monoclonal Antibody Targeting CLDN18.2 (Taking ¹²⁴I-18B10 as an Example) in PDX Tumor Bearing Mice

By immunohistochemical (IHC) method, CLDN18.2 positive PDX (positive intensity of 3+) and CLDN18.2 negative PDX were selected for inoculation. All tumor tissues were subcutaneously injected into the right lower limb of nude mice. When the diameter of PDX tumor was 0.8 cm, the tumor bearing mice were divided into groups. The CLDN18.2 positive PDX bearing mice with were divided into three groups: ¹⁸F-FDG group, ¹²⁴I-18B10 group and ¹²⁴I-hIgG group. These three groups were injected via tail vein with ¹⁸F-FDG, 18.5 MBq ¹²⁴I-18B10 and 18.5 MBq ¹²⁴I-hIgG respectively. CLDN18.2 negative PDX tumor bearing mice were also divided into the same three groups. At 2 h, 60 h and 120 h after injection, the animals were anesthetized with isoflurane (2% isoflurane-30% oxygen/air), and the imaging was performed on Micro PET/CT.

¹⁸F-FDG uptake was very low in both CLDN18.2 positive and CLDN18.2 negative tumor bearing models, as shown in FIG. 4. The distribution of ¹²⁴I-18B10 in CLDN18.2 positive PDX bearing mice changed dynamically with time: at 2 h, the uptake signals were detected in cardiac blood pool and CLDN18.2 positive tumor sites; at 60 h, the uptake signal in cardiac blood pool was significantly reduced, while it increased in the tumor; at 120 h, the uptake signal in cardiac blood pool disappeared, and only the signal in the tumor could be observed. The uptake signals of ¹²⁴I-18B10 in CLDN18.2 negative PDX bearing mice in all tissues and tumors were quite similar at different time points, indicating there was no specific accumulation in CLDN18.2 negative tumor. As a negative control, the uptake signal of ¹²⁴I-IgG in CLDN18.2 positive PDX bearing mice was found high in cardiac blood pool rather than in the tumor at each time points. These results indicate that ¹²⁴I-18B10 probe is specifically accumulated in CLDN18.2 positive tumors, and there was still significant signal after long time (120 h) imaging. It has the characteristics of high sensitivity and specificity, and can be used as an imaging agent to noninvasively detect the expression of CLDN18.2 in patients' tumors in the future.

The next generation nuclide ¹²⁴I/⁶⁴Cu/⁸⁹Zr labeled CLDN18.2 targeted monoclonal antibody provided by the invention has stable properties, good imaging effect, high affinity and functional activity for CLDN18.2, and is conducive to the diagnosis and accurate staging of CLDN18.2 positive tumor lesions. It is confirmed by preclinical animal studies that it has the potential to become a targeted CLDN18.2 imaging agent with good application prospects.

### Example 6: Epitope Mapping of Selected Antibodies Via Site-Directed Mutagenesis on the CLDN18.2 Amino Acids Different with CLDN18.1

1. Generation of Human CLDN18.2-mRFP and Human CLDN18.1-mRFP Constructs

The cDNA coding for human CLDN18.1 (amino acid 1-261, SEQ ID NO: 31)-mRFP1 (amino acid 1-225) and human CLDN18.2 (amino acid 1-261, SEQ ID NO: 30)-mRFP1 (amino acid 1-225) were synthesized in vitro (SEQ ID NO: 52 and SEQ ID NO: 53 are the amino acid sequences, respectively). The PCR product was then cloned into the pcDNA3.1 (+) vector by method of homologous recombination using Syno assembly mix reagent (Synbio) following manufacturer's instructions. Plasmid was purified by using QIAGEN Plasmid Mega Kit (QIAGEN).

According to the sequence of human CLDN18.1 and CLDN18.2 (Genbank accession number: splice variant 1 (CLDN18.1): NP_057453, NM_016369, and splice variant 2 (CLDN18.2): NM_001002026, NP_001002026), 8 different amino acids are located between 28-70, which may be the determinant of the specific binding to human CLDN18.2 not to CLDN18.1. Using wild-type human CLDN18.2-mRFP plasmid generated above as template, two segments of an integrated sequence were generated with the primers. The variants of human CLDN18.2-mRFP with single amino acid changed into that of human CLDN18.1 at the designated position were amplified by overlapping PCR using the primers. The specific mutations are on Q29M, N37D, A42S, N45Q, Q47E, E56Q, G65P and L69I. Variants of human CLDN18.1-mRFP with single amino acid changed at designated position were amplified by overlapping PCR using primers. The specific mutations are on M29Q, D37N, S42A, Q45N, E47Q, Q56E, P65G, I69L. The PCR product was then cloned into the pcDNA3.1 (+) vector by method of homologous recombination. The human CLDN18.2-mRFP variants were identified and confirmed by sequencing the individual positive clones.

Subsequently, these plasmids of mutants and wild-type human CLDN18.2-mRFP or human CLDN18.1-mRFP were transfected into HEK293 cell line. First, 5×10⁶ HEK293 cells were seeded into 60 mm dish at a ratio of 60%˜80% for transfection. 10 g DNA in 400 μl×HBS and 10 μl 125 kDa linear PEI transfection reagent (dissolved in 1×HBS, 1 mg/ml stock solution) was mixed to reach a DNA/PEI ratio of 1:2.5. Next the mixture was added into HEK293 cell culture drop by drop. After 6-8 hours, the transfected cells were replaced with complete DMEM for overnight. At 24 hours after transfection, cells were collected for FACS analysis using chimeric antibodies.

Amino acid sequence of human
CLDN18.1 (SEQ ID NO: 31)
MSTTTCQVVAFLLSILGLAGCIAATGMDMWSTQDLYDNPVTSVFQ
YEGLWRSCVRQSSGFTECRPYFTILGLPAMLQAVRALMIVGIVLG
AIGLLVSIFALKCIRIGSMEDSAKANMTLTSGIMFIVSGLCAIAG
VSVFANMLVTNFWMSTANMYTGMGGMVQTVQTRYTFGAALFVGWV
AGGLTLIGGVMMCIACRGLAPEETNYKAVSYHASGHSVAYKPGGF
KASTGFGSNTKNKKIYDGGARTEDEVQSYPSKHDYV
Amino acid sequence of human
CLDN18.1-mRFP1 (SEQ ID NO: 52):
MSTTTCQVVAFLLSILGLAGCIAATGMDMWSTQDLYDNPVTSVFQ
YEGLWRSCVRQSSGFTECRPYFTILGLPAMLQAVRALMIVGIVLG
AIGLLVSIFALKCIRIGSMEDSAKANMTLTSGIMFIVSGLCAIAG
VSVFANMLVTNFWMSTANMYTGMGGMVQTVQTRYTFGAALFVGWV
AGGLTLIGGVMMCIACRGLAPEETNYKAVSYHASGHSVAYKPGGF
KASTGFGSNTKNKKIYDGGARTEDEVQSYPSKHDYVMASSEDVIK
EFMRFKVRMEGSVNGHEFEIEGEGEGRPYEGTQTAKLKVTKGGPL
PFAWDILSPQFQYGSKAYVKHPADIPDYLKLSFPEGFKWERVMNF
EDGGVVTVTQDSSLQDGEFIYKVKLRGTNFPSDGPVMQKKTMGWE
ASTERMYPEDGALKGEIKMRLKLKDGGHYDAEVKTTYMAKKPVQL
PGAYKTDIKLDITSHNEDYTIVEQYERAEGRHSTGA
Amino acid sequence of human
CLDN18.2 (SEQ ID NO: 30)
MAVTACQGLGFVVSLIGIAGIIAATCMDQWSTQDLYNNPVTAVFN
YQGLWRSCVRESSGFTECRGYFTLLGLPAMLQAVRALMIVGIVLG
AIGLLVSIFALKCIRIGSMEDSAKANMTLTSGIMFIVSGLCAIAG
VSVFANMLVTNFWMSTANMYTGMGGMVQTVQTRYTFGAALFVGWV
AGGLTLIGGVMMCIACRGLAPEETNYKAVSYHASGHSVAYKPGGF
KASTGFGSNTKNKKIYDGGARTEDEVQSYPSKHDYV
Amino acid sequence of human
CLDN18.2-mRFP1 (SEQ ID NO: 53):
MAVTACQGLGFVVSLIGIAGIIAATCMDQWSTQDLYNNPVTAVFN
YQGLWRSCVRESSGFTECRGYFTLLGLPAMLQAVRALMIVGIVLG
AIGLLVSIFALKCIRIGSMEDSAKANMTLTSGIMFIVSGLCAIAG
VSVFANMLVTNFWMSTANMYTGMGGMVQTVQTRYTFGAALFVGWV
AGGLTLIGGVMMCIACRGLAPEETNYKAVSYHASGHSVAYKPGGF
KASTGFGSNTKNKKIYDGGARTEDEVQSYPSKHDYVMASSEDVIK
EFMRFKVRMEGSVNGHEFEIEGEGEGRPYEGTQTAKLKVTKGGPL
PFAWDILSPQFQYGSKAYVKHPADIPDYLKLSFPEGFKWERVMNF
EDGGVVTVTQDSSLQDGEFIYKVKLRGTNFPSDGPVMQKKTMGWE
ASTERMYPEDGALKGEIKMRLKLKDGGHYDAEVKTTYMAKKPVQL
PGAYKTDIKLDITSHNEDYTIVEQYERAEGRHSTGA

2. Binding of CLDN18.2 Chimeric Antibodies to Site-Mutated HEK293-CLDN18.2 or HEK293-CLDN18.1 Cell

The transfected HEK293-CLDN18.2 or HEK293-CLDN18.1 cell were resuspended in PBS with 2% BSA at density of 10\hat{ }5/well, 100 μl/well. Cells were washed 3 times by FACS washing buffer (PBS+2% FBS) and incubated with 100 μl/well 10 μg/ml chimeric antibodies and IMAB362 each well at 4° C. for 30 min. Next, cells were washed 3 times by FACS washing buffer and incubated with 100 μl/well goat anti-hIgG(H+L)-FITC (1:200 dilution) at 4° C. for another 30 min. Finally, cells were washed 3 times by FACS washing buffer and analyzed by Flow Cytometry. To analyze binding to CLDN18.2 transfected cells, the RFP positive cells were used for control gating.

IMAB362 was expressed it according to the sequence disclosed in US2009169547A1. The single cell clone with a highest signal was selected and amplified for cell banking.

Heavy chain variable region of IMAB362
(SEQ ID NO: 72)
QVQLQQPGAELVRPGASVKLSCKASGYTFTSYWINWVKQRPGQGL
EWIGNIYPSDSYTNYNQKFKDKATLTVDKSSSTAYMQLSSPTSED
SAVYYCTRSWRGNSFDYWGQGTTLTVSS
Light chain variable region of IMAB362
(SEQ ID NO: 73)
DIVMTQSPSSLTVTAGEKVTMSCKSSQSLLNSGNQKNYLTWYQQK
PGQPPKLLIYWASTRESGVPDRFTGSGSGTDFTLTISSVQAEDL
AVYYCQNDYSYPFTFGSGTKLEIK

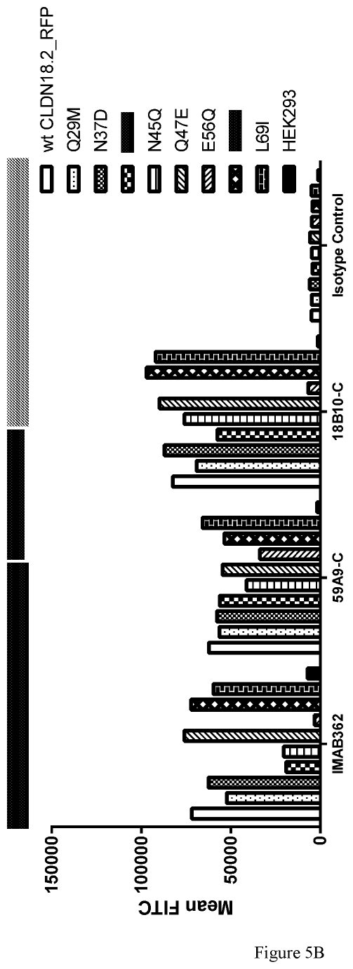

The percentage of binding signal of these chimeric antibodies to mutated CLDN18.2 variants relative to that the wild-type was calculated and summarized in Table 4. As shown in FIG. 5A-5B, binding of 18B10-C (i.e. chimeric antibody of 18B10 fused to human IgG1 constant region) was completely lost when E56 was mutated to Q. This change also applied to IMAB362 and other chimeric antibodies, except for 59A9-C. In addition, we found that other amino acids, such as A42, N45, also contributed to binding of IMAB362 and other antibodies at some extent but not so for 18B10-C.

TABLE 4
The binding percentage of mutated human CLDN18.2
as compared with wild-type CLDN18.2 (%)

| Mutations on | | | | | | | |
|---|---|---|---|---|---|---|---|
| CLDN18.2 | IMAB362 | 7C12-C | 11F12-C | 12E9-C | 26G6-C | 59A9-C | 18B10-C |
| Wild-type | 100.00 | 100.00 | 100.00 | 100.00 | 100.00 | 100.00 | 100.00 |
| Q29M | 113.89 | 83.52 | 107.78 | 106.33 | 106.53 | 90.37 | 83.50 |
| N37D | 121.11 | 80.20 | 102.19 | 102.71 | 117.72 | 92.66 | 105.63 |
| A42S | 15.76 | 38.69 | 56.86 | 55.10 | 82.23 | 90.02 | 69.23 |
| N45Q | 18.87 | 52.10 | 57.38 | 50.77 | 88.69 | 65.04 | 91.97 |
| Q47E | 108.17 | 74.74 | 86.13 | 89.07 | 116.12 | 87.47 | 109.00 |
| E56Q | 0.83 | 0.48 | 0.27 | 0.35 | 0.34 | 52.74 | 6.57 |
| G65P | 119.26 | 79.44 | 92.67 | 96.73 | 118.87 | 85.55 | 118.11 |
| L69I | 89.27 | 90.38 | 74.28 | 58.10 | 79.74 | 105.74 | 111.70 |

### Example 7: Generation and Characterization of Humanized Antibodies

### 1. Generation, Expression and Purification of Humanized Antibodies

### 18B10

Human germline framework sequence VK/4-1 for light chain and VH/1-46 for heavy chain were used for CDR grafting, respectively.

Heavy chain (HC) variants 1, 2 and 3 were obtained by direct grafting the three CDRs to the germline sequence (18B10 HC germline, SEQ ID NO: 23) and back mutation of R71I, T73K for HC variant 1 (Hul8B 10_Ha, SEQ TD NO: 25), back mutation of R71I, T73K, T28 S, M69L for HC variant 2 (Hul8B 10_Hb, SEQ ID NO: 27) and back mutation of R71I, T73K, T28 S, M69L, R38K, M48I for HC variant 3 (Hul8B10_Hc, SEQ TD NO: 29), respectively.

(1) Germline sequence for 18B 10 HC:

VH/1-46
(18B10-germline, SEQ ID NO: 23):
QVQLVQSGAEVKKPGASVKVSCKASGYTFTSYYMHWVRQAPGQGL
EWMGIINPSGGSTSYAQKFQGRVTMTRDTSTSTVYMELSSLRSED
TAVYYCAR
VH/1-46 variant 1
(Hu18B10_Ha, SEQ ID NO: 25):
QVQLVQSGAEVKKPGASVKVSCKASGYTFTGYNMNWVRQAPGQGL
EWMGNIDPYYGGTSYNQKFKGRVTMTIDKSTSTVYMELSSLRSED
TAVYYCARMYHGNAFDYWGQGTTVTVSS
VH/1-46 variant 2
(Hu18B10_Hb, SEQ ID NO: 27):
QVQLVQSGAEVKKPGASVKVSCKASGYSFTGYNMNWVRQAPGQGL
EWMGNIDPYYGGTSYNQKFKGRVTLTIDKSTSTVYMELSSLRSED
TAVYYCARMYHGNAFDYWGQGTTVTVSS
VH/1-46 variant 3
(Hu18B10_Hc, SEQ ID NO: 29):
QVQLVQSGAEVKKPGASVKVSCKASGYSFTGYNMNWVKQAPGQGL
EWIGNIDPYYGGTSYNQKFKGRVTLTIDKSTSTVYMELSSLRSED
TAVYYCARMYHGNAFDYWGQGTTVTVSS

Light chain (LC) variant 1 and 2 were obtained by direct grafting the three CDRs to germline sequence (18B10 LC germline, SEQ ID NO: 24) and no back mutation for variant 1 (Hul8B10_La, SEQ ID NO: 26) and S63T, I21M for LC variant 2 (Hul8B10_Lb, SEQ ID NO: 28), respectively.

### (2) Germline Sequence for 18B10 LC:

VK/4-1
(18B10 LC germline, SEQ ID NO: 24)
DIVMTQSPDSLAVSLGERATINCKSSQNNKNYLAWYQQKPGQPPK
LLIYWASTRESGVPDRFSGSGSGTDFTLTISSLQAEDVAVYYCQQ
YYSTP
VK/4-1 variant 1
(Hu18B10_La, SEQ ID NO: 26)
DIVMTQSPDSLAVSLGERATINCKSSQSLLNSGNLKNYLTWYQQK
PGQPPKLLIYWASTRKSGVPDRFSGSGSGTDFTLTISSLQAEDVA
VYYCQNDYSYPLTFGGGTKVEIK
VK/4-1 variant 2
(Hu18B10_Lb, SEQ ID NO: 28)
DIVMTQSPDSLAVSLGERATMNCKSSQSLLNSGNLKNYLTWYQQK
PGQPPKLLIYWASTRKSGVPDRFTGSGSGTDFTLTISSLQAEDVA
VYYCQNDYSYPLTFGGGTKVEIK

The combination of the above heavy chain variable regions and light chain variable regions generate the following humanized 18B10 antibodies: 18B10-HaLa (having a VH of SEQ TD NO: 25 and a VL of SEQ ID NO: 26), 18B10-HbLa (having a VH of SEQ ID NO: 27 and a VL of SEQ ID NO: 26), 18B10-HcLa (having a VH of SEQ TD NO: 29 and a VL of SEQ TD NO: 26), 18B10-HaLb (having a VH of SEQ TD NO: 25 and a VL of SEQ TD NO: 28), 18B10-HbLb (having a VH of SEQ TD NO: 27 and a VL of SEQ ID NO: 28), 18B10-HcLb (having a VH of SEQ TD NO: 29 and a VL of SEQ TD NO: 28).

The humanized variants of the heavy chain and light chain of 18B10 are linked to human IgG1 heavy chain constant region and kappa light chain constant region as shown below:

Human IgG1 heavy chain constant
region (SEQ ID NO: 49):
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
Human Kappa light chain constant
region (SEQ ID NO: 50):
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC

The variable regions of the above heavy chain and light chain cDNAs were synthesized and fused with the constant region of human IgG1 and human kappa. The heavy chain and light chain of the selected antibody genes were cloned into an expression vector and the large-scale DNA was prepared using Plasmid Maxiprep System from Qiagen. Transfection was carried out using the ExpiFectamine™ CHO Reagent from Invitrogen according to the manufacturer's protocol. Supernatants were harvested when the cell viability was around 60%. The cell culture supernatant was filtered through 0.22 um filtration capsule to remove the cell debris. Load the supernatant onto a pre-equilibrated Protein-A affinity column. Then Protein A resin inside the column was washed with equilibration buffer (PBS), and 25 mM citrate (pH3.5) was used to elute the antibody. The pH was adjusted to about 6.0-7.0 with 1M Tris-base (pH 9.0). The endotoxin was controlled below 1 EU/mg. The purified antibody was then characterized by SDS-PAGE and SEC-HPLC.

### 2. Binding to Human and Mouse CLDN18.2

Log-phase HEK293-CLDN18.2 and NUGC4 cells were re-suspended in PBS. After 3× cell wash by using FACS washing buffer (PBS+2% FBS), 100 l/well diluted humanized antibodies with a range from 400 nM to 0.002 nM were added into each well for incubation at 4° C. for 30 min. Again, cells were washed 3 times by using FACS washing buffer and then incubated with 100 l/well goat anti-mIgG-FITC (1:400 dilution) at 4° C. for another 30 min. After a final 3× wash using FACS washing buffer, cells were analyzed by flow Cytometry.

All humanized antibody variants were tested head to head with the chimeric one in order to screen the best. All the variants retained their binding completely. Next, 18B10-HaLa, which had only one back mutation, was tested for its binding to HEK293-mouse CLDN18.2 cells. 18B10-HaLa could bind well to mouse CLDN18.2 with a better potency and a higher MFI than IMAB362, indicating that 18B10-HaLa had a good cross-reactivity to mouse.

### 3. Affinity Analysis of Humanized CLDN18.2 Antibodies by KinExA

18B10-HaLa and IMAB362 were evaluated head to head by KinExA for their affinity binding to CLDN18.2 expressing cells. Following KinExA 4000 (Sapidyne Instruments Inc.)'s instruction, 200 mg PMMA hard beads (Sapidyne, #440176) were coated with 30 g Goat anti-human IgG Fc antibody for 2 h, and then blocked by 10 mg/ml BSA for 1 h. Two gastric cell lines, NUGC4 and KATOIII (ATCC, Cat #HTB-103), were collected at log-phase and mixed with 0.2 nM 18B10-HaLa or IMAB362. The cell-antibody mixture was 2-fold diluted using 0.2 nM 18B10-HaLa or IMAB362 and incubated at room temperature for 3 h. Amount of the free antibodies was increasing along with dilution. These free antibodies were captured by Goat anti-human IgG Fc coated beads, and subsequently labeled by 1 μg/ml Alexa Fluor 647-anti-human IgG for readout.

The binding affinity of each antibody was summarized in Table 5. Kd of 18B10-HaLa binding to NUGC4 cell and KATOIII was approximately 0.3 nM, which was over 8-fold higher than that of IMAB362. This was consistent with the above FACS binding results.

TABLE 5
Kd of CLDN18.2 antibodies to gastric cell lines

| Kd (nM) | 18B10-HaLa | IMAB362 |
|---|---|---|
| NUGC4 | 0.303 | 2.58 |
| KATOIII | 0.315 | ND |

### 4. CDC Assay on HEK293-CLDN18.2 Cell

18B10-HaLa was tested head to head with IMAB362 in the CDC activity assay. 18B10-HaLa had more than 20-fold higher CDC activity than IMAB362. The percentage of 18B10-HaLa-dependent specific cell killing reached to 86% at a concentration of 0.3 μg/ml, while IMAB362 had no cell killing at the same concentration.

### 5. Binding and Cytotoxic Effect on MKN45-CLDN18.2 Cell

Cell binding assay was same as above. All humanized variants of 18B10 bound to cells with a comparable affinity with chimeric 18B10. 18B10-HaLa, with only one back mutation, was selected for further ADCC activity study.

ADCC activity was tested using Jurkat-NFAT-luc-FcγRIIIA-V176 cells as effector cells and MKN45-CLDN18.2 cells as target cells. 18B10-HaLa had a much lower EC50 (0.05 μg/ml) than IMAB362, consistent with that of chimeric 18B10.

### 6. Binding and Cytotoxic Effect on NUGC4 Cell

Cell binding and ADCC assays were same as above. 18B10-HaLa showed a much better ADCC potency (EC50˜0.59 μg/ml) as compared to IMAB362.

### 7. ADCC Assay Using NUGC4 as Target Cell and PBMC as Effector Cell

Log-phase NUGC4 cells were resuspended in RPMI1640 with 10% FBS. Cells were pre-seeded into 96-well U bottom plate at 1×10\hat{ }4 cells per well. Anti-CLDN18.2 antibodies and IMAB362 were gradient diluted in RPMI1640 with 10% FBS and added into the plate above at a final concentration from 200 to 0.2 μg/ml and incubated at 37° C. for 30 min. Frozen PBMC from Miao Shun (Shanghai) Biological & Technology Co., Ltd were removed from liquid nitrogen and put into 37° C. water bath immediately. After centrifugation, cells were resuspended in RPMI1640 plus 10% FBS and the seeded into 96-well U bottom plate mentioned above at 40×10\hat{ }4 cells per well. The plate was then placed in the incubator at 37° C. for 5 hours.

After incubation, the plate was equilibrated to 22° C. LDH was detected by using Promega CytoTox-ONE Homogeneous Membrane Integrity Assay Kit (G7892) or the CytoTox 96® Non-Radioactive Cytotoxicity Assay (G1780). After adding the Lysis, Reagent and Stop Solutions following manufacturer's instruction, fluorescence was measured under an excitation wavelength of 560 nm and an emission wavelength of 590 nm (G7892), or the absorbance at 490 nm or 492 nm (G1780).

18B10-HaLa showed a much better ADCC potency than IMAB362. Due to non-fit to regression curve, EC50 may not be calculated accurately.

### 8. Epitope Mapping of the Selected Antibodies Using Site-Directed Mutagenesis on Human CLDN18.2

Using the same method and human CLDN18.2-mRFP plasmid, 42 amino acids between human CLDN18.2 28-80 as listed below were replaced by alanine one at a time. These variants were amplified by overlapping PCR using primers. The specific mutations are Q28A, Q29A, W30A, S31A, T32A, Q33A, D34A, L35A, Y36A, N37A, N38A, V40A, T41A, V43A, F44A, N45A, Y46A, Q47A, L49A, W50A, R51A, S52A, V54A, R55A, E56A, E56A, S57A, S58A, F60A, T61A, E62A, R64A, Y66A, F67A, T68A, L69A, L70A, L72A, M75A, L76A, Q77A, V79A, R80A. The PCR product was then cloned into the pcDNA3.1 (+) vector by method of homologous recombination using Syno assembly mix reagent (Synbio) following manufacturer's instructions. Plasmid was purified by using QIAGEN Plasmid Mega Kit (QIAGEN).

Subsequently, these plasmids of mutants and wild-type CLDN18.2-mRFP were transfected into HEK293 cell. Cells were analyzed by flow cytometry 24 hours after transfection.

Binding of 18B10-HaLa was completely lost (binding percentage<10%) when W30, L49, W50, E56 were mutated to A, indicating these 4 amino acids are critical for its binding to human CLDN18.2. Especially E56 is the most important one to constitute the binding epitope. Besides these 4 critical ones, several other amino acids also effect the binding (binding percentage between 10% and 250%) once they are replaced by alanine, such as R51, F60, E62, R80. The binding of 59A9-C to the site-mutated CLDN18.2 was only partially dependent on E56 (binding percentage about 220). The binding percentage of the mutated CLDN18.2 as compared with the wild-type to the antibodies was summarized in Table 6.

TABLE 6
The binding percentage of mutated CLDN18.2
as compared with wild-type CLDN18.2 (%)

| Mutations on CLDN18.2 | 18B10-HaLa | 59A9-C |
|---|---|---|
| Wild-type | 100.00 | 100.00 |
| Q28A | 28.47 | 46.38 |
| Q29A | 82.85 | 54.83 |
| W30A | 0.38 | 3.20 |
| S31A | 92.24 | 76.85 |
| T32A | 91.56 | 85.59 |
| Q33A | 57.11 | 59.78 |
| D34A | 97.47 | 96.29 |
| L35A | 89.80 | 75.44 |
| Y36A | 79.92 | 78.03 |
| N37A | 61.20 | 60.72 |
| N38A | 66.62 | 57.63 |
| V40A | 103.48 | 128.07 |
| T41A | 60.27 | 41.63 |
| V43A | 36.83 | 59.41 |
| F44A | 59.86 | 70.03 |
| N45A | 36.19 | 35.16 |
| Y46A | 51.55 | 14.98 |
| Q47A | 105.11 | 57.45 |
| L49A | 1.20 | 2.32 |
| W50A | 0.86 | 5.05 |
| R51A | 39.39 | 22.12 |
| S52A | 86.58 | 98.97 |
| V54A | 84.22 | 87.24 |
| R55A | 49.94 | 49.69 |
| E56A | 0.27 | 21.92 |
| S57A | 112.14 | 77.16 |
| S58A | 62.08 | 39.38 |
| F60A | 26.32 | 64.43 |
| T61A | 84.08 | 51.64 |
| E62A | 8.42 | 23.13 |
| R64A | 87.70 | 51.00 |
| Y66A | 53.19 | 52.45 |
| F67A | 76.37 | 79.89 |
| T68A | 70.92 | 71.30 |
| L69A | 88.60 | 54.83 |
| L70A | 75.64 | 82.08 |
| L72A | 56.61 | 53.03 |
| M75A | 75.98 | 73.58 |
| L76A | 43.12 | 52.06 |
| Q77A | 80.06 | 63.42 |
| V79A | 43.72 | 39.11 |
| R80A | 27.27 | 13.64 |

### Example 8 Biodistribution of a Next Generation Nuclide Labeled Monoclonal Antibody Targeting CLDN18.2 (Taking ¹²⁴I-18B10 as an Example) in PDX Mice

¹²⁴I-18B10(0.74MBq, 200 μL for each group) were injected into patient-derived xenografts (PDX) models (Confirmed with claudin 18.2 overexpression, provided by Gastrointestinal oncology department in Peking University Cancer Hospital) formed by transplanting tumor tissue from Gastric cancer patients' tumor into severely immunodeficient mice (NSG) mice via tail vein. The biodistribution experiments were divided into 2 groups, including ¹²⁴I-18B10 in PDX mice for test group, and ¹²⁴I-18B 10 and co-added with unlabeled 18B 10 (200 mg/mice) for blocking group. The heart, liver, lung, kidney, spleen, stomach, bone, muscle, large intestine, small intestine, brain, and tumor of mice were isolated at 240 h after injection. After grinding, the uptake signal was detected and analyzed by 7 counter, and the ID %/g of ¹²⁴I-18B 10 probe was obtained.

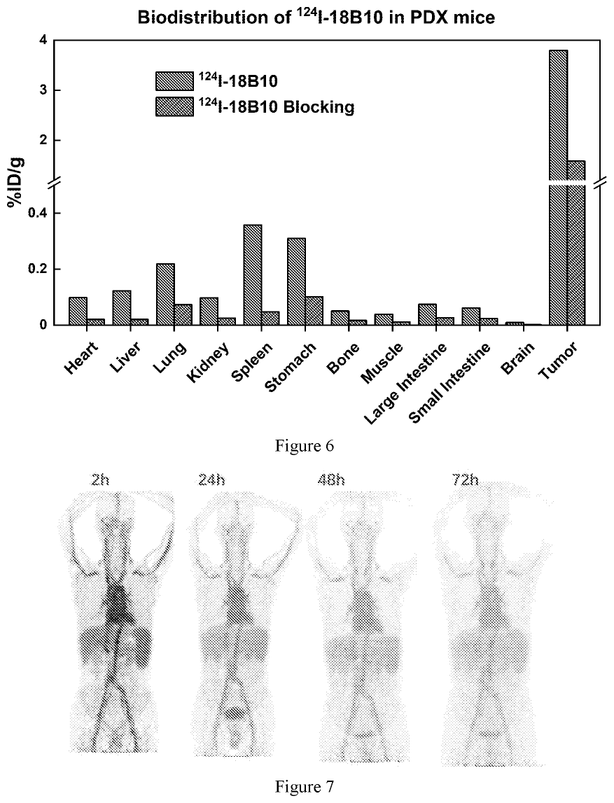

In PDX mice (FIG. 6), ¹²⁴I-18B 10 uptake in tumor was the highest compared with other groups. Due to competition from unlabeled 18B 10, the blocking group showed significantly reduced uptake in both normal tissue and tumor tissue. The result shows ¹²⁴I-18B10 was concentrated on tumor cells, which supports its use in disease diagnosis.

### Example 9 Mapping of a Next Generation Nuclide Labeled Monoclonal Antibody Targeting CLDN18.2 (Taking ¹²⁴I-18B10 as an Example) in Patients

In an investigational clinical study, a patient diagnosed of claudin 18.2-expressing cancer was subject to ¹²⁴I-18B10 PET/CT scan and ¹⁸F-FDG PET/CT within 1 week before or after ¹²⁴I-18B10 PET/CT imaging (11.1-18.5 MBq for each patient, by intravenous injection). For ¹²⁴I-18B10 PET imaging, the thyroid glands of patients were blocked by taking Lugol's potassium iodide (ten drops each time, 3 times a day) 3 days before and 7 days after the administration of ¹²⁴I-18B10. ¹²⁴I-18B10 PET/CT scans were obtained with the Siemens Biograph mCT Flow 64 scanner (Erlangen, Germany) 2 h, 24 h, 72 h, 96 h following administration of ¹²⁴I 18B10. PET images were acquired with scan speeds of 0.8 mm/s, 0.5 mm/s, 0.4 mm/s, 0.3 mm/s and 0.2 mm/s at 2 h, 24 h, 48 h, 72 h respectively, from head to foot or upper thigh, and were reconstructed using the ordered-subset expectation maximization method.

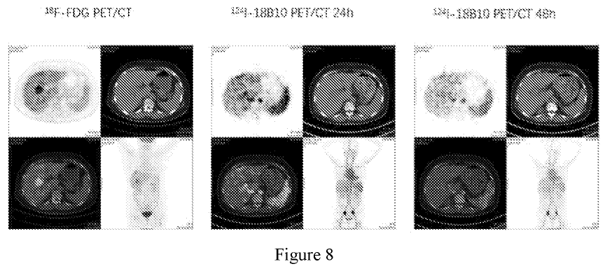

The biodistribution of ¹²⁴I-18B10 in patients were derived from the first five patients' images (FIG. 7). The thyroid glands were sufficiently blocked in the patients. The tracer was concentrated in the blood after injection and decreased over time and the concentration in the spleen and liver was increased over time. The spleen became the organ with the highest activity at 24 h, 48 h and 72 h. there was also tracer uptake in the kidneys and the uptake in the brain, lung and bone was low. No clear tracer uptake was seen in the normal gastric wall. In addition, FIG. 8 showed that ¹²⁴I-18B10 accumulated in the patient's ovarian lesion, which was a claudin 18.2-expressing cancer metastasized to ovary. Compared with ¹⁸F-FDG PET/CT results, results from ¹²⁴I-18B10 PET/CT 24 h and ¹²⁴I-18B10 PET/CT 48 h showed that ¹²⁴I-18B10 had higher uptake in the claudin 18.2-expressing cancer tissue in the ovarian lesion (¹⁸F-FDG PET/CT SUVmax:3(left), 2.9 (right); ¹²⁴I-18B10 PET/CT 24 h SUVmax: 25.1 (left), 22.2 (right); ¹²⁴I-18B10 PET/CT 48 h SUVmax: 12.5 (left), 17.6 (right)).

1. An anti-CLDN18.2 antibody conjugate, comprising anti-CLDN18.2 antibody or an antigen-binding fragment thereof conjugated to a radionuclide, wherein the radionuclide comprises a therapeutic radionuclide or a diagnostic radionuclide.

2. The anti-CLDN18.2 antibody conjugate of claim 1, wherein the therapeutic radionuclide is selected from the group consisting of ¹¹¹In, ^{111m}In, ¹⁷⁷Lu, ²¹²Bi, ²¹³Bi, ²¹¹At, ⁶²Cu, ⁶⁴Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, ¹⁹⁹Au, and ²¹¹Pb, and/or wherein the diagnostic radionuclide is selected from the group consisting of ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹ln, ¹²³I, ¹²⁴I, ¹²⁵I, ¹³¹I, ¹⁴²Pr, ¹⁴³Pr, ¹⁴⁹Pm, ¹⁵³Sm, ^{154″1581}Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴lr, ¹⁹⁸Au, ¹⁹⁹Au, ²¹¹At, ²¹¹Pb, ²¹²Bi, ²¹²Pb, ²¹³Bi, ²²³Ra and ²²⁵Ac.

3. (canceled)

4. The anti-CLDN18.2 antibody conjugate of claim 1, wherein the diagnostic radionuclide is detectable by positron emission tomography (PET) or single-photon emission computerized tomography (SPECT).

5. The anti-CLDN18.2 antibody conjugate of claim 1, wherein the radionuclide is selected from the group consisting of ⁶⁴Cu, ⁶⁷Cu, ⁸⁹Zr, ¹²⁴I, ⁸⁶Y, ⁹⁰Y, ¹¹¹In, ^{123/131}I, ¹⁷⁷Lu, ¹¹C, ¹⁴C, ⁴¹Ca, ⁶⁷Ga, ⁶⁸Ga, ¹³N, ¹⁵O, ⁴⁴Sc, ¹⁸F, ^{99m}Tc.

6-7. (canceled)

8. The anti-CLDN18.2 antibody conjugate of claim 6, wherein the radionuclide is labeled to the antibody or an antigen-binding fragment thereof via a chelator.

9. The anti-CLDN18.2 antibody conjugate of claim 7, wherein the chelator comprises three or more atoms for chelation, wherein each atom is selected from the group consisting of nitrogen, sulfur, oxygen, and phosphorus.

10. The anti-CLDN18.2 antibody conjugate of claim 9, wherein the chelator comprises DFO (derferoxamine), DOTA (1,4,7,10-tetraazacyclododecane-1,4,7,10-tetracetic acid), DTPA (NR-diethylenetriaminepentacetic acid), NOTA (1,4,7-triazacyclononane-1,4,7-acetic acid), 1,4,7,10-tetraazacyclotridecane-N,N′,N″,N′″-tetraacetic acid (herein abbreviated as TRITA); 1,4,8,11-tetraazacyclotetradecane-N,N′,N″,N′″-tetraacetic acid (herein abbreviated as TETA); and 1,5,9,13-tetraazacyclohexadecane-N,N′,N″,N′″-tetraacetic acid (abbreviated herein abbreviated as HETA), ethylenediaminetetraacetic acid (herein abbreviated as EDTA), or diethylenetriaminepentaacetic acid (DTPA).

11. The antibody conjugate of claim 10, wherein the radionuclide is 64Cu or 67Cu and the chelator comprises TETA, NOTA, NODA, or NODGA, or wherein the radionuclide is ⁸⁹Zr and the chelator comprises DFO, or wherein the radionuclide is ¹²⁴I and the chelator comprises EDTA, or wherein the radionuclide is ¹⁷⁷Lu and the chelator comprises DOTA.

12. The antibody conjugate of claim 1, wherein the anti-CLDN18.2 antibody or an antigen-binding fragment thereof comprises heavy chain HCDR1, HCDR2 and HCDR3 and/or light chain LCDR1, LCDR2 and LCDR3 sequences, wherein:
the HCDR1 sequence comprises GYNMN (SEQ ID NO: 1), or TYFIGVG (SEQ ID NO: 13), or a homologue sequence of at least 80% sequence identity thereof,
the HCDR2 sequence comprises X₁IDPYYX₂X₃TX₄YNQKFX₅G (SEQ ID NO: 32), or HIWWNDNKYYNTALKS (SEQ ID NO: 15), or a homologue sequence of at least 80% sequence identity thereof;
the HCDR3 sequence comprises X₆X₇X₈GNAFDY (SEQ ID NO: 33), or MGSGAWFTY (SEQ ID NO: 17), or a homologue sequence of at least 80% sequence identity thereof;
the LCDR1 sequence comprises KSSQX₉LX₁₀NX₁₁GNX₁₂KNYLT (SEQ ID NO: 34) or a homologue sequence of at least 80% sequence identity thereof, the LCDR2 sequence comprises WASTRX₁₃S (SEQ ID NO: 35) or a homologue sequence of at least 80% sequence identity thereof;
the LCDR3 sequence comprises QNDYX₁₄X₁₅PX₁₆T (SEQ ID NO: 36) or a homologue sequence of at least 80% sequence identity thereof;
wherein X₁ is N or Y or H, X₂ is G or V, X₃ is A or G or T, X₄ is R or T or S, X₅ is K or R, X₆ is S or M, X₇ is Y or F, X₈ is Y or H, X₉ is S or N, X₁₀ is L or F, X₁₁ is S or N, X₁₂ is Q or L, X₁₃ is E or K, X₁₄ is S or Y, X₁₅ is F or Y and X₁₆ is F or L.

13. The antibody conjugate of claim 1, wherein the anti-CLDN18.2 antibody or an antigen-binding fragment thereof comprises:
a heavy chain variable region comprises a HCDR1 comprising the sequence of SEQ ID NO: 1, a HCDR2 comprising the sequence of SEQ ID NO: 3, and a HCDR3 comprising the sequence of SEQ ID NO: 5; and a light chain variable region comprises a LCDR1 comprising the sequence of SEQ ID NO: 2, a LCDR2 comprising the sequence of SEQ ID NO: 4, and a LCDR3 comprising the sequence of SEQ ID NO: 6;
a heavy chain variable region comprises a HCDR1 comprising the sequence of SEQ ID NO: 1, a HCDR2 comprising the sequence of SEQ ID NO: 7, and a HCDR3 comprising the sequence of SEQ ID NO: 5; and a light chain variable region comprises a LCDR1 comprising the sequence of SEQ ID NO: 2, a LCDR2 comprising the sequence of SEQ ID NO: 4, and a LCDR3 comprising the sequence of SEQ ID NO: 8;
a heavy chain variable region comprises a HCDR1 comprising the sequence of SEQ ID NO: 1, a HCDR2 comprising the sequence of SEQ ID NO: 9, and a HCDR3 comprising the sequence of SEQ ID NO: 11; and a light chain variable region comprises a LCDR1 comprising the sequence of SEQ ID NO: 10, a LCDR2 comprising the sequence of SEQ ID NO: 4, and a LCDR3 comprising the sequence of SEQ ID NO: 6;
a heavy chain variable region comprises a HCDR1 comprising the sequence of SEQ ID NO: 13, a HCDR2 comprising the sequence of SEQ ID NO: 15, and a HCDR3 comprising the sequence of SEQ ID NO: 17; and a light chain variable region comprises a LCDR1 comprising the sequence of SEQ ID NO: 2, a LCDR2 comprising the sequence of SEQ ID NO: 4, and a LCDR3 comprising the sequence of SEQ ID NO: 12;
a heavy chain variable region comprises a HCDR1 comprising the sequence of SEQ ID NO: 1, a HCDR2 comprising the sequence of SEQ ID NO: 19, and a HCDR3 comprising the sequence of SEQ ID NO: 21; and a light chain variable region comprises a LCDR1 comprising the sequence of SEQ ID NO: 14, a LCDR2 comprising the sequence of SEQ ID NO: 16, and a LCDR3 comprising the sequence of SEQ ID NO: 18; or
a heavy chain variable region comprises a HCDR1 comprising the sequence of SEQ ID NO: 1, a HCDR2 comprising the sequence of SEQ ID NO: 22, and a HCDR3 comprising the sequence of SEQ ID NO: 5; and a light chain variable region comprises a LCDR1 comprising the sequence of SEQ ID NO: 20, a LCDR2 comprising the sequence of SEQ ID NO: 4, and a LCDR3 comprising the sequence of SEQ ID NO: 6.

14-17. (canceled)

18. The antibody conjugate of claim 1, wherein:
the heavy chain variable region comprising the sequence of SEQ ID NO: 23 and a light chain variable region comprising the sequence of SEQ ID NO: 24;
the heavy chain variable region comprises a sequence of SEQ ID NO: 25 and the light chain variable region comprises a sequence of SEQ ID NO: 26;
the heavy chain variable region comprises a sequence of SEQ ID NO: 27 and the light chain variable region comprises a sequence of SEQ ID NO: 28;
the heavy chain variable region comprises a sequence of SEQ ID NO: 29 and the light chain variable region comprises a sequence of SEQ ID NO: 26, or 28;
the heavy chain variable region comprises a sequence of SEQ ID NO: 37 and the light chain variable region comprises a sequence of SEQ ID NO: 38;
the heavy chain variable region comprises a sequence of SEQ ID NO: 39 and the light chain variable region comprises a sequence of SEQ ID NO: 40;
the heavy chain variable region comprises a sequence of SEQ ID NO: 41 and the light chain variable region comprises a sequence of SEQ ID NO: 42;
the heavy chain variable region comprises a sequence of SEQ ID NO: 43 and the light chain variable region comprises a sequence of SEQ ID NO: 44;
the heavy chain variable region comprises a sequence of SEQ ID NO: 45 and the light chain variable region comprises a sequence of SEQ ID NO: 46; or
the heavy chain variable region comprises a sequence of SEQ ID NO: 47 and the light chain variable region comprises a sequence of SEQ ID NO: 48.

19-20. (canceled)

21. The antibody conjugate of claim 12, wherein the anti-CLDN18.2 antibody or an antigen-binding fragment thereof further comprises an immunoglobulin constant region, optionally a constant region of human Ig, or optionally a constant region of human IgG.

22-23. (canceled)

24. The antibody conjugate of claim 1, wherein the anti-CLDN18.2 antibody or an antigen-binding fragment thereof is humanized.

25. The antibody conjugate of claim 1, wherein the anti-CLDN18.2 antibody or an antigen-binding fragment thereof is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv-Fc antibody, an scFv dimer (bivalent diabody), a multispecific antibody, a camelized single domain antibody, a nanobody, a domain antibody, and a bivalent domain antibody.

26. A pharmaceutical composition comprising the antibody conjugate of claim 1, and one or more pharmaceutically acceptable carriers.

27. (canceled)

28. A method of obtaining an image of a site of interest in a subject, the method comprising the steps of:
a) administering to the subject an effective amount of the antibody conjugate of claim 1; and
b) subjecting the site of interest of the subject to positron emission tomography (PET) or SPECT;
c) identifying a detectable signal from the radionuclide in the subject;
d) generating an image of the detectable signal, thereby obtaining an image of the site of interest in the subject.

29-30. (canceled)

31. A method of detecting or visualizing claudin 18.2 expression in a subject in a non-invasive manner, comprising the steps of:
a) administering to the subject an effective amount of the antibody conjugate of claim 1; and
b) subjecting the subject to positron emission tomography (PET) or SPECT;
c) identifying a detectable signal from the radionuclide in a site of interest of the subject;
d) determining or visualizing claudin18.2 expression in the site of interest of the subject based on the identified detectable signal.

32-35. (canceled)

36. A method of monitoring therapeutic efficacy, responsiveness to treatment, or development of resistance or recurrence, or metastasis, in a subject in a non-invasive manner, wherein the subject has received treatment for a therapeutic period, comprising the steps of:
a) administering to the subject an effective amount of the antibody conjugate of claim 1; and
b) subjecting the subject to positron emission tomography (PET) or SPECT;
c) identifying a detectable signal from the radionuclide in a site of interest of the subject;
d) determining post-treatment claudin18.2 expression in a site of interest of the subject based on the identified detectable signal;
e) comparing the post-treatment claudin18.2 expression level or distribution, with a baseline claudin18.2 expression level or distribution, respectively, obtained from the subject before the therapeutic period, to determine post-treatment change in the claudin18.2 expression level or distribution in the subject; and
f) determining the therapeutic efficacy, responsiveness to treatment, or development of resistance or recurrence, or metastasis based on the change determined in step (e).

37-38. (canceled)

39. A method of treating or diagnosing a CLDN18.2 related disease or condition in a subject in need thereof, comprising administering to the subject a therapeutically effective amount of the anti-CLDN18.2 antibody conjugate of claim 1.

40-42. (canceled)

43. A kit comprising a first anti-CLDN18.2 antibody conjugate of claim 1, and a second anti-CLDN18.2 antibody conjugate of claim 1, wherein the first anti-CLDN18.2 antibody conjugate comprises a diagnostic radionuclide, and the second anti-CLDN18.2 antibody conjugate comprises a therapeutic radionuclide.

44-46. (canceled)

47. A method of preparing the anti-CLDN18.2 antibody conjugate of claim 6, comprising reacting an anti-CLDN18.2 antibody or an antigen-binding fragment thereof with an iodide compound labeled with ¹²⁴I, ¹²³I or ¹³¹I, in the presence of an enzymatic or chemical oxidant.

48. (canceled)

49. A method of preparing the anti-CLDN18.2 antibody conjugate of claim 7, comprising conjugating an anti-CLDN18.2 antibody or an antigen-binding fragment thereof with a chelator to obtain a chelator-antibody conjugate, and reacting the chelator-antibody conjugate with ⁶⁴Cu, ¹⁷⁷Lu, or ⁸⁹Zr.

50. (canceled)


SEQUENCE LISTING (73 sequences)

SEQ ID NO: 1 - protein, 5 aa, Mus musculus
GYNMN

SEQ ID NO: 2 - protein, 17 aa, Mus musculus
KSSQSLLNSGNQKNYLT

SEQ ID NO: 3 - protein, 17 aa, Mus musculus
NIDPYYGATRYNQKFKG

SEQ ID NO: 4 - protein, 7 aa, Mus musculus
WASTRES

SEQ ID NO: 5 - protein, 9 aa, Mus musculus
SYYGNAFDY

SEQ ID NO: 6 - protein, 9 aa, Mus musculus
QNDYSFPFT

SEQ ID NO: 7 - protein, 17 aa, Mus musculus
YIDPYYGGTRYNQKFKG

SEQ ID NO: 8 - protein, 9 aa, Mus musculus
QNDYSYPFT

SEQ ID NO: 9 - protein, 17 aa, Mus musculus
HIDPYYVTTTYNQKFRG

SEQ ID NO: 10 - protein, 17 aa, Mus musculus
KSSQSLFNSGNQKNYLT

SEQ ID NO: 11 - protein, 9 aa, Mus musculus
SFYGNAFDY

SEQ ID NO: 12 - protein, 9 aa, Mus musculus
QNDYYYPLT

SEQ ID NO: 13 - protein, 7 aa, Mus musculus
TYFIGVG

SEQ ID NO: 14 - protein, 17 aa, Mus musculus
KSSQSLLNSGNLKNYLT

SEQ ID NO: 15 - protein, 16 aa, Mus musculus
HIWWNDNKYYNTALKS

SEQ ID NO: 16 - protein, 7 aa, Mus musculus
WASTRKS

SEQ ID NO: 17 - protein, 9 aa, Mus musculus
MGSGAWFTY

SEQ ID NO: 18 - protein, 9 aa, Mus musculus
QNDYSYPLT

SEQ ID NO: 19 - protein, 17 aa, Mus musculus
NIDPYYGGTSYNQKFKG

SEQ ID NO: 20 - protein, 17 aa, Mus musculus
KSSQNLLNNGNQKNYLT

SEQ ID NO: 21 - protein, 9 aa, Mus musculus
MYHGNAFDY

SEQ ID NO: 22 - protein, 17 aa, Mus musculus
NIDPYYGGTRYNQKFKG

SEQ ID NO: 23 - protein, 98 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 24 - protein, 95 aa, Homo sapiens
DIVMTQSPDSLAVSLGERATINCKSSQNNKNYLAWYQQKPGQPPKLLIYWASTRESGVPDRFSGSGSGTDFTLTISSLQAEDVAVYYCQQYYSTP

SEQ ID NO: 25 - protein, 118 aa, Homo sapiens
QVQLVQSGAEVKKPGASVKVSCKASGYTFTGYNMNWVRQAPGQGLEWMGNIDPYYGGTSYNQKFKGRVTMTIDKSTSTVYMELSSLRSEDTAVYYCARMYHGNAFDYWGQGTTVTVSS

SEQ ID NO: 26 - protein, 113 aa, Homo sapiens
DIVMTQSPDSLAVSLGERATINCKSSQSLLNSGNLKNYLTWYQQKPGQPPKLLIYWASTRKSGVPDRFSGSGSGTDFTLTISSLQAEDVAVYYCQNDYSYPLTFGGGTKVEIK

SEQ ID NO: 27 - protein, 118 aa, Homo sapiens
QVQLVQSGAEVKKPGASVKVSCKASGYSFTGYNMNWVRQAPGQGLEWMGNIDPYYGGTSYNQKFKGRVTLTIDKSTSTVYMELSSLRSEDTAVYYCARMYHGNAFDYWGQGTTVTVSS

SEQ ID NO: 28 - protein, 113 aa, Homo sapiens
DIVMTQSPDSLAVSLGERATMNCKSSQSLLNSGNLKNYLTWYQQKPGQPPKLLIYWASTRKSGVPDRFTGSGSGTDFTLTISSLQAEDVAVYYCQNDYSYPLTFGGGTKVEIK

SEQ ID NO: 29 - protein, 118 aa, Homo sapiens
QVQLVQSGAEVKKPGASVKVSCKASGYSFTGYNMNWVKQAPGQGLEWIGNIDPYYGGTSYNQKFKGRVTLTIDKSTSTVYMELSSLRSEDTAVYYCARMYHGNAFDYWGQGTTVTVSS

SEQ ID NO: 30 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 31 - protein, 261 aa, Homo sapiens
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 32 - protein, 17 aa, Artificial Sequence
  synthesic
  misc_feature (1)..(1): Xaa can be Asn, Tyr or His
  misc_feature (7)..(7): Xaa can be Gly or Val.
  misc_feature (8)..(8): Xaa can be Ala, Gly or Thr.
  misc_feature (10)..(10): Xaa can be Arg, Thr or Ser.
  misc_feature (16)..(16): Xaa can be Lys or Arg.
XIDPYYXXTXYNQKFXG

SEQ ID NO: 33 - protein, 9 aa, Artificial Sequence
  synthesic
  misc_feature (1)..(1): Xaa can be Ser or Met.
  misc_feature (2)..(2): Xaa can be Tyr or Phe.
  misc_feature (3)..(3): Xaa can be Tyr or His.
XXXGNAFDY

SEQ ID NO: 34 - protein, 17 aa, Artificial Sequence
  synthesic
  misc_feature (5)..(5): Xaa can be Ser or Asn.
  misc_feature (7)..(7): Xaa can be Leu or Phe.
  misc_feature (9)..(9): Xaa can be Ser or Asn.
  misc_feature (12)..(12): Xaa can be Gln or Leu.
KSSQXLXNXGNXKNYLT

SEQ ID NO: 35 - protein, 7 aa, Artificial Sequence
  synthesic
  misc_feature (6)..(6): Xaa can be Glu or Lys.
WASTRXS

SEQ ID NO: 36 - protein, 9 aa, Artificial Sequence
  synthesic
  misc_faeture (5)..(5): Xaa can be Ser or Tyr.
  misc_faeture (6)..(6): Xaa can be Phe or Tyr.
  misc_faeture (8)..(8): Xaa can be Phe or Leu.
QNDYXXPXT

SEQ ID NO: 37 - protein, 118 aa, Mus musculus
EFQLQQSGPELEKPGASVRISCKTSGYSFTGYNMNWVKQSNGESLEWIGNIDPYYGATRYNQKFKGKATLTVDKSSSTAYMQLKSLTSEDSAVYYCARSYYGNAFDYWGQGTTLTVSS

SEQ ID NO: 38 - protein, 113 aa, Mus musculus
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSFPFTFGSGTKLEIK

SEQ ID NO: 39 - protein, 118 aa, Mus musculus
EFQLQQSGPELEKPGASVRISCKTSGYSFTGYNMNWVKQSNGESLEWIGYIDPYYGGTRYNQKFKGKATLTVDKSSSTAYMQLKSLTSEDSAVYYCARSYYGNAFDYWGQGTTLTVSS

SEQ ID NO: 40 - protein, 113 aa, Mus musculus
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 41 - protein, 118 aa, Mus musculus
EFQLQQSGPELEKPGASVKISCKTSGYSFTGYNMNWVKQSNGQSLEWIGHIDPYYVTTTYNQKFRGKATLTVDKSSSTAYMQLKSLTSEDSAVYYCARSFYGNAFDYWGQGTTLTVSS

SEQ ID NO: 42 - protein, 113 aa, Mus musculus
DIVMTQSPSSLTVTAGEKVTMSCKSSQSLFNSGNQKNYLTWYQQKPGQPPKLLIYWASTRESGVPDRFTGSGSGTDFTLTISSVQAEDLAVYYCQNDYSFPFTFGSGTKLEIK

SEQ ID NO: 43 - protein, 119 aa, Mus musculus
QITQKESGPGILQPSQTLSLTCSLSGFSLSTYFIGVGWIRQPSGKGLEWLAHIWWNDNKYYNTALKSRLTISKDTSNNQVFLKIASVDTADTATYYCARMGSGAWFTYWGQGTLVTVSA

SEQ ID NO: 44 - protein, 113 aa, Mus musculus
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYYYPLTFGSGTKLEIK

SEQ ID NO: 45 - protein, 118 aa, Mus musculus
EFQLQQSGPELEKPGASVRISCKTSGYSFTGYNMNWVKQSNGESLEWIGNIDPYYGGTSYNQKFKGKATLTVDKSSSTAYMQLKSLTSEDSAVYYCARMYHGNAFDYWGQGTTLTVSS

SEQ ID NO: 46 - protein, 113 aa, Mus musculus
DIVMTQSPSSLTVTAGEKVTMSCKSSQSLLNSGNLKNYLTWYQQKPGQPPKLLIYWASTRKSGVPDRFTGSGSGTDFTLTLSSVQAEDLAVYYCQNDYSYPLTFGAGTKLELK

SEQ ID NO: 47 - protein, 118 aa, Mus musculus
EFQLQQSGPELEKPGASVRISCKTSGYSFTGYNMNWVKQSNGESLEWIGNIDPYYGGTRYNQKFKGKATLTVDKSSSTAYMQLKSLTSEDSAVYYCARSYYGNAFDYWGQGTTLTVSS

SEQ ID NO: 48 - protein, 113 aa, Mus musculus
DIVMTQSPSSLTVTAGEKVTMSCKSSQNLLNNGNQKNYLTWYQQKPGQPPKLLIYWASTRESGVPDRFTGSGSGTDFILTISSVQAEDLAVYYCQNDYSFPFTFGAGTKLELK

SEQ ID NO: 49 - protein, 330 aa, Homo sapiens [sequence omitted]

SEQ ID NO: 50 - protein, 107 aa, Homo sapiens [sequence omitted]

SEQ ID NO: 51 - protein, 330 aa, Artificial Sequence
  synthesic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 52 - protein, 486 aa, Homo sapiens
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYVMASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 53 - protein, 486 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYVMASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 54 - protein, 30 aa, Artificial Sequence
  synthesic
  misc_feature (28)..(28): Xaa can be Thr or Ser.
QVQLVQSGAEVKKPGASVKVSCKASGYXFT

SEQ ID NO: 55 - protein, 14 aa, Artificial Sequence
  synthesic
  misc_faeture (3)..(3): Xaa can be Arg or Lys.
  misc_faeture (13)..(13): Xaa can be Met or IIe.
WVXQAPGQGLEWXG

SEQ ID NO: 56 - protein, 32 aa, Artificial Sequence
  synthesic
  misc_feature (4)..(4): Xaa can be Met or Leu.
RVTXTIDKSTSTVYMELSSLRSEDTAVYYCAR

SEQ ID NO: 57 - protein, 11 aa, Homo sapiens
WGQGTTVTVSS

SEQ ID NO: 58 - protein, 23 aa, Artificial Sequence
  synthesic
  misc_feature (21)..(21): Xaa can be Ile or Met.
DIVMTQSPDSLAVSLGERATXNC

SEQ ID NO: 59 - protein, 15 aa, Homo sapiens
WYQQKPGQPPKLLIY

SEQ ID NO: 60 - protein, 32 aa, Artificial Sequence
  synthesic
  misc_feature (7)..(7): Xaa can be Ser or Thr.
GVPDRFXGSGSGTDFTLTISSLQAEDVAVYYC

SEQ ID NO: 61 - protein, 10 aa, Homo sapiens
FGGGTKVEIK

SEQ ID NO: 62 - protein, 30 aa, Homo sapiens
QVQLVQSGAEVKKPGASVKVSCKASGYTFT

SEQ ID NO: 63 - protein, 30 aa, Homo sapiens
QVQLVQSGAEVKKPGASVKVSCKASGYSFT

SEQ ID NO: 64 - protein, 14 aa, Homo sapiens
WVRQAPGQGLEWMG

SEQ ID NO: 65 - protein, 14 aa, Homo sapiens
WVKQAPGQGLEWIG

SEQ ID NO: 66 - protein, 32 aa, Homo sapiens
RVTMTIDKSTSTVYMELSSLRSEDTAVYYCAR

SEQ ID NO: 67 - protein, 32 aa, Homo sapiens
RVTLTIDKSTSTVYMELSSLRSEDTAVYYCAR

SEQ ID NO: 68 - protein, 23 aa, Homo sapiens
DIVMTQSPDSLAVSLGERATINC

SEQ ID NO: 69 - protein, 23 aa, Homo sapiens
DIVMTQSPDSLAVSLGERATMNC

SEQ ID NO: 70 - protein, 32 aa, Homo sapiens
GVPDRFSGSGSGTDFTLTISSLQAEDVAVYYC

SEQ ID NO: 71 - protein, 32 aa, Homo sapiens; identical to SEQ ID NO: 70

SEQ ID NO: 72 - protein, 118 aa, Artificial Sequence
  synthesic
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 73 - protein, 113 aa, Artificial Sequence; identical to SEQ ID NO: 40
  synthesic